(19) United States
(12) Patent Application Publication
(10) Pub. No.: US 2008/0213266 A1
(43) Pub. Date: Sep. 4, 2008

(54) Method for Treating CCR4-Related Diseases

(75) Inventors: Shigeru Iida, Tokyo (JP); Mitsuo Satoh, Tokyo (JP); Miho Urakubo, Tokyo (JP); Masako Wakitani, Tokyo (JP); Kazuhisa Uchida, Tokyo (JP); Rinpei Niwa, Tokyo (JP); Kenya Shitara, Tokyo (JP)

Correspondence Address: NIXON & VANDERHYE, PC, 901 NORTH GLEBE ROAD, 11TH FLOOR, ARLINGTON, VA, 22203, US

(73) Assignee: KYOWA HAKKO KOGYO CO., LTD.

(21) Appl. No.: 11/969,555

(22) Filed: Jan. 4, 2008

Related U.S. Application Data
(62) Division of application No. 10/959,310, filed on Oct. 7, 2004, now abandoned.
(60) Provisional application No. 60/572,784, filed on May 21, 2004.

(30) Foreign Application Priority Data
Oct. 8, 2003 (JP) 2003-350162

(51) Int. Cl. A61K 39/395; A61P 43/00

(52) U.S. Cl. 424/133.1

(57) ABSTRACT

The present invention provides an antibody composition comprising an antibody molecule which specifically binds to human CC chemokine receptor 4 (CCR4) and has complex type N-glycoside-linked sugar chains in the Fc region, wherein the complex type N-glycoside-linked sugar chains have a structure in which fucose is not bound to N-acetylglucosamine in the reducing end in the sugar chains; a transformant which produces the antibody composition; a process for producing the antibody composition; and a pharmaceutical composition comprising the antibody composition.

This is a Divisional of application Ser. No. 10/959,310 filed Oct. 7, 2004, which claims priority of provisional application No. 60/572,784 filed May 2, 2004 and claims priority to Japanese patent application No. 2003-350,162 filed with the Japanese Patent Office on Oct. 8, 2003.

### BACKGROUND OF THE INVENTION

1. Field of the Invention

The present invention relates to an antibody composition comprising a recombinant antibody molecule which specifically binds to human CC chemokine receptor 4 (hereinafter referred to as CCR4) and has complex type N-glycoside-linked sugar chains in the Fc region, wherein the complex type N-glycoside-linked sugar chains have a structure in which fucose is not bound to N-acetylglucosamine in the reducing end in the sugar chains; a transformant which produces the antibody composition; a process for producing the antibody composition; and a pharmaceutical composition comprising the antibody composition.

2. Brief Description of the Background Art

Eosinophils, mast cells, and various factors such as IgE, cytokines and chemokines are involved in the pathology of allergic diseases such as bronchial asthma (Am. J. Respir. Crit. Care Med., 152, 2059 (1995), Immunol. Today, 15, 19 (1994)).

Major cytokine-producing cells are CD4-positive helper T cells (hereinafter referred to as Th cells). The activated Th cells are increased in the airway cells and peripheral blood of patients with bronchial asthma (Am. Rev. Respir. Dis., 145, S22 (1992)).

In patients with atopic dermatitis or asthma or animal models of allergic inflammation, IL-4 and IL-5 are increased (Clin. Immunol. Immunopathol, 75, 75 (1995)). These cytokines are produced by Th2 cells among helper T cells. Accordingly, Th2 cells are involved in allergic diseases.

Methods so far developed for the treatment of immunodiseases mediated by Th2 cells include 1) antagonists of cytokines and chemokines, 2) cytokine/chemokine production inhibitors, 3) regulators of inflammatory cell function, and 4) chemical mediator antagonists. However, they inhibit only a part of the complicated network among cytokines, chemokines and inflammatory cells and are not radical treatments.

Thus, in order to inhibit the whole of the complicated network among cytokines, chemokines and inflammatory cells, it is necessary to control the upstream part of allergic reactions, that is, Th2 cells.

Anti-CD4 antibodies are known to control T cells. However, since CD4 is widely expressed in immunocytes, they lack in specificity and have the defect of accompanying potent immunosuppressive effects (Int. Arch. Aller. Immunol., 118, 133 (1999)). Currently, steroid administration is mainly employed for the treatment of Th2-mediated immunodiseases, but steroids have strong side effects.

Human CC chemokine receptor 4 (hereinafter referred to as CCR4) is a seven membrane-spanning G protein-coupled chemokine receptor cloned as K5-5 from human immature basophilic cell line KU-812 (WO96/23068, J. Biol. Chem., 270, 19495 (1995)). Expression of CCR4 in Th2 cells has been confirmed (J. Exp. Med., 187, 129 (1998), J. Immunol, 161, 5111 (1998)) and it is reported that CCR4 is expressed in effector/memory T cells (Int. Immunol., 11, 81 (1999)) and in memory T cells involved in the systemic immunity (Nature, 400, 776 (1999)). These facts strongly suggest that memory T cells activated upon induction of inflammation migrate to the inflammatory site via CCR4 by the action of ligands such as MDC (macrophage-derived chemokine) and TARC (thymus and activation-regulated chemokine) to accelerate activation of other inflammatory cells. It is also reported that, besides immunodiseases, CCR4 is expressed on the surface of tumor cells in leukemia and lymphoma such as adult T cell leukemia and mycosis fungoides (Blood, 96, 685 (2000)).

From the foregoing, therapeutic agents for removing the cells expressing CCR4 from the body of a patient are expected to be effective as therapeutic agents not only for immunodiseases mediated by Th2 cells but also for inflammatory diseases and leukemia mediated by CCR4. As examples of such therapeutic agents targeting at specific molecules, the following antibodies against CCR4 are known.

As antibodies against CCR4, 1G1 antibody and 2B10 antibody having the activity to neutralize receptors (WO00/42074) and an antibody having antagonist activity (WO02/15666) have been reported, which are antibodies produced by hybridomas. An anti-CCR4 human CDR-grafted antibody belonging to a human IgG1 type which specifically reacts to the extracellular region of human CCR4 but does not react with a blood platelet and which shows cytotoxic activity against human CCR4-expressing cells is known (WO03/18635).

It is generally known that when an antibody derived from a non-human animal is administered to human, it is recognized as a foreign substance, whereby side effects are induced (J. Clin. Oncol., 2, 881 (1984), Blood, 65, 1349 (1985), J. Natl. Cancer Inst., 80, 932 (1988), Proc. Natl. Acad. Sci. U.S.A., 82, 1242 (1985)), disappearance of the antibody from the body is accelerated (Blood, 65, 1349 (1985), J. Nucl Med., 26, 1011 (1985), J. Natl. Cancer Inst., 80, 937 (1988)) and thereby the therapeutic effects of the antibody is reduced (J. Immunol., 135, 1530 (1985), Cancer Res., 46, 6489 (1986)).

In order to solve these problems, attempts have been made to convert an antibody derived from a non-human animal into a humanized antibody, such as a human complementarity determining region (hereinafter referred to as CDR)-grafted antibody utilizing recombinant DNA techniques (Nature, 321, 522 (1986)). Humanized antibodies are reported to have reduced immunogenicity (Proc. Natl. Acad. Sci. U.S.A., 86, 4220 (1989)) and have prolonged therapeutic effects (Cancer Res., 56, 1118 (1996), Immunol., 85, 668 (1995)), as compared with antibodies derived from non-human animals.

Since humanized antibodies are prepared utilizing recombinant DNA techniques, they can be prepared as molecules in various forms. For example, a humanized antibody having high effector function can be prepared (Cancer Res., 56, 1118 (1996)).

In recent years, in the treatment of non Hodgkin's leukemia patients by Rituxan and the treatment of mammary cancer patients by Herceptin, when a therapeutic antibody induces high ADCC activity in effector cells of the patients, higher therapeutic effects can be obtained (Blood, 99, 754 (2002); J. Clin. Oncol., 21, 3940 (2003); Clin. Cancer Res., 10, 5650 (2004)).

Antibodies of the human IgG1 subclass express ADCC activity and CDC activity via interactions between the Fc region thereof and antibody receptors (hereinafter referred to as FcγR) or various complement components. It is suggested that in the binding of an antibody to FcγR, the hinge region of the antibody and a sugar chain bound to the second domain of the C region (hereinafter referred to as Cβ2 domain) are important (Chemical Immunology, 65, 88 (1997)).

It is known that there is diversity regarding the addition of galactose to the non-reducing end in a complex type N-glycoside-linked sugar chain bound to the Fc region of an IgG antibody molecule and the addition of fucose to N-acetylglucosamine at the reducing end (Biochemistry, 36, 130 (1997)). In particular, it is reported that the addition of fucose to the N-acetylglucosamine at the reducing end in the sugar chain causes significant decrease of the ADCC activity of the antibody (WO00/61739, J. Biol. Chem., 278, 3466 (2003)).

In general, most of the antibody compositions utilized as pharmaceutical compositions are prepared by recombinant DNA techniques using animal cells such as Chinese hamster ovary tissue-derived CHO cells as host cells, and the sugar chain structure of the expressed antibody compositions differs depending host cells.

It is possible to increase the ratio of sugar chains having a structure in which fucose is not bound to N-acetylglucosamine in the reducing end in the complex type N-glycoside-linked sugar chains bound to the Fc region of antibody molecules in an antibody composition by decreasing or deleting the activity of α1,6-fucosyltransferase (hereinafter referred to as FUT8), GDP-mannose 4,6-dehydratase (hereinafter referred to as GMD) or GDP-4-keto-6-deoxy-D-mannose-3,5-epimerase (hereinafter referred to as Fx) of antibody-producing cells (WO02/31140).

### SUMMARY OF THE INVENTION

An object of the present invention is to provide an antibody composition comprising a recombinant antibody molecule which specifically binds to human CC chemokine receptor 4 (CCR4) and has complex type N-glycoside-linked sugar chains in the Fc region, wherein the complex type N-glycoside-linked sugar chains have a structure in which fucose is not bound to N-acetylglucosamine in the reducing end in the sugar chains; a transformant which produces the antibody composition; a process for producing the antibody composition; and a pharmaceutical composition comprising the antibody composition. Since the anti-CCR4 antibody composition of the present invention includes no antibody molecule having sugar chains to which fucose is bound, it has enhanced effector function. Therefore, it is effective for treatment to reduce CCR4-expressing Th2 cells from patients. The therapeutic use of the antibody in which the effector function is enhanced is also expected to be effective for weakening side effects of patients because it does not require combination use of chemotherapeutic agents or radioisotope-labeled antibodies. Furthermore, reduction of burden on patients and the like are expected by decreasing the dose of the therapeutic agent to patients.

The present invention relates to the following (1) to (55).

(1) An antibody composition comprising a recombinant antibody molecule which specifically binds to human CC chemokine receptor 4 (CCR4) and has complex type N-glycoside-linked sugar chains in the Fc region, wherein the complex type N-glycoside-linked sugar chains have a structure in which fucose is not bound to N-acetylglucosamine in the reducing end in the sugar chains.
(2) The antibody composition according to (1), wherein the complex type N-glycoside-linked sugar chains are sugar chains in which 1-position of fucose is not bound to 6-position of N-acetylglucosamine in the reducing end through α-bond in the sugar chains.
(3) The antibody composition according to (1), which specifically binds to an extracellular region of human CC chemokine receptor 4 (CCR4).
(4) The antibody composition according to (3), wherein the extracellular region is an extracellular region selected from the group consisting of the sequences at positions 1 to 39, positions 98 to 112, positions 176 to 206 and positions 271 to 284 of the amino acid sequence represented by SEQ ID NO:36.
(5) The antibody composition according to (3) or (4), wherein the extracellular region is an epitope existing at positions 2 to 29 of the amino acid sequence represented by SEQ ID NO:36.
(6) The antibody composition according to any one of (3) to (5), wherein the extracellular region is an epitope existing at positions 13 to 29 of the amino acid sequence represented by SEQ ID NO:36.
(7) The antibody composition according to any one of (3) to (6), wherein the extracellular region is an epitope existing at positions 13 to 25 of the amino acid sequence represented by SEQ ID NO:36.
(8) The antibody composition according to (7), which has lower binding activity to a peptide comprising the sequence at positions 13 to 25 of the amino acid sequence represented by SEQ ID NO:36 wherein at least one of tyrosine residues at positions 16, 19, 20 and 22 is sulfated, in comparison with its binding activity to a peptide comprising the sequence at positions 13 to 25 of the amino acid sequence represented by SEQ ID NO:36.
(9) The antibody composition according to any one of (1) to (8), which has no reactivity to a human blood platelet.
(10) The antibody composition according to any one of (1) to (9), which specifically binds to an extracellular region of human CC chemokine receptor 4 (CCR4) and has no inhibition activity on binding of a CCR4 ligand which is TARC (thymus and activation-regulated chemokine) or MDC (macrophage-derived chemokine) to CCR4.
(11) The antibody composition according to any one of (1) to (10), which specifically binds to a human CC chemokine receptor 4 (CCR4)-expressing cell.
(12) The antibody composition according to any one of (1) to (11), which has cytotoxic activity against a human CC chemokine receptor 4 (CCR4)-expressing cell.
(13) The antibody composition according to any one of (1) to (12), which has higher cytotoxic activity against a human CC chemokine receptor 4 (CCR4)-expressing cell than a monoclonal antibody produced by a non-human animal-derived hybridoma.
(14) The antibody composition according to any one of (11) to (13), wherein the human CC chemokine receptor 4 (CCR4)-expressing cell is a helper T cell.
(15) The antibody composition according to (12) or (13), wherein the cytotoxic activity is antibody-dependent cell-mediated cytotoxic (ADCC) activity.
(16) The antibody composition according to any one of (1) to (15), which comprises complementarity determining region (CDR) 1, CDR 2 and CDR 3 of an antibody molecule heavy chain (H chain) variable region (V region) consisting of the amino acid sequences represented by SEQ ID NOs:14, 15 and 16, respectively.
(17) The antibody composition according to any one of (1) to (16), which comprises complementarity determining region (CDR) 1, CDR 2 and CDR 3 of an antibody molecule light chain (L chain) variable region (V region) consisting of the amino acid sequences represented by SEQ ID NOs:17, 18 and 19, respectively.
(18) The antibody composition according to any one of (1) to (17), which comprises complementarity determining region (CDR) 1, CDR 2 and CDR 3 of an antibody molecule heavy chain (H chain) variable region (V region) consisting of the amino acid sequences represented by SEQ ID NOs:14, 15 and 16, respectively; and CDR 1, CDR 2 and CDR 3 of an antibody molecule light chain (L chain) V region consisting of the amino acid sequences represented by SEQ ID NOs:17, 18 and 19, respectively.
(19) The antibody composition according to any one of (1) to (18), wherein the recombinant antibody is a human chimeric antibody or a human CDR-grafted antibody.
(20) The antibody composition according to (19), wherein the human chimeric antibody comprises CDRs of heavy chain (H chain) variable region (V region) and light chain (L chain) V region of a monoclonal antibody which specifically binds to human CC chemokine receptor 4 (CCR4).
(21) The antibody composition according to (20), wherein the heavy chain (H chain) variable region (V region) of the antibody molecule comprises the amino acid sequence represented by SEQ ID NO:21.
(22) The antibody composition according to (20), wherein the light chain (L chain) variable region (V region) of the antibody molecule comprises the amino acid sequence represented by SEQ ID NO:23.
(23) The antibody composition according to any one of (20) to (22), wherein the heavy chain (H chain) variable region (V region) of the antibody molecule comprises the amino acid sequence represented by SEQ ID NO:21 and the light chain (L chain) V region of the antibody molecule comprises the amino acid sequence represented by SEQ ID NO:23.
(24) The antibody composition according to (19), wherein the human CDR-grafted antibody comprises CDRs of H chain V region and L chain V region of a monoclonal antibody which specifically binds to human CC chemokine receptor 4 (CCR4).
(25) The antibody composition according to (24), wherein the human CDR-grafted antibody comprises CDRs of heavy chain (H chain) variable region (V region) and light chain (L chain) V region of a monoclonal antibody which specifically binds to human CC chemokine receptor 4 (CCR4), and framework regions (FRs) of H chain V region and L chain V region of a human antibody.
(26) The antibody composition according to (24) or (25), which comprises CDRs of heavy chain (H chain) variable region (V region) and light chain (L chain) V region of a monoclonal antibody which specifically binds to human CC chemokine receptor 4 (CCR4), FRs of H chain V region and L chain V region of a human antibody, and H chain constant region (C region) and L chain C region of a human antibody.
(27) The antibody composition according to any one of (24) to (26), wherein the heavy chain (H chain) variable region (V region) of the antibody molecule comprises the amino acid sequence represented by SEQ ID NO:24 or an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24.
(28) The antibody composition according to any one of (24) to (26), wherein the heavy chain (H chain) variable region (V region) of the antibody molecule comprises the amino acid sequence represented by SEQ ID NO:25 or an amino acid sequence in which at least one amino acid residue selected from the group consisting of Thr at position 28 and Ala at position 97 is substituted with another amino acid residue in the amino acid sequence represented by SEQ ID NO:25.
(29) The antibody composition according to any one of (24) to (26), wherein the light chain (L chain) variable region (V region) of the antibody molecule comprises the amino acid sequence represented by SEQ ID NO:26 or an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26.
(30) The antibody composition according to any one of (24) to (27) and (29), wherein the heavy chain (H chain) variable region (V region) of the antibody molecule comprises the amino acid sequence represented by SEQ ID NO:24 or an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24; and the light chain (L chain) V region of the antibody molecule comprises the amino acid sequence represented by SEQ ID NO:26 or an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26.
(31) The antibody composition according to any one of (24) to (26), (28) and (29), wherein the heavy chain (H chain) variable region (V region) of the antibody molecule comprises the amino acid sequence represented by SEQ ID NO:25 or an amino acid sequence in which at least one amino acid residue selected from the group consisting of Thr at position 28 and Ala at position 97 is substituted with another amino acid residue in the amino acid sequence represented by SEQ ID NO:25; and the light chain (L chain) V region of the antibody molecule comprises the amino acid sequence represented by SEQ ID NO:26 or an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26.
(32) The antibody composition according to any one of (24) to (28) and (29) to (31), wherein the heavy chain (H chain) variable region (V region) of the antibody molecule comprises an amino acid sequence selected from the group consisting of the amino acid sequences represented by SEQ ID NOs:24, 25, 27, 28, 29, 30, 31 and 32.
(33) The antibody composition according to any one of (24) to (26) and (29) to (31), wherein the light (L chain) variable region (V region) of the antibody molecule comprises an amino acid sequence selected from the group consisting of the amino acid sequences represented by SEQ ID NOs:26, 33, 34 and 35.
(34) The antibody composition according to any one of (24) to (26), (32) and (33), wherein the heavy chain (H chain) variable region (V region) of the antibody molecule comprises an amino acid sequence selected from the group consisting of the amino acid sequences represented by SEQ ID NOs:24, 25, 27, 28, 29, 30, 31 and 32; and the light chain (L chain) V region of the antibody molecule comprises an amino acid sequence selected from the group consisting of the amino acid sequences represented by SEQ ID NOs:33, 34 and 35.
(35) The antibody composition according to any one of (24) to (26), wherein the heavy chain (H chain) variable region (V region) of the antibody molecule comprises the amino acid sequence represented by SEQ ID NO:27 or 28, and the light chain (L chain) V region of the antibody molecule comprises the amino acid sequence represented by SEQ ID NO:35.
(36) A transformant producing the antibody composition according to any one of (1) to (35), which is obtained by introducing a DNA encoding an antibody molecule which specifically binds to human CC chemokine receptor 4 (CCR4) into a host cell.
(37) The transformant according to (36), wherein the host cell is a cell in which genome is modified so as to have deleted activity of an enzyme relating to the synthesis of an intracellular sugar nucleotide, GDP-fucose, or an enzyme relating to the modification of a sugar chain in which 1-position of fucose is bound to 6-position of N-acetylglucosamine in the reducing end through α-bond in a complex type N-glycoside-linked sugar chain.
(38) The transformant according to (36), wherein the host cell is a cell in which all of alleles on a genome encoding an enzyme relating to the synthesis of an intracellular sugar nucleotide, GDP-fucose, or an enzyme relating to the modification of a sugar chain in which 1-position of fucose is bound to 6-position of N-acetylglucosamine in the reducing end through α-bond in a complex type N-glycoside-linked sugar chain are knocked out.
(39) The transformant according to (37) or (38), wherein the enzyme relating to the synthesis of an intracellular sugar nucleotide, GDP-fucose, is an enzyme selected from the group consisting of GDP-mannose 4,6-dehydratase (GMD) and GDP-4-keto-6-deoxy-D-mannose 3,5-epimerase (Fx).
(40) The transformant according to (39), wherein the GDP-mannose 4,6-dehydratase is a protein encoded by a DNA selected from the group consisting of the following (a) and (b):
(a) a DNA comprising the nucleotide sequence represented by SEQ ID NO:1;
(b) a DNA which hybridizes with the DNA consisting of the nucleotide sequence represented by SEQ ID NO:1 under stringent conditions and which encodes a protein having GDP-mannose 4,6-dehydratase activity.
(41) The transformant according to (39), wherein the GDP-mannose 4,6-dehydratase is a protein selected from the group consisting of the following (a) to (c):
(a) a protein comprising the amino acid sequence represented by SEQ ID NO:2;
(b) a protein consisting of an amino acid sequence wherein one or more amino acid residues are deleted, substituted, inserted and/or added in the amino acid sequence represented by SEQ ID NO:2 and having GDP-mannose 4,6-dehydratase activity;
(c) a protein consisting of an amino acid sequence which has 80% or more homology to the amino acid sequence represented by SEQ ID NO:2 and having GDP-mannose 4,6-dehydratase activity.
(42) The transformant according to (39), wherein the GDP-4-keto-6-deoxy-D-mannose 3,5-epimerase is a protein encoded by a DNA selected from the group consisting of the following (a) and (b):
(a) a DNA comprising the nucleotide sequence represented by SEQ ID NO:3;
(b) a DNA which hybridizes with the DNA consisting of the nucleotide sequence represented by SEQ ID NO:3 under stringent conditions and which encodes a protein having GDP-4-keto-6-deoxy-D-mannose 3,5-epimerase activity.
(43) The transformant according to (39), wherein the GDP-4-keto-6-deoxy-D-mannose 3,5-epimerase is a protein selected from the group consisting of the following (a) to (c):
(a) a protein comprising the amino acid sequence represented by SEQ ID NO:4;
(b) a protein consisting of an amino acid sequence wherein one or more amino acid residues are deleted, substituted, inserted and/or added in the amino acid sequence represented by SEQ ID NO:4 and having GDP-4-keto-6-deoxy-D-mannose 3,5-epimerase activity;
(c) a protein consisting of an amino acid sequence which has 80% or more homology to the amino acid sequence represented by SEQ ID NO:4 and having GDP-4-keto-6-deoxy-D-mannose 3,5-epimerase activity.
(44) The transformant according to (37) or (38), wherein the enzyme relating to the modification of a sugar chain in which 1-position of fucose is bound to 6-position of N-acetylglucosamine in the reducing end through α-bond in a complex type N-glycoside-linked sugar chain is α1,6-fucosyltransferase.
(45) The transformant according to (44), wherein the α1,6-fucosyltransferase is a protein encoded by a DNA selected from the group consisting of the following (a) to (d):
(a) a DNA comprising the nucleotide sequence represented by SEQ ID NO:5;
(b) a DNA comprising the nucleotide sequence represented by SEQ ID NO:6;
(c) a DNA which hybridizes with the DNA consisting of the nucleotide sequence represented by SEQ ID NO:5 under stringent conditions and which encodes a protein having α1,6-fucosyltransferase activity;
(d) a DNA which hybridizes with the DNA consisting of the nucleotide sequence represented by SEQ ID NO:6 under stringent conditions and which encodes a protein having α1,6-fucosyltransferase activity.
(46) The transformant according to (44), wherein the α1,6-fucosyltransferase is a protein selected from the group consisting of the following (a) to (f):
(a) a protein comprising the amino acid sequence represented by SEQ ID NO:7;
(b) a protein comprising the amino acid sequence represented by SEQ ID NO:8;
(c) a protein consisting of an amino acid sequence wherein one or more amino acid residues are deleted, substituted, inserted and/or added in the amino acid sequence represented by SEQ ID NO:7 and having α1,6-fucosyltransferase activity;
(d) a protein consisting of an amino acid sequence wherein one or more amino acid residues are deleted, substituted, inserted and/or added in the amino acid sequence represented by SEQ ID NO:8 and having α1,6-fucosyltransferase activity;
(e) a protein consisting of an amino acid sequence which has 80% or more homology to the amino acid sequence represented by SEQ ID NO:7 and having α1,6-fucosyltransferase activity;
(f) a protein consisting of an amino acid sequence which has 80% or more homology to the amino acid sequence represented by SEQ ID NO:8 and having α1,6-fucosyltransferase activity.
(47) The transformant according to (46), wherein the transformant is FERM BP-8467.
(48) The transformant according to any one of (36) to (47), wherein the host cell is a cell selected from the group consisting of the following (a) to (i):
(a) a CHO cell derived from Chinese hamster ovary tissue;
(b) a rat myeloma cell line YB2/3HL.P2.G11.16Ag.20 cell;
(c) a mouse myeloma cell line NS0 cell;
(d) a mouse myeloma cell line SP2/0-Ag14 cell;
(e) a BHK cell derived from Syrian hamster kidney tissue;
(f) an antibody-producing hybridoma cell;
(g) a human leukemia cell line Namalwa cell;
(h) an embryonic stem cell;
(i) a fertilized egg cell.
(49) A process for producing the antibody composition according to any one of (1) to (35), which comprises culturing the transformant according to any one of (36) to (48) in a medium to form and accumulate the antibody composition in the culture, and recovering and purifying the antibody composition from the culture.
(50) The antibody composition according to any one of (1) to (35), which is obtained by the process according to (49).
(51) A pharmaceutical composition comprising the antibody composition according to any one of (1) to (35) and (50) as an active ingredient.
(52) A therapeutic agent for diseases relating to a human CC chemokine receptor 4 (CCR4), comprising the antibody composition according to any one of (1) to (35) and (50) as an active ingredient.
(53) The therapeutic agent according to (52), wherein the diseases relating to a human CC chemokine receptor 4 (CCR4) are cancer or inflammatory diseases.
(54) A method for treating diseases related to a human CC chemokine receptor 4 (CCR4), which comprises administering to a patient the antibody composition according to any one of (1) to (35) and (50).
(55) Use of the antibody composition according to any one of (1) to (35) and (50) to produce a therapeutic agent for diseases related to a human CC chemokine receptor 4 (CCR4).

### BRIEF DESCRIPTION OF THE DRAWINGS

FIG. 1 shows the steps for constructing plasmid pKOFUT8Neo.

FIG. 2 shows the result of genomic Southern analysis of a hemi-knockout clone wherein one copy of the FUT8 allele was disrupted in CHO/DG44 cell. The lanes respectively show the following, from left to right: molecular weight marker, hemi-knockout clone 50-10-104, and parent cell CHO/DG44.

FIG. 3 shows the result of genomic Southern analysis of double-knockout clone WK704 wherein both FUT8 alleles were disrupted in CHO/DG44 cell. The arrow indicates the detection spot of a positive fragment resulting from homologous recombination.

FIG. 4 shows the result of genomic Southern analysis of a clone obtained by removing a drug-resistance gene from a double-knockout clone wherein both FUT8 alleles were disrupted in CHO/DG44 cell. The lanes respectively show the following, from left to right: molecular weight marker, drug resistance gene-removed double-knockout clone 4-5-C3, double-knockout clone WK704, hemi-knockout clone 50-10-104, and parent cell CHO/DG44.

FIG. 5 shows the schematic view of the produced expression vector for the anti-CCR4 human CDR-grafted antibody.

FIG. 6 shows the reactivity of purified Ms705/CCR4 antibody and DG44/CCR4 antibody at varied concentrations to a CCR4-partial peptide measured by ELISA. The numbers on the abscissa indicate the antibody concentration and those on the ordinate indicate the absorbance at each antibody concentration. □ corresponds to the DG44/CCR4 antibody, and ▪ corresponds to the Ms705/CCR4 antibody.

FIG. 7 shows the ADCC activity of purified Ms705/CCR4 antibody and DG44/CCR4 antibody at varied concentrations to human CCR4-highly expressing cells (CCR4/EL-4). The numbers on the abscissa indicate the antibody concentration and those on the ordinate indicate the cytotoxic activity at each antibody concentration. corresponds to the DG44/CCR4 antibody, and ∘ corresponds to the Ms705/CCR4 antibody.

FIG. 8 shows in vitro ADCC activities of anti-CCR4 human CDR-grafted antibody compositions prepared by adding 0 to 300 ng/ml of the DG44/CCR4 antibody or the Ms705/CCR4 antibody to 3.7 ng/ml of the Ms705/CCR4 antibody, to the CCR4/EL4 cells. The ordinate shows the cytotoxic activity, and the abscissa shows the added antibody concentration. corresponds to the activity of the antibody composition prepared by adding the DG44/CCR4 antibody to 3.7 ng/ml of the Ms705/CCR4 antibody, and ∘ corresponds to the activity of the antibody composition prepared by adding the Ms705/CCR4 antibody to 3.7 ng/ml of the Ms705/CCR4 antibody. In the drawing, * corresponds to an antibody composition in which the ratio of an antibody having a sugar chain in which fucose is not bound is 20% or more, among the antibody compositions prepared by adding the DG44/CCR4 antibody to 3.7 ng/ml of the Ms705/CCR4 antibody.

FIG. 9 shows in vitro ADCC activities of an antibody composition comprising the Ms705/CCR4 antibody alone, or an antibody composition prepared by mixing the Ms705/CCR4 antibody with a 9-fold amount of the DG44/CCR4 antibody, to CCR4/EL4 cells. The ordinate shows the cytotoxic activity. The numerical values plotted as the abscissa show the concentration of the Ms705/CCR4 antibody (ng/ml), the concentration of the added DG44/CCR4 antibody (ng/ml) and the total antibody concentration (ng/ml), respectively, from the upper row. □ corresponds to the activity of the antibody composition comprising the Ms705/CCR4 antibody alone, and ▪ corresponds to the activity of the antibody composition prepared by mixing the Ms705/CCR4 antibody with a 9-fold amount of the DG44/CCR4 antibody.

### DETAILED DESCRIPTION OF THE INVENTION

An example of the antibody composition of the present invention comprising a recombinant antibody molecule which specifically binds to human CCR4 and has complex type N-glycoside-linked sugar chains in the Fc region, wherein the complex type N-glycoside-linked sugar chains have a structure in which fucose is not bound to N-acetylglucosamine in the reducing end in the sugar chains, is an antibody composition wherein the complex type N-glycoside linked sugar chains have a structure in which 1-position of fucose is not bound to 6-position of N-acetylglucosamine in the reducing end through α-bond.

An antibody molecule has the Fc region, to which N-glycoside-linked sugar chains are bound. Therefore, two sugar chains are bound to one antibody molecule.

The N-glycoside-linked sugar chains include complex type sugar chains having one or multiple number of parallel galactose-N-acetylglucosamine (hereinafter referred to as Gal-GlcNAc) side chains in the non-reducing end of the core structure and having sialic acid, bisecting N-acetylglucosamine or the like in the non-reducing end of Gal-GlcNAc.

In the present invention, the complex type N-glycoside-linked sugar chain is represented by the following chemical formula 1.

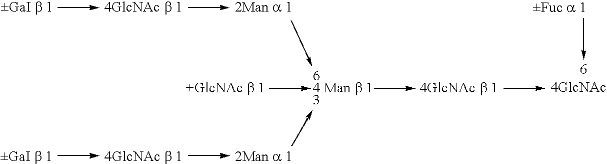

In the present invention, the sugar chain to which fucose is not bound includes a sugar chain represented by the above chemical formula in which fucose is not bound to N-acetylglucosamine in the reducing end. The sugar chain in the non-reducing end may have any structure.

Accordingly, the antibody composition of the present invention comprises an antibody molecule having the same sugar chain structure or antibody molecules having different sugar chain structures, so long as the antibody composition has the above sugar chain structure.

The expression “fucose is not bound to the N-acetylglucosamine in the reducing end in the sugar chains” as used herein means that fucose is not substantially bound thereto. The “antibody composition in which fucose is not substantially bound” specifically refers to an antibody composition in which fucose is not substantially detected, i.e., the content of fucose is below the detection limit, when subjected to the sugar chain analysis described in 4 below. The antibody composition of the present invention in which fucose is not bound to the N-acetylglucosamine in the reducing end in the sugar chains has high ADCC activity.

The ratio of an antibody molecule having sugar chains in which fucose is not bound to the N-acetylglucosamine in the reducing end in an antibody composition comprising an antibody molecule having complex type N-glycoside-linked sugar chains in the Fc region can be determined by releasing the sugar chains from the antibody molecule by known methods such as hydrazinolysis and enzyme digestion [Seibutsukagaku Jikkenho (Biochemical Experimentation Methods) 23—Totanpakushitsu Tosa Kenkyuho (Methods of Studies on Glycoprotein Sugar Chains), Gakkai Shuppan Center, edited by Reiko Takahashi (1989)], labeling the released sugar chains with a fluorescent substance or radioisotope, and separating the labeled sugar chains by chromatography. Alternatively, the released sugar chains may be analyzed by the HPAED-PAD method [J. Liq. Chromatogr., 6, 1577 (1983)] to determine the ratio.

The antibody compositions of the present invention include recombinant antibody compositions comprising a recombinant antibody molecule which specifically binds to an extracellular region of human CCR4, wherein the complex type N-glycoside-linked sugar chains have a structure in which fucose is not bound to N-acetylglucosamine in the reducing end in the sugar chains. Among these, the antibody composition which has no reactivity to a human blood platelet is preferred.

Examples of the extracellular region of human CCR4 include a polypeptide or peptide comprising the amino acid sequence at positions 1 to 39, 98 to 112, 176 to 206 or 271 to 284 of the amino acid sequence represented by SEQ ID NO:36, preferably, comprising the sequence at positions 2 to 29 of the amino acid sequence represented by SEQ ID NO:36 (SEQ ID NO:37), more preferably, comprising the sequence at positions 12 to 29 of the amino acid sequence represented by SEQ ID NO:36 (SEQ ID NO:38), and most preferably, represented by the sequence at positions 12 to 25 of the amino acid sequence represented by SEQ ID NO:36. Accordingly, the antibody composition of the present invention is preferably an antibody composition which specifically binds to a polypeptide or peptide comprising the amino acid sequence at positions 1 to 39, 98 to 112, 176 to 206 or 271 to 284 of the amino acid sequence represented by SEQ ID NO:36, preferably, comprising the sequence at positions 2 to 29 of the amino acid sequence represented by SEQ ID NO:36 (SEQ ID NO:37), more preferably, comprising the sequence at positions 12 to 29 of the amino acid sequence represented by SEQ ID NO:36 (SEQ ID NO:38), and most preferably, comprising the sequence at positions 12 to 25 of the amino acid sequence represented by SEQ ID NO:36, wherein the complex type N-glycoside-linked sugar chains have a structure in which fucose is not bound to N-acetylglucosamine in the reducing end in the sugar chains.

The expression “having no reactivity to a human blood platelet” means that an antibody does not substantially react with a human blood platelet, and specifically, the reactivity is not observed in an analysis using an apparatus capable of detecting the binding of an antibody to a cell such as a flow cytometer.

Also, preferably, the antibody compositions of the present invention include an antibody composition which has lower binding activity to a peptide comprising the sequence at positions 13 to 25 of the amino acid sequence represented by SEQ ID NO:36 wherein at least one of tyrosine residues at positions 16, 19, 20 and 22 is sulfated, in comparison with its binding activity to a peptide comprising the sequence at positions 13 to 25 of the amino acid sequence represented by SEQ ID NO:36.

The antibody compositions of the present invention also include a recombinant antibody composition which have no activity of inhibiting binding of TARC or MDC, which are CCR4 ligands, to CCR4. The expression “not having the activity to inhibit the binding of CCR4 ligands such as TARC and MDC to CCR4” means that the antibody composition is not substantially capable of inhibiting the binding of the ligand to the receptor, and specifically, the antibody composition even at a high concentration does not affect the binding of the ligand to the receptor. Herein, the high concentration means an antibody composition at 50 μg/ml or more.

Furthermore, the antibody compositions of the present invention include antibody compositions comprising a recombinant antibody molecule which specifically binds to a cell in which human CCR4 is expressed (hereinafter referred to as human CCR4-expressing cell) and has complex type N-glycoside-linked sugar chains in the Fc region, wherein the complex type N-glycoside-linked sugar chains have a structure in which fucose is not bound to N-acetylglucosamine in the reducing end in the sugar chains, and the like. Among such antibody compositions, they are preferably antibody compositions having cytotoxic activity against a human CCR4-expressing cell.

The human CCR4-expressing cells include helper T2 cells (hereinafter referred to as Th2 cells) and the like. Th2 cells are cells producing interleukin-4 (hereinafter referred to as IL-4), interleukin-5 (hereinafter referred to as IL-5) and interleukin-13 (hereinafter referred to as IL-13).

The cytotoxic activity includes complement-dependent cytotoxic activity (hereinafter referred to as CDC activity), antibody-dependent cell-mediated cytotoxic activity (hereinafter referred to as ADCC activity), and the like.

The antibody compositions having cytotoxic activity against a human CCR4-expressing cell, wherein the complex type N-glycoside-linked sugar chains have a structure in which fucose is not bound to N-acetylglucosamine in the reducing end in the sugar chains are capable of injuring Th2 cells which are CCR4-expressing cells by the cytotoxic activity possessed by the antibody composition to thereby eliminate the Th2 cells. Accordingly, the antibody composition can suppress production of interleukins such as IL-4, IL-5 and IL-13 from the Th-2 cells.

The antibody compositions of the present invention include compositions of human chimeric antibodies, compositions of human CDR-grafted antibodies, compositions of human antibodies and compositions of fragments of such antibodies.

The “human chimeric antibody” refers to an antibody comprising VH and VL of an antibody derived from a non-human animal, and CH and CL of a human antibody. As the non-human animal, any animal can be used so long as hybridomas can be prepared from the animal. Suitable animals include mouse, rat, hamster, rabbit and the like.

The human chimeric antibody composition of the present invention can be produced by obtaining cDNAs encoding VH and VL of a non-human animal-derived antibody which specifically binds to human CCR4, inserting the cDNAs into an expression vector for animal cells which carries genes encoding CH and CL of a human antibody to construct a human chimeric antibody expression vector, and introducing the vector into an animal cell to induce expression.

As the CH for the human chimeric antibody, any CH of antibodies belonging to human immunoglobulin (hereinafter referred to as hIg) may be used. Preferred are those of antibodies belonging to the hIgG class, which may be of any subclass, e.g., hIgG1, hIgG2, hIgG3 and hIgG4. As the CL for the human chimeric antibody, any CL of antibodies belonging to hIg, such as class κ or class λ, may be used.

Examples of the human chimeric antibody compositions of the present invention which specifically bind to human CCR4 include: an anti-human CCR4 chimeric antibody comprising CDR1, CDR2 and CDR3 of VH consisting of the amino acid sequences represented by SEQ ID NOs:14, 15 and 16, respectively, and/or CDR1, CDR2 and CDR3 of VL consisting of the amino acid sequences represented by SEQ ID NOs:17, 18 and 19, respectively; an anti-human CCR4 chimeric antibody wherein the VH of the antibody comprises the amino acid sequence represented by SEQ ID NO:21 and/or the VL of the antibody comprises the amino acid sequence represented by SEQ ID NO:23; and an anti-human CCR4 chimeric antibody composition wherein the VH of the antibody consists of the amino acid sequence represented by SEQ ID NO:21, the CH of the human antibody consists of an amino acid sequence of the hIgG1 subclass, the VL of the antibody consists of the amino acid sequence represented by SEQ ID NO:23, and the CL of the human antibody consists of an amino acid sequence of the K class.

The “human CDR-grafted antibody” refers to an antibody in which CDRs of VH and VL of an antibody derived from a non-human animal are grafted into appropriate sites in VH and VL of a human antibody.

The human CDR-grafted antibody composition of the present invention can be produced by constructing cDNAs encoding V regions in which CDRs of VH and VL of a non-human animal-derived antibody which specifically binds to CCR4 are grafted into frameworks (hereinafter referred to as FR) of VH and VL of an arbitrary human antibody, inserting the resulting cDNAs into an expression vector for animal cells which has DNAs encoding the heavy chain constant region (hereinafter referred to as CH) and the light chain constant region (hereinafter referred to as CL) of a human antibody to construct a human CDR-grafted antibody expression vector, and introducing the expression vector into an animal cell to induce expression.

As the FR amino acid sequences of VH and VL of a human antibody, any of those derived from human antibodies can be used. Suitable sequences include the FR amino acid sequences of VH and VL of human antibodies registered in databases such as Protein Data Bank, and the amino acid sequences common to all FR subgroups of VH and VL of human antibodies (Sequences of Proteins of Immunological Interest, US Dept. Health and Human Services, 1991).

As the CH for the antibody of the present invention, any CH of antibodies belonging to hIg may be used. Preferred are those of antibodies belonging to the hIgG class, which may be of any subclass, e.g., hIgG1, hIgG2, hIgG3 and hIgG4. As the CL for the human CDR-grafted antibody, any CL of antibodies belonging to hIg, e.g., class K or class A, may be used.

An example of the human CDR-grafted antibody composition of the present invention is a human CDR-grafted antibody or antibody fragment composition comprising CDR1, CDR2 and CDR3 of VH consisting of the amino acid sequences represented by SEQ ID NOs:14, 15 and 16, respectively; and/or CDR1, CDR2 and CDR3 of VL consisting of the amino acid sequences represented by SEQ ID NOs:17, 18 and 19, respectively.

Preferred human CDR-grafted antibody compositions include: a human CDR-grafted antibody composition, wherein the VH of the antibody comprises the amino acid sequence represented by SEQ ID NO:24 or an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24, a human CDR-grafted antibody composition, wherein the VH of the antibody comprises the amino acid sequence represented by SEQ ID NO:25 or an amino acid sequence in which at least one amino acid residue selected from the group consisting of Thr at position 28 and Ala at position 97 is substituted with another amino acid residue in the amino acid sequence represented by SEQ ID NO:25, and a human CDR-grafted antibody composition, wherein the VL of the antibody comprises the amino acid sequence represented by SEQ ID NO:26 or an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26. More preferred are the following antibody compositions: a human CDR-grafted antibody composition, wherein the VH of the antibody comprises an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24; and the VL of the antibody comprises an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26, and a human CDR-grafted antibody composition, wherein the VH of the antibody comprises an amino acid sequence in which at least one amino acid residue selected from the group consisting of Thr at position 28 and Ala at position 97 is substituted with another amino acid residue in the amino acid sequence represented by SEQ ID NO:25; and the VL of the antibody comprises an amino acid sequence in which at least one amino acid residue selected from the group consisting of Ile at position 2, Val at position 3, Gln at position 50 and Val at position 88 is substituted with another amino acid residue in the amino acid sequence represented by SEQ ID NO:26.

A specific example of the human CDR-grafted antibody composition is a human CDR-grafted antibody composition wherein the VH of the antibody comprises an amino acid sequence selected from the group consisting of the amino acid sequences represented by SEQ ID NOs:24, 25, 27, 28, 29, 30, 31 and 32; a human CDR-grafted antibody composition wherein the VL of the antibody comprises an amino acid sequence selected from the group consisting of the amino acid sequences represented by SEQ ID NOs:26, 33, 34 and 35; and a human CDR-grafted antibody composition wherein the VH of the antibody comprises an amino acid sequence selected from the group consisting of the amino acid sequences represented by SEQ ID NOs: 24, 25, 27, 28, 29, 30, 31 and 32, and the VL of the antibody comprises an amino acid sequence selected from the group consisting of the amino acid sequences represented by SEQ ID NOs: 26, 33, 34 and 35.

The human CDR-grated antibody composition of the present invention is most preferably a CDR-grafted antibody composition wherein the VH of the antibody comprises the amino acid sequence represented by SEQ ID NO:27 or 28, and the VL of the antibody comprises the amino acid sequence represented by SEQ ID NO:35.

Also included within the scope of the present invention are antibodies and antibody fragments which specifically bind to an extracellular region of human CCR4 and have no reactivity to a human blood platelet, and consist of amino acid sequences wherein one or more amino acid residues are deleted, added, substituted and/or inserted in the above amino acid sequences.

The number of amino acid residues which are deleted, substituted, inserted and/or added is one or more and is not specifically limited, but it is within the range where deletion, substitution or addition is possible by known methods such as site-directed mutagenesis described in Molecular Cloning, A Laboratory Manual, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc. The suitable number is 1 to dozens, preferably 1 to 20, more preferably 1 to 10, further preferably 1 to 5.

The expression “one or more amino acid residues are deleted, substituted, inserted or added in the amino acid sequence of the antibody composition of the present invention” means that the amino acid sequence of the antibody composition contains deletion, substitution, insertion or addition of a single or plural amino acid residues at a single or plural residues at arbitrary positions therein. Deletion, substitution, insertion and addition may be simultaneously contained in one sequence, and amino acid residues to be substituted, inserted or added may be either natural or not. Examples of the natural amino acid residues are L-alanine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine.

The followings are preferred examples of the amino acid residues capable of mutual substitution. The amino acid residues in the same group shown below can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

The recombinant antibody fragment compositions of the present invention include compositions of antibody fragments which specifically bind to human CCR4 and which contain a part or the whole of the antibody Fc region in which fucose is not bound to the N-acetylglucosamine in the reducing end in complex type N-glycoside-linked sugar chains.

The antibody fragment compositions of the present invention include compositions of antibody fragments, e.g., Fab, Fab′, F(ab′)₂, scFv, diabody, dsFv and a peptide comprising CDR, containing a part or the whole of the antibody Fc region in which fucose is not bound to the N-acetylglucosamine in the reducing end in complex type N-glycoside-linked sugar chains. When the antibody fragment composition does not contain a part or the whole of the antibody Fc region, the antibody fragment may be fused with a part or the whole of the Fc region of the antibody having sugar chains in which fucose is not bound to N-acetylglucosamine in the reducing end in the complex type N-glycoside-linked sugar chains as a fusion protein, or the antibody fragment may be used as a fusion protein composition with a protein comprising a part or the whole of the Fc region.

An Fab fragment is one of the fragments obtained by treatment of IgG with the proteolytic enzyme, papain (cleavage at amino acid residue 224 of H chain). It is an antibody fragment with a molecular weight of approximately 50,000 having antigen-binding activity and composed of the N-terminal half of H chain and the entire L chain linked by a disulfide bond.

The Fab fragment of the present invention can be obtained by treating the antibody composition of the present invention which specifically binds to human CCR4 with the proteolytic enzyme, papain. Alternatively, the Fab fragment may be produced by inserting DNA encoding the Fab fragment of the antibody into an expression vector for prokaryote or eukaryote, and introducing the vector into a prokaryote or eukaryote to induce expression.

An F(ab′)₂ fragment is one of the fragments obtained by treatment of IgG with the proteolytic enzyme, pepsin (cleavage at amino acid residue 234 of H chain). It is an antibody fragment with a molecular weight of approximately 100,000 having antigen-binding activity, which is slightly larger than the Fab fragments linked together by a disulfide bond at the hinge region.

The F(ab′)2 fragment of the present invention can be obtained by treating the antibody composition of the present invention which specifically binds to human CCR4 with the proteolytic enzyme, pepsin. Alternatively, the F(ab′)₂ fragment may be prepared by binding Fab′ fragments described below by a thioether bond or a disulfide bond.

An Fab′ fragment is an antibody fragment with a molecular weight of approximately 50,000 having antigen-binding activity, which is obtained by cleaving the disulfide bond at the hinge region of the above F(ab′)₂ fragment.

The Fab′ fragment of the present invention can be obtained by treating the F(ab′)₂ fragment composition of the present invention which specifically binds to human CCR4 with a reducing agent, dithiothreitol. Alternatively, the Fab′ fragment may be produced by inserting DNA encoding the Fab′ fragment of the antibody into an expression vector for prokaryote or eukaryote, and introducing the vector into a prokaryote or eukaryote to induce expression.

An scFv fragment is a VH-P-VL or VL-P-VH polypeptide in which one VH and one VL are linked via an appropriate peptide linker (hereinafter referred to as P) and which has antigen-binding activity.

The scFv fragment of the present invention can be produced by obtaining cDNAs encoding the VH and VL of the antibody composition of the present invention which specifically binds to human CCR4, constructing DNA encoding the scFv fragment, inserting the DNA into an expression vector for prokaryote or eukaryote, and introducing the expression vector into a prokaryote or eukaryote to induce expression.

A diabody is an antibody fragment which is an scFv dimer showing bivalent antigen binding activity, which may be either monospecific or bispecific.

The diabody of the present invention can be produced by obtaining cDNAs encoding the VH and VL of the antibody composition of the present invention which specifically binds to human CCR4, constructing DNA encoding scFv fragments with P having an amino acid sequence of 8 or less amino acid residues, inserting the DNA into an expression vector for prokaryote or eukaryote, and introducing the expression vector into a prokaryote or eukaryote to induce expression.

A dsFv fragment is an antibody fragment wherein polypeptides in which one amino acid residue of each of VH and VL is substituted with a cysteine residue are linked by a disulfide bond between the cysteine residues. The amino acid residue to be substituted with a cysteine residue can be selected based on antibody tertiary structure prediction according to the method proposed by Reiter, et al. (Protein Engineering, 7, 697-704 (1994)).

The dsFv fragment of the present invention can be produced by obtaining cDNAs encoding the VH and VL of the antibody composition of the present invention which specifically binds to human CCR4, constructing DNA encoding the dsFv fragment, inserting the DNA into an expression vector for prokaryote or eukaryote, and introducing the vector into a prokaryote or eukaryote to induce expression.

A peptide comprising CDR comprises one or more region CDR of VH or VL. A peptide comprising plural CDRs can be prepared by binding CDRs directly or via an appropriate peptide linker.

The peptide comprising CDR of the present invention can be produced by constructing DNA encoding CDR of VH and VL of the antibody composition of the present invention which specifically binds to human CCR4, inserting the DNA into an expression vector for prokaryote or eukaryote, and introducing the expression vector into a prokaryote or eukaryote to induce expression.

The peptide comprising CDR can also be produced by chemical synthesis methods such as the Fmoc method (fluorenylmethyloxycarbonyl method) and the tBoc method (t-butyloxycarbonyl method).

The transformant of the present invention includes any transformant that is obtained by introducing DNA encoding an antibody molecule which specifically binds to human CCR4 into a host cell and that produces the antibody composition of the present invention. Examples of such transformants include those obtained by introducing DNA encoding an antibody molecule which specifically binds to human CCR4 into host cells such as the following (a) or (b):

(a) a cell in which genome is modified so as to have deleted activity of an enzyme relating to the synthesis of an intracellular sugar nucleotide, GDP-fucose;
(b) a cell in which genome is modified so as to have deleted activity of an enzyme relating to the modification of a sugar chain in which 1-position of fucose is bound to 6-position of N-acetylglucosamine in the reducing end through α-bond in a complex type N-glycoside-linked sugar chain.

Specifically, the “modification of genome so as to have deleted activity of an enzyme” refers to introduction of mutation into an expression regulation region of a gene encoding the enzyme so as to delete the expression of the enzyme or introduction of mutation in the amino acid sequence of a gene encoding the enzyme so as to inactivate the enzyme. The “introduction of mutation” refers to carrying out modification of the nucleotide sequence on the genome such as deletion, substitution, insertion and/or addition in the nucleotide sequence. Complete inhibition of the expression or activity of the thus modified genomic gene refers to “knock out of the genomic gene”.

Examples of the enzymes relating to the synthesis of the intracellular sugar nucleotide GDP-fucose include GDP-mannose 4,6-dehydratase (GMD), GDP-4-keto-6-deoxy-D-mannose 3,5-epimerase (Fx) and the like.

Examples of the GDP-mannose 4,6-dehydratase include proteins encoded by the DNAs of the following (a) and (b):

(a) a DNA comprising the nucleotide sequence represented by SEQ ID NO:1;
(b) a DNA which hybridizes with DNA consisting of the nucleotide sequence represented by SEQ ID NO:1 under stringent conditions and which encodes a protein having GDP-mannose 4,6-dehydratase activity.

Examples of the GDP-mannose 4,6-dehydratase also include proteins of the following (a) to (c):

(a) a protein comprising the amino acid sequence represented by SEQ ID NO:2;
(b) a protein consisting of an amino acid sequence wherein one or more amino acid residues are deleted, substituted, inserted and/or added in the amino acid sequence represented by SEQ ID NO:2 and having GDP-mannose 4,6-dehydratase activity;
(c) a protein consisting of an amino acid sequence which has 80% or more homology to the amino acid sequence represented by SEQ ID NO:2 and having GDP-mannose 4,6-dehydratase activity.

Examples of the GDP-4-keto-6-deoxy-D-mannose 3,5-epimerase include proteins encoded by the DNAs of the following (a) and (b):

(a) a DNA comprising the nucleotide sequence represented by SEQ ID NO:3;
(b) a DNA which hybridizes with DNA consisting of the nucleotide sequence represented by SEQ ID NO:3 under stringent conditions and which encodes a protein having GDP-4-keto-6-deoxy-D-mannose 3,5-epimerase activity.

Examples of the GDP-4-keto-6-deoxy-D-mannose 3,5-epimerase also include proteins of the following (a) to (c):

(a) a protein comprising the amino acid sequence represented by SEQ ID NO:4;
(b) a protein consisting of an amino acid sequence wherein one or more amino acid residues are deleted, substituted, inserted and/or added in the amino acid sequence represented by SEQ ID NO:4 and having GDP-4-keto-6-deoxy-D-mannose 3,5-epimerase activity;
(c) a protein consisting of an amino acid sequence which has 80% or more homology to the amino acid sequence represented by SEQ ID NO:4 and having GDP-4-keto-6-deoxy-D-mannose 3,5-epimerase activity.

An example of the enzyme relating to the modification of a sugar chain in which 1-position of fucose is bound to 6-position of N-acetylglucosamine in the reducing end through α-bond in a complex type N-glycoside-linked sugar chain is α1,6-fucosyltransferase.

In the present invention, examples of the α1,6-fucosyltransferase include proteins encoded by the DNAs of the following (a) to (d):

(a) a DNA comprising the nucleotide sequence represented by SEQ ID NO:5;
(b) a DNA comprising the nucleotide sequence represented by SEQ ID NO:6;
(c) a DNA which hybridizes with DNA consisting of the nucleotide sequence represented by SEQ ID NO:5 under stringent conditions and which encodes a protein having α1,6-fucosyltransferase activity;
(d) a DNA which hybridizes with DNA consisting of the nucleotide sequence represented by SEQ ID NO:6 under stringent conditions and which encodes a protein having α1,6-fucosyltransferase activity, or proteins of the following (e) to (j):
(e) a protein comprising the amino acid sequence represented by SEQ ID NO:7;
(f) a protein comprising the amino acid sequence represented by SEQ ID NO:8;
(g) a protein consisting of an amino acid sequence wherein one or more amino acid residues are deleted, substituted, inserted and/or added in the amino acid sequence represented by SEQ ID NO:7 and having α1,6-fucosyltransferase activity;
(h) a protein consisting of an amino acid sequence wherein one or more amino acid residues are deleted, substituted, inserted and/or added in the amino acid sequence represented by SEQ ID NO:8 and having α1,6-fucosyltransferase activity;
(i) a protein consisting of an amino acid sequence which has 80% or more homology to the amino acid sequence represented by SEQ ID NO:7 and having α1,6-fucosyltransferase activity;
(j) a protein consisting of an amino acid sequence which has 80% or more homology to the amino acid sequence represented by SEQ ID NO:8 and having α1,6-fucosyltransferase activity.

The DNAs encoding the amino acid sequences of the enzymes relating to the synthesis of an intracellular sugar nucleotide, GDP-fucose include a DNA comprising the nucleotide sequence represented by SEQ ID NO:1 or 3, and DNA which hybridizes with a DNA comprising the nucleotide sequence represented by SEQ ID NO:1 or 3 under stringent conditions and which encodes a protein having the enzyme activity relating to the synthesis of an intracellular sugar nucleotide, GDP-fucose.

The DNAs encoding the amino acid sequences of α1,6-fucosyltransferase include a DNA comprising the nucleotide sequence represented by SEQ ID NO:5 or 6, and a DNA which hybridizes with DNA comprising the nucleotide sequence represented by SEQ ID NO:5 or 6 under stringent conditions and which encodes a protein having α1,6-fucosyltransferase activity.

In the present invention, the DNA which hybridizes under stringent conditions refers to a DNA which is obtained by colony hybridization, plaque hybridization, Southern hybridization or the like using, for example, a DNA consisting of the nucleotide sequence represented by SEQ ID NO:1, 3, 5 or 6 or a fragment thereof as a probe. A specific example of such DNA is a DNA which can be identified by performing hybridization at 65° C. in the presence of 0.7 to 1.0 M sodium chloride using a filter with colony- or plaque-derived DNA immobilized thereon, and then washing the filter at 65° C. with a 0.1 to 2-fold concentration SSC solution (1-fold concentration SSC solution: 150 mM sodium chloride and 15 mM sodium citrate). Hybridization can be carried out according to the methods described in Molecular Cloning, A Laboratory Manual, Second Edition, Cold Spring Harbor Laboratory Press (1989) (hereinafter referred to as Molecular Cloning, Second Edition); Current Protocols in Molecular Biology, John Wiley & Sons (1987-1997) (hereinafter referred to as Current Protocols in Molecular Biology); DNA Cloning 1: Core Techniques, A Practical Approach, Second Edition, Oxford University (1995), etc. Specifically, the DNA capable of hybridization under stringent conditions includes DNA having at least 60% or more homology, preferably 70% or more homology, more preferably 80% or more homology, further preferably 90% or more homology, particularly preferably 95% or more homology, most preferably 98% or more homology to the nucleotide sequence represented by SEQ ID NO:1, 3, 5 or 6.

In the present invention, the protein consisting of an amino acid sequence wherein one or more amino acid residues are deleted, substituted, inserted and/or added in the amino acid sequence represented by SEQ ID NO:2 or 4 and having the activity of an enzyme relating to the synthesis of an intracellular sugar nucleotide, GDP-fucose, or the protein consisting of an amino acid sequence wherein one or more amino acid residues are deleted, substituted, inserted and/or added in the amino acid sequence represented by SEQ ID NO:7 or 8 and having α1,6-fucosyltransferase activity can be obtained, for example, by introducing a site-directed mutation into DNA having the nucleotide sequence represented by SEQ ID NO:1, 3, 5 or 6 by site-directed mutagenesis described in Molecular Cloning, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc. The number of amino acid residues which are deleted, substituted, inserted and/or added is one or more, and is not specifically limited, but it is within the range where deletion, substitution or addition is possible by known methods such as the above site-directed mutagenesis. The suitable number is 1 to dozens, preferably 1 to 20, more preferably 1 to 10, further preferably 1 to 5.

The protein consisting of an amino acid sequence which has 80% or more homology to the amino acid sequence represented by SEQ ID NO:2, 4, 7 or 8 and having GDP-mannose 4,6-dehydratase activity, GDP-4-keto-6-deoxy-D-mannose 3,5-epimerase activity or α1,6-fucosyltransferase activity includes a protein having at least 80% or more homology, preferably 85% or more homology, more preferably 90% or more homology, further preferably 95% or more homology, particularly preferably 97% or more homology, most preferably 99% or more homology to the amino acid sequence represented by SEQ ID NO:2, 4, 7 or 8, respectively, as calculated by use of analysis software such as BLAST [J. Mol. Biol., 215, 403 (1990)] or FASTA [Methods in Enzymology, 183, 63 (1990)].

The host cell used in the present invention, that is, the host cell in which the activity of an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain is deleted may be obtained by any technique capable of deleting the above enzyme activity. For example, the following techniques can be employed for deleting the above enzyme activity:

(a) gene disruption targeting at a gene encoding the enzyme;
(b) introduction of a dominant-negative mutant of a gene encoding the enzyme;
(c) introduction of a mutation into the enzyme;
(d) inhibition of transcription or translation of a gene encoding the enzyme;
(e) selection of a cell line resistant to a lectin which recognizes a sugar chain structure in which 1-position of fucose is bound to 6-position of N-acetylglucosamine in the reducing end through α-bond in a complex type N-glycoside-linked sugar chain.

As the lectin which recognizes a sugar chain structure in which 1-position of fucose is bound to 6-position of N-acetylglucosamine in the reducing end through α-bond in a complex type N-glycoside-linked sugar chain, any lectin capable of recognizing the sugar chain structure can be used. Specific examples include lentil lectin LCA (lentil agglutinin derived from Lens culinaris), pea lectin PSA (pea lectin derived from Pisum sativum), broad bean lectin VFA (agglutinin derived from Vicia faba), Aleuria aurantia lectin AAL (lectin derived from Aleuria aurantia) and the like.

The “cell resistant to a lectin” refers to a cell in which growth is not inhibited by the presence of a lectin at an effective concentration. The “effective concentration” is a concentration higher than the lowest concentration that does not allow the normal growth of a cell prior to the genome modification (hereinafter referred to also as parent cell line), preferably equal to the lowest concentration that does not allow the normal growth of a cell prior to the genome modification, more preferably 2 to 5 times, further preferably 10 times, most preferably 20 or more times the lowest concentration that does not allow the normal growth of a cell prior to the modification of the genomic gene.

The effective concentration of lectin that does not inhibit growth may be appropriately determined according to each cell line. It is usually 10 μg/ml to 10 mg/ml, preferably 0.5 mg/ml to 2.0 mg/ml.

The host cell for producing the antibody composition of the present invention may be any of the above host cells capable of expressing the antibody composition of the present invention. For example, yeast cells, animal cells, insect cells and plant cells can be used. Examples of the cells include those described in 1 below. Specifically, preferred among animal cells are CHO cell derived from Chinese hamster ovary tissue, rat myeloma cell line YB2/3HL.P2.G11.16Ag.20, mouse myeloma cell line NS0, mouse myeloma cell line SP2/0-Ag14, BHK cell derived from Syrian hamster kidney tissue, an antibody-producing hybridoma cell, human leukemia cell line Namalwa, an embryonic stem cell, and a fertilized egg cell.

A specific example of the transformant of the present invention is Ms705/CCR4, which is a transformant derived from Chinese hamster ovary tissue-derived CHO cell line CHO/DG44 and carrying an introduced gene of the anti-CCR4 antibody of the present invention. The transformant Ms705/CCR4 derived from CHO cell line CHO/DG44 was deposited with International Patent Organism Depositary, National Institute of Advanced Industrial Science and Technology, Central 6, 1, Higashi 1-chome, Tsukuba-shi, Ibaraki, Japan, on Sep. 9, 2003 with accession No. FERM BP-8467.

Described below are the method for preparing a cell producing the antibody composition of the present invention, the method for producing the antibody composition of the present invention, the method for analyzing the antibody composition of the present invention and the method for utilizing the antibody composition of the present invention.

### 1. Preparation of a Cell Producing the Antibody Composition of the Present Invention

The cell producing the antibody composition of the present invention (hereinafter referred to as the cell of the present invention) can be prepared by preparing a host cell used for the production of the antibody composition of the present invention by the following techniques and then introducing a gene encoding the anti-human CCR4 antibody into the host cell by the method described in 2 below.

### (1) Gene Disruption Technique Targeting at a Gene Encoding an Enzyme

The host cell used for the production of the antibody composition of the present invention can be prepared by a gene disruption technique targeting a gene encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain. Examples of the enzymes relating to the synthesis of an intracellular sugar nucleotide, GDP-fucose include GDP-mannose 4,6-dehydratase (hereinafter referred to as GMD) and GDP-4-keto-6-deoxy-D-mannose 3,5-epimerase (hereinafter referred to as Fx). Examples of the enzymes relating to the modification of a sugar chain in which 1-position of fucose is bound to 6-position of N-acetylglucosamine in the reducing end through α-bond in a complex type N-glycoside-linked sugar chain include α1,6-fucosyltransferase and α-L-fucosidase.

The gene as used herein includes DNA and RNA.

The method of gene disruption may be any method capable of disrupting the gene encoding the target enzyme. Useful methods include the antisense method, the ribozyme method, the homologous recombination method, the RNA-DNA oligonucleotide method (hereinafter referred to as the RDO method), the RNA interference method (hereinafter referred to as the RNAi method), the method using a retrovirus and the method using a transposon. These methods are specifically described below.

### (a) Preparation of the Host Cell for the Production of the Antibody Composition of the Present Invention by the Antisense Method or the Ribozyme Method

The host cell used for the production of the antibody composition of the present invention can be prepared by the antisense method or the ribozyme method described in Cell Technology, 12, 239 (1993); BIO/TECHNOLOGY, 17, 1097 (1999); Hum. Mol. Genet., 5, 1083 (1995); Cell Technology, 13, 255 (1994); Proc. Natl. Acad. Sci. U.S.A., 96, 1886 (1999); etc. targeting at a gene encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain, for example, in the following manner.

A cDNA or a genomic DNA encoding an enzyme relating to the synthesis of the intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain is prepared.

The nucleotide sequence of the prepared cDNA or genomic DNA is determined.

Based on the determined DNA sequence, an antisense gene or a ribozyme of appropriate length is designed which comprises a DNA moiety encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, non-translated regions and introns.

In order to express the antisense gene or ribozyme in a cell, a recombinant vector is prepared by inserting a fragment or full-length of the prepared DNA into a site downstream of a promoter in an appropriate expression vector.

The recombinant vector is introduced into a host cell suited for the expression vector to obtain a transformant.

The host cell used for the production of the antibody composition of the present invention can be obtained by selecting a transformant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. The host cell used for the production of the antibody composition of the present invention can also be obtained by selecting a transformant using, as a marker, the sugar chain structure of a glycoprotein on the cell membrane or the sugar chain structure of the produced antibody molecule.

As the host cell used for the production of the antibody composition of the present invention, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below.

The expression vectors that can be employed are those capable of autonomous replication or integration into the chromosome in the above host cells and comprising a promoter at a position appropriate for the transcription of the designed antisense gene or ribozyme. Examples of the expression vectors include those described in 2 below.

Introduction of a gene into various host cells can be carried out by the methods suitable for introducing a recombinant vector into various host cells described in 2 below.

Selection of a transformant using, as a marker, the activity of an enzyme relating to the synthesis of an intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain can be carried out, for example, by the following methods.

### Methods for Selecting a Transformant

A cell in which the activity of an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain is deleted can be selected by measuring the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain using biochemical methods or genetic engineering techniques described in Shin Seikagaku Jikken Koza (New Lectures on Experiments in Biochemistry) 3—Saccharides I, Glycoprotein (Tokyo Kagaku Dojin), edited by The Japanese Biochemical Society (1988); Cell Technology, Extra Edition, Experimental Protocol Series, Glycobiology Experimental Protocol, Glycoprotein, Glycolipid and Proteoglycan (Shujunsha), edited by Naoyuki Taniguchi, Akemi Suzuki, Kiyoshi Furukawa and Kazuyuki Sugawara (1996); Molecular Cloning, Second Edition; Current Protocols in Molecular Biology; and the like. An example of the biochemical methods is a method in which the enzyme activity is evaluated using an enzyme-specific substrate. Examples of the genetic engineering techniques include Northern analysis and RT-PCR in which the amount of mRNA for a gene encoding the enzyme is measured.

Selection of a transformant using, as a marker, the sugar chain structure of a glycoprotein on the cell membrane can be carried out, for example, by the method described in 1(5) below. Selection of a transformant using, as a marker, the sugar chain structure of a produced antibody molecule can be carried out, for example, by the methods described in 4 or 5 below.

Preparation of a cDNA encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain can be carried out, for example, by the following method.

Preparation of cDNA

Total RNA or mRNA is prepared from a various host cell tissue or cell.

A cDNA library is prepared from the total RNA or mRNA.

Degenerative primers are prepared based on the amino acid sequence of an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain, and a gene fragment encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain is obtained by PCR using the prepared cDNA library as a template.

A DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be obtained by screening the cDNA library using the obtained gene fragment as a probe.

As the mRNA of a human or non-human animal tissue or cell, commercially available one (for example, manufactured by Clontech) may be use, or it may be prepared from a human or non-human animal tissue or cell in the following manner.

The methods for preparing total RNA from a human or non-human animal tissue or cell include the guanidine thiocyanate-cesium trifluoroacetate method [Methods in Enzymology, 154, 3 (1987)], the acidic guanidine thiocyanate-phenol-chloroform (AGPC) method [Analytical Biochemistry, 162, 156 (1987); Experimental Medicine, 9, 1937 (1991)] and the like.

The methods for preparing mRNA as poly(A)⁺RNA from the total RNA include the oligo (dT) immobilized cellulose column method (Molecular Cloning, Second Edition).

It is also possible to prepare mRNA by using a commercially available kit such as Fast Track mRNA Isolation Kit™ (manufactured by Invitrogen) or Quick Prep mRNA Purification Kit™ (manufactured by Pharmacia).

A cDNA library is prepared from the obtained mRNA of a human or non-human animal tissue or cell. The methods for preparing the cDNA library include the methods described in Molecular Cloning, Second Edition; Current Protocols in Molecular Biology; A Laboratory Manual, 2nd Ed. (1989); etc., and methods using commercially available kits such as SuperScript Plasmid System for cDNA Synthesis and Plasmid Cloning™ (manufactured by Life Technologies) and ZAP-cDNA Synthesis Kit™ (manufactured by STRATAGENE).

As the cloning vector for preparing the cDNA library, any vectors, e.g. phage vectors and plasmid vectors, can be used so long as they are autonomously replicable in Escherichia coli K12. Examples of suitable vectors include ZAP Express™ [manufactured by STRATAGENE; Strategies, 5, 58 (1992)], pBluescript II SK(+) [Nucleic Acids Research, 17, 9494 (1989)], λZAP II™ (manufactured by STRATAGENE), λgt10, λgt11 [DNA Cloning, A Practical Approach, 1, 49 (1985)], λTriplEx™ (manufactured by Clontech), λExCell™ (manufactured by Pharmacia), pT7T318U™ (manufactured by Pharmacia), pcD2 [Mol Cell Biol., 3, 280 (1983)] and pUC18 [Gene, 33, 103 (1985)].

Any microorganism can be used as the host microorganism for preparing the cDNA library, but Escherichia coli is preferably used. Examples of suitable host microorganisms are Escherichia coli XL1-Blue MRF′™ [manufactured by STRATAGENE; Strategies, 5, 81 (1992)], Escherichia coli C600 [Genetics, 39, 440 (1954)], Escherichia coli Y1088 [Science, 222, 778 (1983)], Escherichia coli Y1090 [Science, 222, 778 (1983)], Escherichia coli NM522 [J. Mol. Biol., 166, 1 (1983)], Escherichia coli K802 [J. Mol. Biol., 16, 118 (1966)] and Escherichia coli JM105 [Gene, 38, 275 (1985)].

The cDNA library may be used as such in the following analysis. Alternatively, in order to efficiently obtain full-length cDNAs by decreasing the ratio of partial cDNAs, a cDNA library prepared using the oligo-cap method developed by Sugano, et al. [Gene, 138, 171 (1994); Gene, 200, 149 (1997); Protein, Nucleic Acid and Enzyme, 41, 603 (1996); Experimental Medicine, 11, 2491 (1993); cDNA Cloning (Yodosha) (1996); Methods for Preparing Gene Libraries (Yodosha) (1994)] may be used in the following analysis.

Degenerative primers specific for the 5′-terminal and 3′-terminal nucleotide sequences of a nucleotide sequence presumed to encode the amino acid sequence of an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain are prepared based on the amino acid sequence of the enzyme. A gene fragment encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be obtained by DNA amplification by PCR [PCR Protocols, Academic Press (1990)] using the prepared cDNA library as a template.

It can be confirmed that the obtained gene fragment is a cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain by analyzing the nucleotide sequence by generally employed methods such as the dideoxy method of Sanger, et al. [Proc. Natl. Acad. Sci. U.S.A., 74, 5463 (1977)] or by use of nucleotide sequencers such as ABI PRISM 377 DNA Sequencer (manufactured by Applied Biosystems).

A DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be obtained from the cDNA or cDNA library synthesized from the mRNA contained in a human or non-human animal tissue or cell by colony hybridization or plaque hybridization (Molecular Cloning, Second Edition) using the above gene fragment as a probe.

A cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can also be obtained by amplification by PCR using the cDNA or cDNA library synthesized from the mRNA contained in a human or non-human animal tissue or cell as a template and using the primers used for obtaining the gene fragment encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain.

The nucleotide sequence of the obtained cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be determined by generally employed sequencing methods such as the dideoxy method of Sanger, et al. [Proc. Natl. Acad. Sci. U.S.A., 74, 5463 (1977)] or by use of nucleotide sequencers such as ABI PRISM 377 DNA Sequencer (manufactured by Applied Biosystems).

By carrying out a search of nucleotide sequence databases such as GenBank, EMBL or DDBJ using a homology search program such as BLAST based on the determined nucleotide sequence of the cDNA, it can be confirmed that the obtained DNA is a gene encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain among the genes in the nucleotide sequence database.

Examples of the nucleotide sequences of the genes encoding the enzyme relating to the synthesis of an intracellular sugar nucleotide, GDP-fucose obtained by the above methods include the nucleotide sequences represented by SEQ ID NO:1 or 3.

Examples of the nucleotide sequences of the genes encoding the enzyme relating to the modification of a sugar chain in which 1-position of fucose is bound to 6-position of N-acetylglucosamine in the reducing end through α-bond in a complex type N-glycoside-linked sugar chain obtained by the above methods include the nucleotide sequences represented by SEQ ID NO:5 or 6.

The cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can also be obtained by chemical synthesis with a DNA synthesizer such as DNA Synthesizer Model 392 (manufactured by Perkin Elmer) utilizing the phosphoramidite method based on the determined nucleotide sequence of the DNA.

Preparation of a genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be carried out, for example, by the following method.

### Method for Preparing Genomic DNA

The genomic DNA can be prepared by known methods described in Molecular Cloning, Second Edition; Current Protocols in Molecular Biology; etc. In addition, the genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be obtained by using a kit such as Genomic DNA Library Screening System™ (manufactured by Genome Systems) or Universal GenomeWalker™ Kits (manufactured by CLONTECH).

The nucleotide sequence of the obtained DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be determined by generally employed sequencing methods such as the dideoxy method of Sanger, et al. [Proc. Natl. Acad. Sci. U.S.A., 74, 5463 (1977)] or by use of nucleotide sequencers such as ABI PRISM 377 DNA Sequencer (manufactured by Applied Biosystems).

By carrying out a search of nucleotide sequence databases such as GenBank, EMBL or DDBJ using a homology search program such as BLAST based on the determined nucleotide sequence of the genomic DNA, it can be confirmed that the obtained DNA is a gene encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain among the genes in the nucleotide sequence database.

The genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can also be obtained by chemical synthesis with a DNA synthesizer such as DNA Synthesizer Model 392 (manufactured by Perkin Elmer) utilizing the phosphoamidite method based on the determined nucleotide sequence of the DNA.

Examples of the nucleotide sequences of the genomic DNAs encoding the enzyme relating to the synthesis of an intracellular sugar nucleotide, GDP-fucose obtained by the above methods include the nucleotide sequences represented by SEQ ID NOs:9, 10, 11 and 12.

An example of the nucleotide sequence of the genomic DNA encoding the enzyme relating to the modification of a sugar chain in which 1-position of fucose is bound to 6-position of N-acetylglucosamine in the reducing end through α-bond in a complex type N-glycoside-linked sugar chain obtained by the above methods is the nucleotide sequence represented by SEQ ID NO:13.

The host cell used for the production of the antibody composition of the present invention can also be obtained without using an expression vector by directly introducing into a host cell an antisense oligonucleotide or ribozyme designed based on the nucleotide sequence encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain.

The antisense oligonucleotide or ribozyme can be prepared by known methods or by using a DNA synthesizer. Specifically, based on the sequence information on an oligonucleotide having a sequence corresponding to 5 to 150, preferably 5 to 60, more preferably 10 to 40 contiguous nucleotides in the nucleotide sequence of the cDNA or genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, an oligonucleotide corresponding to the sequence complementary to the above oligonucleotide (antisense oligonucleotide) or a ribozyme comprising the oligonucleotide sequence can be synthesized.

The oligonucleotide includes oligo RNA and derivatives of the oligonucleotide (hereinafter referred to as oligonucleotide derivatives).

The oligonucleotide derivatives include an oligonucleotide derivative wherein the phosphodiester bond in the oligonucleotide is converted to a phosophorothioate bond, an oligonucleotide derivative wherein the phosphodiester bond in the oligonucleotide is converted to an N3′-P5′ phosphoamidate bond, an oligonucleotide derivative wherein the ribose-phosphodiester bond in the oligonucleotide is converted to a peptide-nucleic acid bond, an oligonucleotide derivative wherein the uracil in the oligonucleotide is substituted with C-5 propynyluracil, an oligonucleotide derivative wherein the uracil in the oligonucleotide is substituted with C-5 thiazolyluracil, an oligonucleotide derivative wherein the cytosine in the oligonucleotide is substituted with C-5 propynylcytosine, an oligonucleotide derivative wherein the cytosine in the oligonucleotide is substituted with phenoxazine-modified cytosine, an oligonucleotide derivative wherein the ribose in the oligonucleotide is substituted with 2′-O-propylribose, and an oligonucleotide derivative wherein the ribose in the oligonucleotide is substituted with 2′-methoxyethoxyribose [Cell Technology, 16, 1463 (1997)].

### (b) Preparation of the Host Cell for the Production of the Antibody Composition of the Present Invention by the Homologous Recombination Method

The host cell used for the production of the antibody composition of the present invention can be prepared by modifying a target gene on the chromosome by the homologous recombination method targeting a gene encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain.

Modification of the target gene on the chromosome can be carried out by using the methods described in Manipulating the Mouse Embryo, A Laboratory Manual, Second Edition, Cold Spring Harbor Laboratory Press (1994) (hereinafter referred to as Manipulating the Mouse Embryo, A Laboratory Manual); Gene Targeting, A Practical Approach, IRL Press at Oxford University Press (1993); Biomanual Series 8, Gene Targeting, Preparation of Mutant Mice Using ES Cells, Yodosha (1995) (hereinafter referred to as Preparation of Mutant Mice Using ES Cells); etc., for example, in the following manner.

A genomic DNA encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain is prepared.

Based on the nucleotide sequence of the genomic DNA, a target vector is prepared for homologous recombination of a target gene to be modified (e.g., the structural gene or promoter gene for the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain).

The host cell used for the preparation of the cell of the present invention can be prepared by introducing the prepared target vector into a host cell and selecting a cell in which homologous recombination occurred between the target gene on the chromosome and the target vector.

As the host cell, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below.

The genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be prepared by the methods for preparing a genomic DNA described in the above 1 (1) (a), etc.

Examples of the nucleotide sequences of the genomic DNAs encoding the enzyme relating to the synthesis of the intracellular sugar nucleotide GDP-fucose obtained by the above methods include the nucleotide sequences represented by SEQ ID NOs:9, 10, 11 and 12.

An example of the nucleotide sequence of the genomic DNA encoding the enzyme relating to the modification of a sugar chain in which 1-position of fucose is bound to 6-position of N-acetylglucosamine in the reducing end through α-bond in a complex type N-glycoside-linked sugar chain obtained by the above methods is the nucleotide sequence represented by SEQ ID NO:13.

The target vector for use in the homologous recombination of the target gene on the chromosome can be prepared according to the methods described in Gene Targeting, A Practical Approach, IRL Press at Oxford University Press (1993); Preparation of Mutant Mice Using ES Cells; etc. The target vector may be either a replacement-type one or an insertion-type one.

Introduction of the target vector into various host cells can be carried out by the methods suitable for introducing a recombinant vector into various host cells described in 3 below.

The methods for efficiently selecting a homologous recombinant include positive selection, promoter selection, negative selection and polyA selection described in Gene Targeting, A Practical Approach, IRL Press at Oxford University Press (1993); Preparation of Mutant Mice Using ES Cells; etc. The methods for selecting the desired homologous recombinant from the selected cell lines include Southern hybridization (Molecular Cloning, Second Edition) and PCR [PCR Protocols, Academic Press (1990)] with the genomic DNA.

### (c) Preparation of the Host Cell for the Production of the Antibody Composition of the Present Invention by the RDO Method

The host cell used for the production of the antibody composition of the present invention can be prepared by the RDO method targeting a gene encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain, for example, in the following manner.

A cDNA or a genomic DNA encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain is prepared by the methods described in the above 1 (1) (a).

The nucleotide sequence of the prepared cDNA or genomic DNA is determined.

Based on the determined DNA sequence, an RDO construct of appropriate length which comprises a DNA moiety encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, non-translated regions and introns is designed and synthesized.

The host cell of the present invention can be obtained by introducing the synthesized RDO into a host cell and then selecting a transformant in which a mutation occurred in the target enzyme, that is, the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain.

As the host cell, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below.

Introduction of the RDO into various host cells can be carried out by the methods suitable for introducing a recombinant vector into various host cells described in 2 below.

The cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be prepared by the methods for preparing a cDNA described in the above 1 (1) (a) or the like.

The genomic DNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be prepared by the methods for preparing a genomic DNA described in the above 1 (1) (b) or the like.

After DNA is cleaved with appropriate restriction enzymes, the nucleotide sequence of the DNA can be determined by cloning the DNA fragments into a plasmid such as pBluescript SK(−)™ (manufactured by Stratagene), subjecting the clones to the reaction generally used as a method for analyzing a nucleotide sequence such as the dideoxy method of Sanger et al. [Proc. Natl. Acad. Sci. USA, 74, 5463 (1977)] or the like, and then analyzing the clones by using an automatic nucleotide sequence analyzer such as ABI PRISM 377 DNA Sequencer (manufactured by Applied Biosystems) or the like.

The RDO can be prepared by conventional methods or by using a DNA synthesizer.

The methods for selecting a cell in which a mutation occurred by introducing the RDO into the host cell, in the gene encoding the target enzyme, that is, the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods for directly detecting mutations in chromosomal genes described in Molecular Cloning, Second Edition; Current Protocols in Molecular Biology; etc.

For the selection of the transformant, the following methods can also be employed: the method using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain described in the above 1 (1) (a); the method using, as a marker, the sugar chain structure of a glycoprotein on the cell membrane described in 1 (5) below; and the method using, as a marker, the sugar chain structure of a produced antibody molecule described in 4 and 5 below.

The RDO can be designed according to the descriptions in Science, 273, 1386 (1996); Nature Medicine, 4, 285 (1998); Hepatology, 25, 1462 (1997); Gene Therapy, 5, 1960 (1999); Gene Therapy, 5, 1960 (1999); J. Mol. Med., 75, 829 (1997); Proc. Natl. Acad. Sci. USA, 96, 8774 (1999); Proc. Natl. Acad. Sci. USA, 96, 8768 (1999); Nuc. Acids Res., 27, 1323 (1999); Invest. Dermatol., 111, 1172 (1998); Nature Biotech., 16, 1343 (1998); Nature Biotech., 18, 43 (2000); Nature Biotech., 18, 555 (2000); etc.

### (d) Preparation of the Host Cell for the Production of the Antibody Composition of the Present Invention by the RNAi Method

The host cell used for the production of the antibody composition of the present invention can be prepared by the RNAi method targeting a gene encoding an enzyme relating to the synthesis of an intracellular sugar nucleotide, GDP-fucose or an enzyme relating to the modification of a sugar chain in which 1-position of fucose is bound to 6-position of N-acetylglucosamine in the reducing end through α-bond in a complex type N-glycoside-linked sugar chain, for example, in the following manner.

A cDNA encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain is prepared by the methods described in the above 1 (1) (a).

The nucleotide sequence of the prepared cDNA is determined.

Based on the determined cDNA sequence, an RNAi gene of appropriate length is designed which comprises a moiety encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, or non-translated regions.

In order to express the RNAi gene in a cell, a recombinant vector is prepared by inserting a fragment or full-length of the prepared cDNA into a site downstream of a promoter in an appropriate expression vector.

The recombinant vector is introduced into a host cell suited for the expression vector to obtain a transformant.

The host cell used for the preparation of the cell of the present invention can be obtained by selecting a transformant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, or the sugar chain structure of a produced antibody molecule or a glycoprotein on the cell membrane.

As the host cell, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below.

The expression vectors that can be employed are those capable of autonomous replication or integration into the chromosome in the above host cells and comprising a promoter at a position appropriate for the transcription of the designed RNAi gene. Examples of the expression vectors include those described in 2 below.

Introduction of a gene into various host cells can be carried out by the methods suitable for introducing a recombinant vector into various host cells described in 2 below.

The methods for selecting the transformant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods described in the above 1 (1) (a).

The methods for selecting the transformant using, as a marker, the sugar chain structure of a glycoprotein on the cell membrane include the method described in 1 (5). The methods for selecting the transformant using, as a marker, the sugar chain structure of a produced antibody molecule include the methods described in 4 or 5 below.

The cDNA encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain can be prepared by the methods for preparing a cDNA described in the above 1 (1) (a), etc.

The host cell used for the preparation of the cell of the present invention can also be obtained without using an expression vector by directly introducing into a host cell the RNAi gene designed based on the nucleotide sequence encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain.

The RNAi gene can be prepared by known methods or by using a DNA synthesizer.

The RNAi gene construct can be designed according to the descriptions in Nature, 391, 806 (1998); Proc. Natl. Acad. Sci. USA, 95, 15502 (1998); Nature, 395, 854 (1998); Proc. Natl. Acad. Sci. USA, 96, 5049 (1999); Cell, 95, 1017 (1998); Proc. Natl. Acad. Sci. USA, 96, 1451 (1999); Proc. Natl. Acad. Sci. USA, 95, 13959 (1998); Nature Cell Biol., 2, 70 (2000); etc.

### (e) Preparation of the Host Cell for the Production of the Antibody Composition of the Present Invention by the Method Using a Transposon

The host cell used for the production of the antibody composition of the present invention can be prepared by using the transposon system described in Nature Genet., 25, 35 (2000), etc., and then selecting a mutant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, or the sugar chain structure of a produced antibody molecule or a glycoprotein on the cell membrane.

The transposon system is a system for inducing a mutation by random insertion of an exogenous gene into the chromosome, wherein usually an exogenous gene inserted into a transposon is used as a vector for inducing a mutation and a transposase expression vector for randomly inserting the gene into the chromosome is introduced into the cell at the same time.

Any transposase can be used so long as it is suitable for the sequence of the transposon to be used.

As the exogenous gene, any gene can be used so long as it can induce a mutation in the DNA of a host cell.

As the host cell, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below. Introduction of the gene into various host cells can be carried out by the methods suitable for introducing a recombinant vector into various host cells described in 2 below.

The methods for selecting the mutant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods described in the above 1 (1) (a).

The methods for selecting the mutant using, as a marker, the sugar chain structure of a glycoprotein on the cell membrane include the method described in 1 (5). The methods for selecting the mutant using, as a marker, the sugar chain structure of a produced antibody molecule include the methods described in 4 or 5 below.

### (2) Technique of Introducing a Dominant-Negative Mutant of a Gene Encoding an Enzyme

The host cell used for the production of the antibody composition of the present invention can be prepared by using the method of introducing a dominant-negative mutant of a target gene, i.e., a gene encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain. Examples of the enzymes relating to the synthesis of the intracellular sugar nucleotide GDP-fucose include GMD and Fx. Examples of the enzymes relating to the modification of a sugar chain in which 1-position of fucose is bound to 6-position of N-acetylglucosamine in the reducing end through α-bond in a complex type N-glycoside-linked sugar chain include α1,6-fucosyltransferase and α-L-fucosidase.

These enzymes have substrate specificity and catalyze specific reactions. By disrupting the active center of such enzymes having substrate specificity and catalytic action, their dominant-negative mutants can be prepared. Preparation of a dominant-negative mutant is described in detail below, using for an example GMD among the target enzymes.

As a result of the analysis of the tertiary structure of GMD derived from Escherichia coli, it has been revealed that four amino acids (threonine at position 133, glutamic acid at position 135, tyrosine at position 157 and lysine at position 161) have an important function for the enzyme activity (Structure, 8, 2, 2000). That is, the mutants prepared by substituting the above four amino acids by other amino acids based on the tertiary structure information all showed significantly decreased enzyme activity. On the other hand, little change was observed in the ability of the mutants to bind to the GMD coenzyme NADP or the substrate GDP-mannose. Accordingly, a dominant-negative mutant can be prepared by substituting the four amino acids which are responsible for the enzyme activity of GMD. On the basis of the result of preparation of a dominant-negative mutant of GMD derived from Escherichia coli, dominant-negative mutants of other GMDs can be prepared by performing homology comparison and tertiary structure prediction using the amino acid sequence information. For example, in the case of GMD derived from CHO cell (SEQ ID NO:2), a dominant-negative mutant can be prepared by substituting threonine at position 155, glutamic acid at position 157, tyrosine at position 179 and lysine at position 183 by other amino acids. Preparation of such a gene carrying introduced amino acid substitutions can be carried out by site-directed mutagenesis described in Molecular Cloning, Second Edition; Current Protocols in Molecular Biology; etc.

The host cell used for the production of the antibody composition of the present invention can be prepared according to the method of gene introduction described in Molecular Cloning, Second Edition; Current Protocols in Molecular Biology; Manipulating the Mouse Embryo, Second Edition; etc. using a gene encoding a dominant-negative mutant of a target enzyme (hereinafter abbreviated as dominant-negative mutant gene) prepared as above, for example, in the following manner.

A dominant-negative mutant gene encoding the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain is prepared.

Based on the full-length DNA of the prepared dominant-negative mutant gene, a DNA fragment of appropriate length containing a region encoding the protein is prepared according to need.

A recombinant vector is prepared by inserting the DNA fragment or full-length DNA into a site downstream of a promoter in an appropriate expression vector.

The recombinant vector is introduced into a host cell suited for the expression vector to obtain a transformant.

The host cell used for the preparation of the cell of the present invention can be obtained by selecting a transformant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain, or the sugar chain structure of a produced antibody molecule or a glycoprotein on the cell membrane.

As the host cell, any yeast cell, animal cell, insect cell, plant cell, or the like can be used so long as it has a gene encoding the target enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain. Examples of the host cells include those described in 2 below.

The expression vectors that can be employed are those capable of autonomous replication or integration into the chromosome in the above host cells and comprising a promoter at a position appropriate for the transcription of the DNA encoding the desired dominant-negative mutant. Examples of the expression vectors include those described in 2 below.

Introduction of a gene into various host cells can be carried out by the methods suitable for introducing a recombinant vector into various host cells described in 2 below.

The methods for selecting the transformant using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods described in the above 1 (1) (a).

The methods for selecting the transformant using, as a marker, the sugar chain structure of a glycoprotein on the cell membrane include the method described in 1 (5) below. The methods for selecting the transformant using, as a marker, the sugar chain structure of a produced antibody molecule include the methods described in 4 or 5 below.

(3) Technique of Introducing a Mutation into an Enzyme

The host cell used for the production of the antibody composition of the present invention can be prepared by introducing a mutation into a gene encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain, and then selecting a desired cell line in which the mutation occurred in the enzyme.

Examples of the enzymes relating to the synthesis of the intracellular sugar nucleotide GDP-fucose include GMD and Fx. Examples of the enzymes relating to the modification of a sugar chain in which 1-position of fucose is bound to 6-position of N-acetylglucosamine in the reducing end through α-bond in a complex type N-glycoside-linked sugar chain include α1,6-fucosyltransferase and α-L-fucosidase.

The methods for introducing a mutation into the enzyme include: 1) a method in which a desired cell line is selected from mutants obtained by subjecting a parent cell line to mutagenesis or by spontaneous mutation using, as a marker, the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain; 2) a method in which a desired cell line is selected from mutants obtained by subjecting a parent cell line to mutagenesis or by spontaneous mutation using, as a marker, the sugar chain structure of a produced antibody molecule; and 3) a method in which a desired cell line is selected from mutants obtained by subjecting a parent cell line to mutagenesis or by spontaneous mutation using, as a marker, the sugar chain structure of a glycoprotein on the cell membrane.

Mutagenesis may be carried out by any method capable of inducing a point mutation, a deletion mutation or a frameshift mutation in DNA of a cell of a parent cell line.

Suitable methods include treatment with ethyl nitrosourea, nitrosoguanidine, benzopyrene or an acridine dye and radiation treatment. Various alkylating agents and carcinogens are also useful as mutagens. A mutagen is allowed to act on a cell by the methods described in Soshiki Baiyo no Gijutsu (Tissue Culture Techniques), Third Edition (Asakura Shoten), edited by The Japanese Tissue Culture Association (1996); Nature Genet., 24, 314 (2000); etc.

Examples of the mutants generated by spontaneous mutation include spontaneous mutants obtained by continuing subculture under usual cell culture conditions without any particular treatment for mutagenesis.

The methods for measuring the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods described in the above 1 (1) (a). The methods for determining the sugar chain structure of a produced antibody molecule include the methods described in 4 or 5 below. The methods for determining the sugar chain structure of a glycoprotein on the cell membrane include the method described in 1 (5).

### (4) Technique of Suppressing Transcription or Translation of a Gene Encoding an Enzyme

The host cell used for the production of the antibody composition of the present invention can be prepared by inhibiting transcription or translation of a target gene, i.e., a gene encoding an enzyme relating to the synthesis of the intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain using the antisense RNA/DNA technique [Bioscience and Industry, 50, 322 (1992); Chemistry, 46, 681 (1991); Biotechnology, 9, 358 (1992); Trends in Biotechnology, 10, 87 (1992); Trends in Biotechnology, 10, 152 (1992); Cell Technology, 16, 1463 (1997)], the triple helix technique [Trends in Biotechnology, 10, 132 (1992)], etc.

Examples of the enzymes relating to the synthesis of the intracellular sugar nucleotide GDP-fucose include GMD and Fx. Examples of the enzymes relating to the modification of a sugar chain in which 1-position of fucose is bound to 6-position of N-acetylglucosamine in the reducing end through α-bond in a complex type N-glycoside-linked sugar chain include α1,6-fucosyltransferase and α-L-fucosidase.

The methods for measuring the activity of the enzyme relating to the synthesis of an intracellular sugar nucleotide, GDP-fucose or the enzyme relating to the modification of a sugar chain in which 1-position of fucose is bound to 6-position of N-acetylglucosamine in the reducing end through α-bond in a complex type N-glycoside-linked sugar chain include the methods described in the above 1 (1) (a).

The methods for determining the sugar chain structure of a glycoprotein on the cell membrane include the method described in 1 (5). The methods for determining the sugar chain structure of a produced antibody molecule include the methods described in 4 or 5 below.

(5) Technique of Selecting a Cell Line Resistant to a Lectin which Recognizes a Sugar Chain Structure in which 1-Position of Fucose is Bound to 6-Position of N-Acetylglucosamine in The Reducing End Through α-Bond in a Complex Type N-Glycoside-Linked Sugar Chain

The host cell used for the production of the antibody composition of the present invention can be prepared by selecting a cell line resistant to a lectin which recognizes a sugar chain structure in which 1-position of fucose is bound to 6-position of N-acetylglucosamine in the reducing end through α-bond in a complex type N-glycoside-linked sugar chain.

Selection of a cell line resistant to a lectin which recognizes a sugar chain structure in which 1-position of fucose is bound to 6-position of N-acetylglucosamine in the reducing end through α-bond in a complex type N-glycoside-linked sugar chain can be carried out, for example, by the method using a lectin described in Somatic Cell Mol. Genet., 12, 51 (1986), etc.

As the lectin, any lectin can be used so long as it recognizes a sugar chain structure in which 1-position of fucose is bound to 6-position of N-acetylglucosamine in the reducing end through α-bond in a complex type N-glycoside-linked sugar chain. Specific examples include lentil lectin LCA (lentil agglutinin derived from Lens culinaris), pea lectin PSA (pea lectin derived from Pisum sativum), broad bean lectin VFA (agglutinin derived from Vicia faba) and Aleuria aurantia lectin AAL (lectin derived from Aleuria aurantia).

Specifically, the cell line of the present invention resistant to a lectin which recognizes a sugar chain structure in which 1-position of fucose is bound to 6-position of N-acetylglucosamine in the reducing end through α-bond in a complex type N-glycoside-linked sugar chain can be selected by culturing cells in a medium containing the above lectin at a concentration of 1 μg/ml to 1 mg/ml for one day to 2 weeks, preferably one day to one week, subculturing surviving cells or picking up a colony and transferring it into a culture vessel, and subsequently continuing the culturing using the medium containing the lectin.

### 2. Process for Producing the Antibody Composition

The antibody composition of the present invention can be obtained by expressing it in a host cell using the methods described in Molecular Cloning, Second Edition; Current Protocols in Molecular Biology; Antibodies, A Laboratory Manual, Cold Spring Harbor Laboratory, 1988 (hereinafter referred to as Antibodies); Monoclonal Antibodies: Principles and Practice, Third Edition, Acad. Press, 1993 (hereinafter referred to as Monoclonal Antibodies); Antibody Engineering, A Practical Approach, IRL Press at Oxford University Press, 1996 (hereinafter referred to as Antibody Engineering); etc., for example, in the following manner.

A full-length cDNA encoding an anti-human CCR4 antibody molecule is prepared, and a DNA fragment of appropriate length comprising a region encoding the antibody molecule is prepared.

A recombinant vector is prepared by inserting the DNA fragment or full-length DNA into a site downstream of a promoter in an appropriate expression vector.

The recombinant vector is introduced into a host cell suited for the expression vector to obtain a transformant producing the antibody molecule.

As the host cell, any yeast cells, animal cells, insect cells, plant cells, etc. that are capable of expressing the desired gene can be used.

Also useful are cells obtained by selecting cells in which the activity of an enzyme relating to the modification of an N-glycoside-linked sugar chain bound to the Fc region of an antibody molecule, i.e., an enzyme relating to the synthesis of an intracellular sugar nucleotide GDP-fucose or an enzyme relating to the modification of a sugar chain in which 1-position of fucose is bound to 6-position of N-acetylglucosamine in the reducing end through α-bond in a complex type N-glycoside-linked sugar chain is deleted, and cells obtained by various artificial techniques described in the above 1.

The expression vectors that can be employed are those capable of autonomous replication or integration into the chromosome in the above host cells and comprising a promoter at a position appropriate for the transcription of the DNA encoding the desired antibody molecule.

The cDNA can be prepared from a human or non-human animal tissue or cell according to the methods for preparing a cDNA described in the above 1 (1) (a) using, e.g., a probe or primers specific for the desired antibody molecule.

When yeast is used as the host cell, YEP13 (ATCC 37115), YEp24 (ATCC 37051), YCp50 (ATCC 37419), etc. can be used as the expression vector.

As the promoter, any promoters capable of expressing in yeast strains can be used. Suitable promoters include promoters of genes of the glycolytic pathway such as hexokinase, PHO5 promoter, PGK promoter, GAP promoter, ADH promoter, gal 1 promoter, gal 10 promoter, heat shock protein promoter, MFα1 promoter and CUP 1 promoter.

Examples of suitable host cells are microorganisms belonging to the genera Saccharomyces, Schizosaccharomyces, Kluyveromyces, Trichosporon and Schwanniomyces, and specifically, Saccharomyces cerevisiae, Schizosaccharomyces pombe, Kluyveromyces lactis, Trichosporon pullulans and Schwanniomyces alluvius.

Introduction of the recombinant vector can be carried out by any of the methods for introducing DNA into yeast, for example, electroporation [Methods Enzymol., 194, 182 (1990)], the spheroplast method [Proc. Natl. Acad. Sci. USA, 84, 1929 (1978)], the lithium acetate method [J. Bacteriology, 153, 163 (1983)] and the method described in Proc. Natl. Acad. Sci. USA, 75, 1929 (1978).

When an animal cell is used as the host cell, pcDNAI, pcDM8 (commercially available from Funakoshi Co., Ltd.), pAGE107 [Japanese Published Unexamined Patent Application No. 22979/91; Cytotechnology, 3, 133 (1990)], pAS3-3 (Japanese Published Unexamined Patent Application No. 227075/90), pCDM8 [Nature, 329, 840 (1987)], pcDNAI/Amp (manufactured by Invitrogen Corp.), pREP4 (manufactured by Invitrogen Corp.), pAGE103 [J. Biochemistry, 101, 1307 (1987)], pAGE210, etc. can be used as the expression vector.

As the promoter, any promoters capable of expressing in animal cells can be used. Suitable promoters include the promoter of IE (immediate early) gene of cytomegalovirus (CMV), SV40 early promoter, the promoter of a retrovirus, metallothionein promoter, heat shock promoter, SRα promoter, etc. The enhancer of IE gene of human CMV may be used in combination with the promoter.

Examples of suitable host cells are human-derived Namalwa cells, monkey-derived COS cells, Chinese hamster-derived CHO cells, HBT5637 (Japanese Published Unexamined Patent Application No. 299/88), rat myeloma cells, mouse myeloma cells, cells derived from Syrian hamster kidney, embryonic stem cells and fertilized egg cells.

Introduction of the recombinant vector can be carried out by any of the methods for introducing DNA into animal cells, for example, electroporation [Cytotechnology, 3, 133 (1990)], the calcium phosphate method (Japanese Published Unexamined Patent Application No. 227075/90), lipofection [Proc. Natl. Acad. Sci. USA, 84, 7413 (1987)], the injection method (Manipulating the Mouse Embryo, A Laboratory Manual), the method using particle gun (gene gun) (Japanese Patent Nos. 2606856 and 2517813), the DEAE-dextran method [Biomanual Series 4—Methods of Gene Transfer, Expression and Analysis (Yodosha), edited by Takashi Yokota and Kenichi Arai (1994)] and the virus vector method (Manipulating the Mouse Embryo, Second Edition).

When an insect cell is used as the host cell, the protein can be expressed by the methods described in Current Protocols in Molecular Biology; Baculovirus Expression Vectors, A Laboratory Manual, W.H. Freeman and Company, New York (1992); Bio/Technology, 6, 47 (1988), etc.

That is, the recombinant vector and a baculovirus are cotransfected into insect cells to obtain a recombinant virus in the culture supernatant of the insect cells, and then insect cells are infected with the recombinant virus, whereby the protein can be expressed.

The gene transfer vectors useful in this method include pVL1392, pVL1393 and pBlueBacIII™ (products of Invitrogen Corp.).

An example of the baculovirus is Autographa californica nuclear polyhedrosis virus, which is a virus infecting insects belonging to the family Barathra.

Examples of the insect cells are Spodoptera frugiperda ovarian cells Sf9 and Sf21 [Current Protocols in Molecular Biology; Baculovirus Expression Vectors, A Laboratory Manual, W.H. Freeman and Company, New York (1992)] and Trichoplusia ni ovarian cell High 5 (manufactured by Invitrogen Corp.).

Cotransfection of the above recombinant vector and the above baculovirus into insect cells for the preparation of the recombinant virus can be carried out by the calcium phosphate method (Japanese Published Unexamined Patent Application No. 227075/90), lipofection [Proc. Natl. Acad. Sci. USA, 84, 7413 (1987)], etc.

When a plant cell is used as the host cell, Ti plasmid, tobacco mosaic virus vector, etc. can be used as the expression vector.

As the promoter, any promoters capable of expressing in plant cells can be used. Suitable promoters include 35S promoter of cauliflower mosaic virus (CaMV), rice actin 1 promoter, etc.

Examples of suitable host cells are cells of plants such as tobacco, potato, tomato, carrot, soybean, rape, alfalfa, rice, wheat and barley.

Introduction of the recombinant vector can be carried out by any of the methods for introducing DNA into plant cells, for example, the method using Agrobacterium (Japanese Published Unexamined Patent Application Nos. 140885/84 and 70080/85, WO94/00977), electroporation (Japanese Published Unexamined Patent Application No. 251887/85) and the method using particle gun (gene gun) (Japanese Patent Nos. 2606856 and 2517813).

Expression of the antibody gene can be carried out not only by direct expression but also by secretory production, expression of a fusion protein of the Fc region and another protein, etc. according to the methods described in Molecular Cloning, Second Edition, etc.

When the gene is expressed in yeast, an animal cell, an insect cell or a plant cell carrying an introduced gene relating to the synthesis of a sugar chain, an antibody molecule to which a sugar or a sugar chain is added by the introduced gene can be obtained.

The antibody composition can be produced by culturing the transformant obtained as above in a medium, allowing the antibody molecules to form and accumulate in the culture, and recovering them from the culture. Culturing of the transformant in a medium can be carried out by conventional methods for culturing the host cell.

For the culturing of the transformant obtained by using a eucaryote such as yeast as the host, any of natural media and synthetic media can be used insofar as it is a medium suitable for efficient culturing of the transformant which contains carbon sources, nitrogen sources, inorganic salts, etc. which can be assimilated by the host used.

As the carbon sources, any carbon sources that can be assimilated by the host can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

As the nitrogen sources, ammonia, ammonium salts of organic or inorganic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 40° C., and the culturing period is usually 16 hours to 7 days. The pH is maintained at 3.0 to 9.0 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with a recombinant vector comprising an inducible promoter is cultured, an inducer may be added to the medium, if necessary. For example, in the case of a microorganism transformed with a recombinant vector comprising lac promoter, isopropyl-β-D-thiogalactopyranoside or the like may be added to the medium; and in the case of a microorganism transformed with a recombinant vector comprising trp promoter, indoleacrylic acid or the like may be added.

For the culturing of the transformant obtained by using an animal cell as the host cell, generally employed media such as RPMI1640 medium [The Journal of the American Medical Association, 199, 519 (1967)], Eagle's MEM [Science, 122, 501 (1952)], Dulbecco's modified MEM [Virology, 8, 396 (1959)], 199 medium [Proceeding of the Society for the Biological Medicine, 73, 1 (1950)] and Whitten's medium [Developmental Engineering Experimentation Manual—Preparation of Transgenic Mice (Kodansha), edited by Motoya Katsuki (1987)], media prepared by adding fetal calf serum or the like to these media, etc. can be used as the medium.

Culturing is usually carried out under conditions of pH 6.0 to 8.0 at 30 to 40° C. for 1 to 7 days in the presence of 5% CO₂.

If necessary, antibiotics such as kanamycin and penicillin may be added to the medium during the culturing.

For the culturing of the transformant obtained by using an insect cell as the host cell, generally employed media such as TNM-FH Medium™ (manufactured by Pharmingen, Inc.), Sf-900 II SFM Medium™ (manufactured by Life Technologies, Inc.), ExCell 400™ and ExCell 405™ (manufactured by JRH Biosciences, Inc.) and Grace's Insect Medium [Nature, 195, 788 (1962)] can be used as the medium.

Culturing is usually carried out under conditions of pH 6.0 to 7.0 at 25 to 30° C. for 1 to 5 days.

If necessary, antibiotics such as gentamicin may be added to the medium during the culturing.

The transformant obtained by using a plant cell as the host cell may be cultured in the form of cells as such or after differentiation into plant cells or plant organs. For the culturing of such transformant, generally employed media such as Murashige-Skoog (MS) medium and White medium, media prepared by adding phytohormones such as auxin and cytokinin to these media, etc. can be used as the medium.

Culturing is usually carried out under conditions of pH 5.0 to 9.0 at 20 to 40° C. for 3 to 60 days.

If necessary, antibiotics such as kanamycin and hygromycin may be added to the medium during the culturing.

As described above, the antibody composition can be produced by culturing, according to a conventional culturing method, the transformant derived from an animal cell or a plant cell and carrying an expression vector into which DNA encoding the antibody molecule has been inserted, allowing the antibody composition to form and accumulate, and recovering the antibody composition from the culture.

Expression of the antibody gene can be carried out not only by direct expression but also by secretory production, fusion protein expression, etc. according to the methods described in Molecular Cloning, Second Edition.

The antibody composition may be produced by intracellular production by host cells, extracellular secretion by host cells or production on outer membranes by host cells. A desirable production method can be adopted by changing the kind of the host cells used or the structure of the antibody molecule to be produced.

When the antibody composition is produced in host cells or on outer membranes of host cells, it is possible to force the antibody composition to be secreted outside the host cells by applying the method of Paulson, et al. [J. Biol. Chem., 264, 17619 (1989)], the method of Lowe, et al. [Proc. Natl. Acad. Sci. USA, 86, 8227 (1989); Genes Develop., 4, 1288 (1990)], or the methods described in Japanese Published Unexamined Patent Application No. 336963/93, WO94/23021, etc.

That is, it is possible to force the desired antibody molecule to be secreted outside the host cells by inserting DNA encoding the antibody molecule and DNA encoding a signal peptide suitable for the expression of the antibody molecule into an expression vector, introducing the expression vector into the host cells, and then expressing the antibody molecule by use of recombinant DNA techniques.

It is also possible to increase the production of the antibody composition by utilizing a gene amplification system using a dihydrofolate reductase gene or the like according to the method described in Japanese Published Unexamined Patent Application No. 227075/90.

Further, the antibody composition can be produced using an animal having an introduced gene (non-human transgenic animal) or a plant having an introduced gene (transgenic plant) constructed by redifferentiation of animal or plant cells carrying the introduced gene.

When the transformant is an animal or plant, the antibody composition can be produced by raising or culturing the animal or plant in a usual manner, allowing the antibody composition to form and accumulate therein, and recovering the antibody composition from the animal or plant.

Production of the antibody composition using an animal can be carried out, for example, by producing the desired antibody composition in an animal constructed by introducing the gene according to known methods [American Journal of Clinical Nutrition, 63, 639S (1996); American Journal of Clinical Nutrition, 63, 627S (1996); Bio/Technology, 9, 830 (1991)].

In the case of an animal, the antibody composition can be produced, for example, by raising a non-human transgenic animal carrying the introduced DNA encoding the antibody molecule, allowing the antibody composition to form and accumulate in the animal, and recovering the antibody composition from the animal. The places where the antibody composition is formed and accumulated include milk (Japanese Published Unexamined Patent Application No. 309192/88), egg, etc. of the animal. As the promoter in this process, any promoters capable of expressing in an animal can be used. Preferred promoters include mammary gland cell-specific promoters such as α casein promoter, β casein promoter, β lactoglobulin promoter and whey acidic protein promoter.

Production of the antibody composition using a plant can be carried out, for example, by culturing a transgenic plant carrying the introduced DNA encoding the antibody molecule according to known methods [Soshiki Baiyo (Tissue Culture), 20 (1994); Soshiki Baiyo (Tissue Culture), 21 (1995); Trends in Biotechnology, 15, 45 (1997)], allowing the antibody composition to form and accumulate in the plant, and recovering the antibody composition from the plant.

When the antibody composition produced by the transformant carrying the introduced gene encoding the antibody molecule is expressed in a soluble form in cells, the cells are recovered by centrifugation after the completion of culturing and suspended in an aqueous buffer, followed by disruption using a sonicator, French press, Manton Gaulin homogenizer, Dynomill or the like to obtain a cell-free extract. A purified preparation of the antibody composition can be obtained by centrifuging the cell-free extract to obtain the supernatant and then subjecting the supernatant to ordinary means for isolating and purifying enzymes, e.g., extraction with a solvent, salting-out with ammonium sulfate, etc., desalting, precipitation with an organic solvent, anion exchange chromatography using resins such as diethylaminoethyl (DEAE)-Sepharose and DIAION HPA-75™ (manufactured by Mitsubishi Chemical Corporation), cation exchange chromatography using resins such as S-Sepharose FF™ (manufactured by Pharmacia), hydrophobic chromatography using resins such as butyl Sepharose and phenyl Sepharose, gel filtration using a molecular sieve, affinity chromatography, chromatofocusing, and electrophoresis such as isoelectric focusing, alone or in combination.

When the antibody composition is expressed as an inclusion body in cells, the cells are similarly recovered and disrupted, followed by centrifugation to recover the inclusion body of the antibody composition as a precipitate fraction. The recovered inclusion body of the antibody composition is solubilized with a protein-denaturing agent. The solubilized antibody solution is diluted or dialyzed, whereby the antibody composition is renatured to have normal conformation. Then, a purified preparation of the antibody composition can be obtained by the same isolation and purification steps as described above.

When the antibody composition is extracellularly secreted, the antibody composition or its derivative can be recovered in the culture supernatant. That is, the culture is treated by the same means as above, e.g., centrifugation, to obtain the culture supernatant. A purified preparation of the antibody composition can be obtained from the culture supernatant by using the same isolation and purification methods as described above.

As an example of the methods for obtaining the antibody composition of the present invention, the method for producing a humanized antibody composition is specifically described below. Other antibody compositions can also be obtained in a similar manner.

### (1) Construction of a Vector for Expression of Humanized Antibody

A vector for expression of humanized antibody is an expression vector for animal cells carrying inserted genes encoding CH and CL of a human antibody, which can be constructed by cloning each of the genes encoding CH and CL of a human antibody into an expression vector for animal cells.

The C regions of a human antibody may be CH and CL of any human antibody. Examples of the C regions include the C region of IgG1 subclass human antibody H chain (hereinafter referred to as hCγ1) and the C region of κ class human antibody L chain (hereinafter referred to as hCκ).

As the genes encoding CH and CL of a human antibody, a genomic DNA comprising exons and introns can be used. Also useful is a cDNA prepared by reverse transcription of an mRNA.

As the expression vector for animal cells, any vector for animal cells can be used so long as it is capable of inserting and expressing the gene encoding the C region of a human antibody. Suitable vectors include pAGE107 [Cytotechnology, 3, 133 (1990)], pAGE103 [J. Biochem., 101, 1307 (1987)], pHSG274 [Gene, 27, 223 (1984)], pKCR [Proc. Natl. Acad. Sci. USA, 78, 1527 (1981)] and pSG1βd2-4 [Cytotechnology, 4, 173 (1990)]. Examples of the promoter and enhancer for use in the expression vector for animal cells include SV40 early promoter and enhancer [J. Biochem., 101, 1307 (1987)], LTR of Moloney mouse leukemia virus [Biochem. Biophys. Res. Commun., 149, 960 (1987)] and immunoglobulin H chain promoter [Cell, 41, 479 (1985)] and enhancer [Cell, 33, 717 (1983)].

The vector for expression of humanized antibody may be either of the type in which the genes encoding antibody H chain and L chain exist on separate vectors or of the type in which both genes exist on the same vector (hereinafter referred to as tandem-type). The tandem-type ones are preferred in view of the easiness of construction of the vector for expression of humanized antibody, the easiness of introduction into animal cells, the balance between the expression of antibody H chain and that of antibody L chain in animal cells, etc. [J. Immunol. Methods, 167, 271 (1994)]. Examples of the tandem-type humanized antibody expression vectors include pKANTEX93 [Mol. Immunol., 37, 1035 (2000)] and pEE18 [Hybridoma, 17, 559 (1998)].

The constructed vector for expression of humanized antibody can be used for the expression of a human chimeric antibody and a human CDR-grafted antibody in animal cells.

(2) Obtaining of cDNA Encoding V Region of an Antibody Derived from a Non-Human Animal

cDNAs encoding VH and VL of an antibody derived from a non-human animal, e.g., a mouse antibody can be obtained in the following manner.

A cDNA is synthesized using, as a template, an mRNA extracted from a hybridoma cell producing a non-human animal-derived antibody which specifically binds to human CCR4. The synthesized cDNA is inserted into a vector such as a phage or a plasmid to prepare a cDNA library. A recombinant phage or recombinant plasmid carrying a cDNA encoding VH and a recombinant phage or recombinant plasmid carrying a cDNA encoding VL are isolated from the cDNA library using DNA encoding the C region or V region of a known mouse antibody as a probe. The entire nucleotide sequences of VH and VL of the desired mouse antibody on the recombinant phages or recombinant plasmids are determined, and the whole amino acid sequences of VH and VL are deduced from the nucleotide sequences.

Hybridoma cells producing a non-human animal-derived antibody which specifically binds to human CCR4 can be obtained by immunizing a non-human animal with human CCR4 represented by SEQ ID NO:43, preparing hybridomas from antibody-producing cells of the immunized animal and myeloma cells according to a known method (Antibodies: A Laboratory Manual, Cold Spring Harbor Laboratory, Chapter 14, 1998), selecting cloned hybridomas, culturing the selected hybridomas and purifying cells from the culture supernatant.

As the non-human animal, any animal can be used so long as hybridoma cells can be prepared from the animal. Suitable animals include mouse, rat, hamster and rabbit.

The methods for preparing total RNA from a hybridoma cell include the guanidine thiocyanate-cesium trifluoroacetate method [Methods in Enzymol., 154, 3 (1987)], and the methods for preparing mRNA from the total RNA include the oligo (dT) immobilized cellulose column method (Molecular Cloning: A Laboratory Manual, Cold Spring Harbor Lab. Press New York, 1989). Examples of the kits for preparing mRNA from a hybridoma cell include Fast Track mRNA Isolation Kit™ (manufactured by Invitrogen) and Quick Prep mRNA Purification Kit™ (manufactured by Pharmacia).

The methods for synthesizing the cDNA and preparing the cDNA library include conventional methods (Molecular Cloning: A Laboratory Manual, Cold Spring Harbor Lab. Press New York, 1989; Current Protocols in Molecular Biology, Supplement 1-34), or methods using commercially available kits such as SuperScript™ Plasmid System for cDNA Synthesis and Plasmid Cloning™ (manufactured by GIBCO BRL) and ZAP-cDNA Synthesis Kit™ (manufactured by STRATAGENE).

In preparing the cDNA library, the vector for inserting the cDNA synthesized using the mRNA extracted from a hybridoma cell as a template may be any vector so long as the cDNA can be inserted. Examples of suitable vectors include ZAP Express™ [Strategies, 5, 58 (1992)], pBluescript II SK(+)™ [Nucleic Acids Research, 17, 9494 (1989)], λZAP II™ (manufactured by STRATAGENE), λgt10, λgt11 [DNA Cloning: A Practical Approach, I, 49 (1985)], Lambda BlueMid™ (manufactured by Clontech), λExCell, pT7T3 18U (manufactured by Pharmacia), pcD2 [Mol. Cell. Biol., 3, 280 (1983)] and pUC18 [Gene, 33, 103 (1985)].

As Escherichia coli for introducing the cDNA library constructed with a phage or plasmid vector, any Escherichia coli can be used so long as the cDNA library can be introduced, expressed and maintained. Examples of suitable Escherichia coli include XL1-Blue MRF′ [Strategies, 5, 81 (1992)], C600 [Genetics, 39, 440 (1954)], Y1088, Y1090 [Science, 222, 778 (1983)], NM522 [J. Mol. Biol., 166, 1 (1983)], K802 [J. Mol. Biol., 16, 118 (1966)] and JM105 [Gene, 38, 275 (1985)].

The methods for selecting the cDNA clones encoding VH and VL of a non-human animal-derived antibody from the cDNA library include colony hybridization or plaque hybridization (Molecular Cloning: A Laboratory Manual, Cold Spring Harbor Lab. Press New York, 1989) using an isotope- or fluorescence-labeled probe. It is also possible to prepare the cDNAs encoding VH and VL by preparing primers and performing PCR (Molecular Cloning: A Laboratory Manual, Cold Spring Harbor Lab. Press New York, 1989; Current Protocols in Molecular Biology, Supplement 1-34) using the cDNA or cDNA library as a template.

The nucleotide sequences of the cDNAs selected by the above methods can be determined by cleaving the cDNAs with appropriate restriction enzymes, cloning the fragments into a plasmid such as pBluescript SK(−)™ (manufactured by STRATAGENE), and then analyzing the sequences by generally employed sequencing methods such as the dideoxy method of Sanger, et al. [Proc. Natl. Acad. Sci. USA, 74, 5463 (1977)] or by use of nucleotide sequencers such as ABI PRISM 377 DNA Sequencer (manufactured by Applied Biosystems).

The whole amino acid sequences of VH and VL are deduced from the determined nucleotide sequences and compared with the entire amino acid sequences of VH and VL of a known antibody (Sequences of Proteins of Immunological Interest, US Dept. Health and Human Services, 1991), whereby it can be confirmed that the obtained cDNAs encode amino acid sequences which completely comprise VH and VL of the antibody including secretory signal sequences.

Further, when the amino acid sequence of an antibody variable region or the nucleotide sequence of DNA encoding the variable region is already known, the DNA can be obtained by the following methods.

When the amino acid sequence is known, the desired DNA can be obtained by designing a DNA sequence encoding the variable region taking into consideration the frequency of occurrence of codons (Sequences of Proteins of Immunological Interest, US Dept. Health and Human Services, 1991), synthesizing several synthetic DNAs constituting approximately 100-nucleotides based on the designed DNA sequence, and carrying out PCR using the synthetic DNAs. When the nucleotide sequence is known, the desired DNA can be obtained by synthesizing several synthetic DNAs constituting approximately 100-nucleotides based on the nucleotide sequence information and carrying out PCR using the synthetic DNAs.

(3) Analysis of the Amino Acid Sequence of the V Region of an Antibody Derived from a Non-Human Animal

By comparing the whole amino acid sequences of VH and VL of the antibody including secretory signal sequences with the amino acid sequences of VH and VL of a known antibody (Sequences of Proteins of Immunological Interest, US Dept. Health and Human Services, 1991), it is possible to deduce the length of the secretory signal sequences and the N-terminal amino acid sequences and further to know the subgroup to which the antibody belongs. In addition, the amino acid sequences of CDRs of VH and VL can be deduced in a similar manner.

### (4) Construction of a Human Chimeric Antibody Expression Vector

A human chimeric antibody expression vector can be constructed by inserting the cDNAs encoding VH and VL of an antibody derived from a non-human animal into sites upstream of the genes encoding CH and CL of a human antibody in the vector for expression of humanized antibody described in the above 2 (1). For example, a human chimeric antibody expression vector can be constructed by ligating the cDNAs encoding VH and VL of an antibody derived from a non-human animal respectively to synthetic DNAs comprising the 3′-terminal nucleotide sequences of VH and VL of an antibody derived from a non-human animal and the 5′-terminal nucleotide sequences of CH and CL of a human antibody and also having recognition sequences for appropriate restriction enzymes at both ends, and inserting them into sites upstream of the genes encoding CH and CL of a human antibody in the vector for humanized antibody expression described in the above 2 (1) so as to express them in an appropriate form.

(5) Construction of cDNA Encoding V Region of a Human CDR-Grafted Antibody

cDNAs encoding VH and VL of a human CDR-grafted antibody can be constructed in the following manner. First, amino acid sequences of FRs of VH and VL of a human antibody for grafting CDRs of VH and VL of a non-human animal-derived antibody are selected. The amino acid sequences of FRs of VH and VL of a human antibody may be any of those derived from human antibodies. Suitable sequences include the amino acid sequences of FRs of VHs and VLs of human antibodies registered at databases such as Protein Data Bank, and the amino acid sequences common to subgroups of FRs of VHs and VLs of human antibodies (Sequences of Proteins of Immunological Interest, US Dept. Health and Human Services, 1991). In order to prepare a human CDR-grafted antibody having a sufficient activity, it is preferred to select amino acid sequences having as high a homology as possible (at least 60% or more) with the amino acid sequences of FRs of VH and VL of the non-human animal-derived antibody of interest.

Next, the amino acid sequences of CDRs of VH and VL of the non-human animal-derived antibody of interest are grafted to the selected amino acid sequences of FRs of VH and VL of a human antibody to design amino acid sequences of VH and VL of a human CDR-grafted antibody. The designed amino acid sequences are converted into DNA sequences taking into consideration the frequency of occurrence of codons in the nucleotide sequences of antibody genes (Sequences of Proteins of Immunological Interest, US Dept. Health and Human Services, 1991), and DNA sequences encoding the amino acid sequences of VH and VL of the human CDR-grafted antibody are designed. Several synthetic DNAs constituting approximately 100-nucleotides are synthesized based on the designed DNA sequences, and PCR is carried out using the synthetic DNAs. It is preferred to design 4 to 6 synthetic DNAs for each of the H chain and the L chain in view of the reaction efficiency of PCR and the lengths of DNAs that can be synthesized.

Cloning into the vector for humanized antibody expression constructed in the above 2 (1) can be easily carried out by introducing recognition sequences for appropriate restriction enzymes to the 5′ ends of synthetic DNAs present on both ends. After the PCR, the amplification products are cloned into a plasmid such as pBluescript SK(−)™ (manufactured by STRATAGENE) and the nucleotide sequences are determined by the method described in the above 2 (2) to obtain a plasmid carrying DNA sequences encoding the amino acid sequences of VH and VL of the desired human CDR-grafted antibody.

### (6) Modification of the Amino Acid Sequence of V Region of a Human CDR-Grafted Antibody

It is known that a human CDR-grafted antibody prepared merely by grafting CDRs of VH and VL of a non-human animal-derived antibody to FRs of VH and VL of a human antibody has a lower antigen-binding activity compared with the original non-human animal-derived antibody [BIO/TECHNOLOGY, 9, 266 (1991)]. This is probably because in VH and VL of the original non-human animal-derived antibody, not only CDRs but also some of the amino acid residues in FRs are involved directly or indirectly in the antigen-binding activity, and such amino acid residues are replaced by amino acid residues derived from FRs of VH and VL of the human antibody by CDR grafting. In order to solve this problem, attempts have been made in the preparation of a human CDR-grafted antibody to raise the lowered antigen-binding activity by identifying the amino acid residues in the amino acid sequences of FRs of VH and VL of a human antibody which are directly relating to the binding to an antigen or which are indirectly relating to it through interaction with amino acid residues in CDRs or maintenance of the tertiary structure of antibody, and modifying such amino acid residues to those derived from the original non-human animal-derived antibody [BIO/TECHNOLOGY, 9, 266 (1991)].

In the preparation of a human CDR-grafted antibody, it is most important to efficiently identify the amino acid residues in FR which are relating to the antigen-binding activity. For the efficient identification, construction and analyses of the tertiary structures of antibodies have been carried out by X ray crystallography [J. Mol. Biol., 112, 535 (1977)], computer modeling [Protein Engineering, 7, 1501 (1994)], etc. Although these studies on the tertiary structures of antibodies have provided much information useful for the preparation of human CDR-grafted antibodies, there is no established method for preparing a human CDR-grafted antibody that is adaptable to any type of antibody. That is, at present, it is still necessary to make trial-and-error approaches, e.g., preparation of several modifications for each antibody and examination of each modification for the relationship with the antigen-binding activity.

Modification of the amino acid residues in FRs of VH and VL of a human antibody can be achieved by PCR as described in the above 2 (5) using synthetic DNAs for modification. The nucleotide sequence of the PCR amplification product is determined by the method described in the above 2 (2) to confirm that the desired modification has been achieved.

### (7) Construction of a Human CDR-Grafted Antibody Expression Vector

A human CDR-grafted antibody expression vector can be constructed by inserting the cDNAs encoding VH and VL of the human CDR-grafted antibody constructed in the above 2 (5) and (6) into sites upstream of the genes encoding CH and CL of a human antibody in the vector for humanized antibody expression described in the above 2 (1). For example, a human CDR-grafted antibody expression vector can be constructed by introducing recognition sequences for appropriate restriction enzymes to the 5′ ends of synthetic DNAs present on both ends among the synthetic DNAs used for constructing VH and VL of the human CDR-grafted antibody in the above 2 (5) and (6), and inserting them into sites upstream of the genes encoding CH and CL of a human antibody in the vector for humanized antibody expression described in the above 2 (1) so as to express them in an appropriate form.

### (8) Stable Production of a Humanized Antibody

Transformants capable of stably producing a human chimeric antibody and a human CDR-grafted antibody (hereinafter collectively referred to as humanized antibody) can be obtained by introducing the humanized antibody expression vectors described in the above 2 (4) and (7) into appropriate animal cells.

Introduction of the humanized antibody expression vector into an animal cell can be carried out by electroporation [Japanese Published Unexamined Patent Application No. 257891/90; Cytotechnology, 3, 133 (1990)], etc.

As the animal cell for introducing the humanized antibody expression vector, any animal cell capable of producing a humanized antibody can be used.

Examples of the animal cells include mouse myeloma cell lines NS0 and SP2/0, Chinese hamster ovary cells CHO/dhfr− and CHO/DG44, rat myeloma cell lines YB2/0 and IR983F, Syrian hamster kidney-derived BHK cell, and human myeloma cell line Namalwa. Preferred are Chinese hamster ovary cell CHO/DG44 and rat myeloma cell line YB2/0.

After the introduction of the humanized antibody expression vector, the transformant capable of stably producing the humanized antibody can be selected using a medium for animal cell culture containing a compound such as G418 sulfate (hereinafter referred to as G418; manufactured by SIGMA) according to the method described in Japanese Published Unexamined Patent Application No. 257891/90. Examples of the media for animal cell culture include RPMI1640 medium (manufactured by Nissui Pharmaceutical Co., Ltd.), GIT Medium™ (manufactured by Nihon Pharmaceutical Co., Ltd.), EX-CELL 302 Medium™ (manufactured by JRH), IMDM medium (manufactured by GIBCO BRL), Hybridoma-SFM Medium™ (manufactured by GIBCO BRL), and media prepared by adding various additives such as fetal calf serum (hereinafter referred to as FCS) to these media. By culturing the obtained transformant in the medium, the humanized antibody can be formed and accumulated in the culture supernatant. The amount and the antigen-binding activity of the humanized antibody produced in the culture supernatant can be measured by enzyme-linked immunosorbent assay (hereinafter referred to as ELISA; Antibodies: A Laboratory Manual, Cold Spring Harbor Laboratory, Chapter 14, 1998; Monoclonal Antibodies: Principles and Practice, Academic Press Limited, 1996) or the like. The production of the humanized antibody by the transformant can be increased by utilizing a DHFR gene amplification system or the like according to the method described in Japanese Published Unexamined Patent Application No. 257891/90.

The humanized antibody can be purified from the culture supernatant of the transformant using a protein A column (Antibodies: A Laboratory Manual, Cold Spring Harbor Laboratory, Chapter 8, 1988; Monoclonal Antibodies: Principles and Practice, Academic Press Limited, 1996). In addition, purification methods generally employed for the purification of proteins can also be used. For example, the purification can be carried out by combinations of gel filtration, ion exchange chromatography, ultrafiltration and the like. The molecular weight of the H chain, L chain or whole antibody molecule of the purified humanized antibody can be measured by SDS-denatured polyacrylamide gel electrophoresis [hereinafter referred to as SDS-PAGE; Nature, 227, 680 (1970)], Western blotting (Antibodies: A Laboratory Manual, Cold Spring Harbor Laboratory, Chapter 12, 1988; Monoclonal Antibodies: Principles and Practice, Academic Press Limited, 1996), etc.

Shown above is the method for producing the antibody composition using an animal cell as the host. As described above, the antibody composition can also be produced using yeast, an insect cell, a plant cell, an animal or a plant by similar methods.

When a host cell inherently has the ability to express the antibody molecule, the antibody composition of the present invention can be produced by preparing a cell expressing the antibody molecule using the method described in the above 1, culturing the cell, and then purifying the desired antibody composition from the culture.

### 3. Evaluation of the Activity of the Antibody Composition

The protein amount, antigen-binding activity and effector function of the purified antibody composition can be measured using the known methods described in Monoclonal Antibodies, Antibody Engineering, etc.

Specifically, when the antibody composition is a humanized antibody, the activity to bind to an antigen or an antigenically positive cultured cell line can be measured by ELISA, the fluorescent antibody technique [Cancer Immunol. Immunother., 36, 373 (1993)], etc. The cytotoxic activity against an antigenically positive cultured cell line can be evaluated by measuring CDC activity, ADCC activity, etc. [Cancer Immunol. Immunother., 36, 373 (1993)].

The safety and therapeutic effect of the antibody composition in human can be evaluated using an appropriate animal model of a species relatively close to human, e.g., cynomolgus monkey.

### 4. Analysis of Sugar Chains in the Antibody Composition

The sugar chain structure of antibody molecules expressed in various cells can be analyzed according to general methods of analysis of the sugar chain structure of glycoproteins. For example, a sugar chain bound to an IgG molecule consists of neutral sugars such as galactose, mannose and fucose, amino sugars such as N-acetylglucosamine, and acidic sugars such as sialic acid, and can be analyzed by techniques such as sugar composition analysis and sugar chain structure analysis using two-dimensional sugar chain mapping.

### (1) Analysis of Neutral Sugar and Amino Sugar Compositions

The sugar chain composition of an antibody molecule can be analyzed by carrying out acid hydrolysis of sugar chains with trifluoroacetic acid or the like to release neutral sugars or amino sugars and analyzing the composition ratio.

Specifically, the analysis can be carried out by a method using a carbohydrate analysis system (BioLC™; product of Dionex). BioLC™ is a system for analyzing the sugar composition by HPAEC-PAD (high performance anion-exchange chromatography-pulsed amperometric detection) [J. Liq. Chromatogr., 6, 1577 (1983)].

The composition ratio can also be analyzed by the fluorescence labeling method using 2-aminopyridine. Specifically, the composition ratio can be calculated by fluorescence labeling an acid-hydrolyzed sample by 2-aminopyridylation according to a known method [Agric. Biol. Chem., 55(1), 283-284 (1991)] and then analyzing the composition by HPLC.

### (2) Analysis of Sugar Chain Structure

The sugar chain structure of an antibody molecule can be analyzed by two-dimensional sugar chain mapping [Anal. Biochem., 171, 73 (1988); Seibutsukagaku Jikkenho (Biochemical Experimentation Methods) 23—Totanpakushitsu Tosa Kenkyuho (Methods of Studies on Glycoprotein Sugar Chains), Gakkai Shuppan Center, edited by Reiko Takahashi (1989)]. The two-dimensional sugar chain mapping is a method of deducing a sugar chain structure, for example, by plotting the retention time or elution position of a sugar chain by reversed phase chromatography as the X axis and the retention time or elution position of the sugar chain by normal phase chromatography as the Y axis, and comparing them with the results on known sugar chains.

Specifically, a sugar chain is released from an antibody by hydrazinolysis of the antibody and subjected to fluorescence labeling with 2-aminopyridine (hereinafter referred to as PA) [J. Biochem., 95, 197 (1984)]. After being separated from an excess PA-treating reagent by gel filtration, the sugar chain is subjected to reversed phase chromatography. Then, each peak of the sugar chain is subjected to normal phase chromatography. The sugar chain structure can be deduced by plotting the obtained results on a two-dimensional sugar chain map and comparing them with the spots of a sugar chain standard (manufactured by Takara Shuzo Co., Ltd.) or those in the literature [Anal. Biochem., 171, 73 (1988)].

The structure deduced by the two-dimensional sugar chain mapping can be confirmed by carrying out mass spectrometry, e.g., MALDI-TOF-MS, of each sugar chain.

### 5. Immunoassay for Determining the Sugar Chain Structure of an Antibody Molecule

An antibody composition comprises an antibody molecule having different sugar chain structures binding to the Fc region of antibody. The antibody composition of the present invention, in which the ratio of a sugar chain in which fucose is not bound to the N-acetylglucosamine in the reducing end to the total complex type N-glycoside-linked sugar chains bound to the Fc region is 100%, has high ADCC activity. Such an antibody composition can be identified using the method for analyzing the sugar chain structure of an antibody molecule described in the above 4. Further, it can also be identified by immunoassays using lectins.

Discrimination of the sugar chain structure of an antibody molecule by immunoassays using lectins can be made according to the immunoassays such as Western staining, RIA (radioimmunoassay), VIA (viroimmunoassay), EIA (enzymoimmunoassay), FIA (fluoroimmunoassay) and MIA (metalloimmunoassay) described in the literature [Monoclonal Antibodies: Principles and Applications, Wiley-Liss, Inc. (1995); Enzyme Immunoassay, 3rd Ed., Igaku Shoin (1987); Enzyme Antibody Technique, Revised Edition, Gakusai Kikaku (1985); etc.], for example, in the following manner.

A lectin recognizing the sugar chain structure of an antibody molecule is labeled, and the labeled lectin is subjected to reaction with a sample antibody composition, followed by measurement of the amount of a complex of the labeled lectin with the antibody molecule.

Examples of lectins useful for determining the sugar chain structure of an antibody molecule include WGA (wheat-germ agglutinin derived from T. vulgaris), ConA (concanavalin A derived from C. ensiformis), RIC (toxin derived from R. communis), L-PHA (leukoagglutinin derived from P. vulgaris), LCA (lentil agglutinin derived from L. culinaris), PSA (pea lectin derived from P. sativum), AAL (Aleuria aurantia lectin), ACL (Amaranthus caudatus lectin), BPL (Bauhinia purpurea lectin), DSL (Datura stramonium lectin), DBA (Dolichos biflorus agglutinin), EBL (Elderberry balk lectin), ECL (Erythrina cristagalli lectin), EEL (Euonymus europaeus lectin), GNL (Galanthus nivalis lectin), GSL (Griffonia simplicifolia lectin), HPA (Helix pomatia agglutinin), HHL (Hippeastrum hybrid lectin), Jacalin, LTL (Lotus tetragonolobus lectin), LEL (Lycopersicon esculentum lectin), MAL (Maackia amurensis lectin), MPL (Maclura pomifera lectin), NPL (Narcissus pseudonarcissus lectin), PNA (peanut agglutinin), E-PHA (Phaseolus vulgaris erythroagglutinin), PTL (Psophocarpus tetragonolobus lectin), RCA (Ricinus communis agglutinin), STL (Solanum tuberosum lectin), SJA (Sophora japonica agglutinin), SBA (soybean agglutinin), UEA (Ulex europaeus agglutinin), VVL (Vicia villosa lectin) and WFA (Wisteria floribunda agglutinin).

It is preferred to use lectins specifically recognizing a sugar chain structure wherein fucose is bound to the N-acetylglucosamine in the reducing end in complex type N-glycoside-linked sugar chains. Examples of such lectins include lentil lectin LCA (lentil agglutinin derived from Lens culinaris), pea lectin PSA (pea lectin derived from Pisum sativum), broad bean lectin VFA (agglutinin derived from Vicia faba) and Aleuria aurantia lectin AAL (lectin derived from Aleuria aurantia).

### 6. Utilization of the Antibody Composition of the Present Invention

Since the antibody composition of the present invention specifically binds to human CCR4 and has high antibody-dependent cell-mediated cytotoxic activity, it is useful for the prevention and treatment of various diseases in which CCR4-expressing cells are concerned, including cancer and inflammatory diseases.

Examples of the cancer for which treatment by the antibody composition of the present invention is effective include blood cancers, particularly leukemia or lymphoma. Specific examples include adult T cell leukemia, ATL, mycosis fungoides, Sezary's syndrome, undifferentiated large cell lymphoma, non-specific T cell lymphoma and the like.

Examples of the inflammatory diseases for which treatment by the antibody composition of the present invention is effective include immunodiseases mediated by Th2 cells, such as acute or chronic bronchial hyperresponsiveness, bronchial asthma, atopic skin diseases including atopic dermatitis, chronic sinusitis, Churg-Strauss syndrome, nettle rash, pemphigus, eosinophilic myocarditis, allergic enterogastritis, allergic granulomatous angitis, allergic rhinitis and pollenosis.

In cancer such as malignant tumor, cancer cells proliferate abnormally. For example, in B cell lymphoma, specific B cells proliferate abnormally. Ordinary anti-tumor agents are characterized by the suppression of the growth of cancer cells. In contrast, antibodies having high antibody-dependent cell-mediated cytotoxic activity can treat cancer by injuring cancer cells which express antigens due to cell-killing effect, and therefore, they are more effective as therapeutic agents for cancer than ordinary anti-tumor agents. At present, therapeutic antibodies used as therapeutic agents for cancer have only insufficient anti-tumor effect when used alone and thus are used in combination with chemotherapy [Science, 280, 1197 (1998)]. If more potent anti-tumor effect is provided by the antibody composition of the present invention alone, the dependency on chemotherapy will be decreased and side effects will be reduced as well.

In allergic inflammatory diseases such as chronic airway hypersensitivity asthma, bronchial asthma, atopic skin diseases including atopic dermatitis, allergic rhinitis and pollinosis, inflammatory cells such as eosinophils and mast cells are induced to proliferate or differentiate by cytokines and chemokines such as IL-4, IL-5 and IL-13 produced by Th2 cells, and tissue injury or allergic reaction is induced via biofunctional molecules produced by these inflammatory cells. As therapeutic agents for the treatment of such immunodiseases mediated by Th2 cells, antagonists to cytokine such as IL-4, IL-5 or IL-13 produced by Th2 cells, cytokine production inhibitors thereof, and inhibitors against inflammatory cells and inflammatory biofunctional molecules have been developed. However, these therapeutic agents inhibit only a part of the complicated network among cytokines, chemokines and inflammatory cells and are not complete treatments. Since the antibody composition of the present invention specifically binds to the extracellular region of CCR4 and shows potent cytotoxic activity against CCR4-expressing cells, it can selectively eliminate Th2 cells which are CCR4-expressing cells present in the upstream part of allergic reactions and it further suppresses production of IL-4, IL-5, and IL-13 from Th2 cells. Accordingly, it is effective as a therapeutic agent for the above inflammatory diseases.

Since the antibody composition of the present invention includes no antibody molecule comprising sugar chains to which fucose is bound, its cytotoxic activity is enhanced. Therefore, patients of the above cancers, inflammatory diseases and the like who cannot be treated by an antibody composition which comprises an antibody molecule having sugar chains to which fucose is bound can be treated by the antibody composition of the present invention.

Especially, among the above diseases, since it is difficult to deliver a drug to the infiltration region of CCR4-expressing cells in the diseases such as cancers, bronchial asthma and chronic sinusitis, it is preferable that therapeutic effects can be obtained by a small amount of drug. Since the antibody composition of the present invention has high ADCC activity at a small amount, it is effective for treatment of these diseases.

A pharmaceutical composition comprising the antibody composition of the present invention may be administered alone as a therapeutic agent. However, it is preferably mixed with one or more pharmaceutically acceptable carriers and provided as a pharmaceutical preparation produced by an arbitrary method well known in the technical field of pharmaceutics.

It is desirable to administer the pharmaceutical composition by the route that is most effective for the treatment. Suitable administration routes include oral administration and parenteral administration such as intraoral administration, intratracheal administration, intrarectal administration, subcutaneous administration, intramuscular administration and intravenous administration. In the case of an antibody preparation, intravenous administration is preferable.

The pharmaceutical preparation may be in the form of spray, capsules, tablets, granules, syrup, emulsion, suppository, injection, ointment, tape, and the like.

The pharmaceutical preparations suitable for oral administration include emulsions, syrups, capsules, tablets, powders and granules.

Liquid preparations such as emulsions and syrups can be prepared using, as additives, water, sugars (e.g., sucrose, sorbitol and fructose), glycols (e.g., polyethylene glycol and propylene glycol), oils (e.g., sesame oil, olive oil and soybean oil), antiseptics (e.g., p-hydroxybenzoates), flavors (e.g., strawberry flavor and peppermint), and the like.

Capsules, tablets, powders, granules, etc. can be prepared using, as additives, excipients (e.g., lactose, glucose, sucrose and mannitol), disintegrators (e.g., starch and sodium alginate), lubricants (e.g., magnesium stearate and talc), binders (e.g., polyvinyl alcohol, hydroxypropyl cellulose and gelatin), surfactants (e.g., fatty acid esters), plasticizers (e.g., glycerin), and the like.

The pharmaceutical preparations suitable for parenteral administration include injections, suppositories and sprays.

Injections can be prepared using carriers comprising a salt solution, a glucose solution, or a mixture thereof, etc. It is also possible to prepare powder injections by freeze-drying the antibody composition according to a conventional method and adding sodium chloride thereto.

Suppositories can be prepared using carriers such as cacao butter, hydrogenated fat and carboxylic acid.

The antibody composition may be administered as such in the form of spray, but sprays may be prepared using carriers which do not stimulate the oral or airway mucous membrane of a recipient and which can disperse the antibody composition as fine particles to facilitate absorption thereof.

Suitable carriers include lactose and glycerin. It is also possible to prepare aerosols, dry powders, etc. according to the properties of the antibody composition and the carriers used. In preparing these parenteral preparations, the above-mentioned additives for the oral preparations may also be added.

The dose and administration frequency will vary depending on the desired therapeutic effect, the administration route, the period of treatment, the patient's age and body weight, etc. However, an appropriate dose of the active ingredient for an adult person is generally 10 μg/kg to 20 mg/kg per day.

The anti-tumor effect of the antibody composition against various tumor cells can be examined by in vitro tests such as CDC activity measurement and ADCC activity measurement and in vivo tests such as anti-tumor experiments using tumor systems in experimental animals (e.g., mice).

The CDC activity and ADCC activity measurements and anti-tumor experiments can be carried out according to the methods described in the literature [Cancer Immunology Immunotherapy, 36, 373 (1993); Cancer Research, 54, 1511 (1994); etc.].

Certain embodiments of the present invention are illustrated in the following examples. These examples are not to be construed as limiting the scope of the present invention.

### EXAMPLE 1

### Construction of CHO/DG44 Cell Line in which Both Alleles of α1,6-Fucosyltransferase (Hereinafter Referred to as FUT8) on the Genome have been Disrupted

The CHO/DG44 cell line comprising the deletion of a genome region for both alleles of FUT8 including the translation initiation codons was constructed according to the following steps.

1. Construction of Targeting Vector pKOFUT8Neo Comprising Exon 2 of Chinese Hamster FUT8 Gene

pKOFUT8Neo was constructed in the following manner using targeting vector pKOFUT8Puro comprising exon 2 of Chinese hamster FUT8 gene constructed by the method described in Example 13-1 of WO02/31140, and pKOSelectNeo™ (manufactured by Lexicon).

pKOSelectNeo™ (manufactured by Lexicon) was digested with the restriction enzyme AscI (manufactured by New England Biolabs) and subjected to agarose gel electrophoresis, and approximately 1.6 Kb AscI fragment comprising the neomycin resistance gene expression unit was recovered using GENECLEAN Spin Kit™ (manufactured by BIO 101).

After pKOFUT8Puro was digested with the restriction enzyme AscI (manufactured by New England Biolabs), the end of the DNA fragment with alkaline phosphatase derived from Escherichia coli C15 (manufactured by Takara Shuzo Co., Ltd.) was dephosphorylated. After the reaction, the DNA fragment was purified by phenol/chloroform extraction and ethanol precipitation.

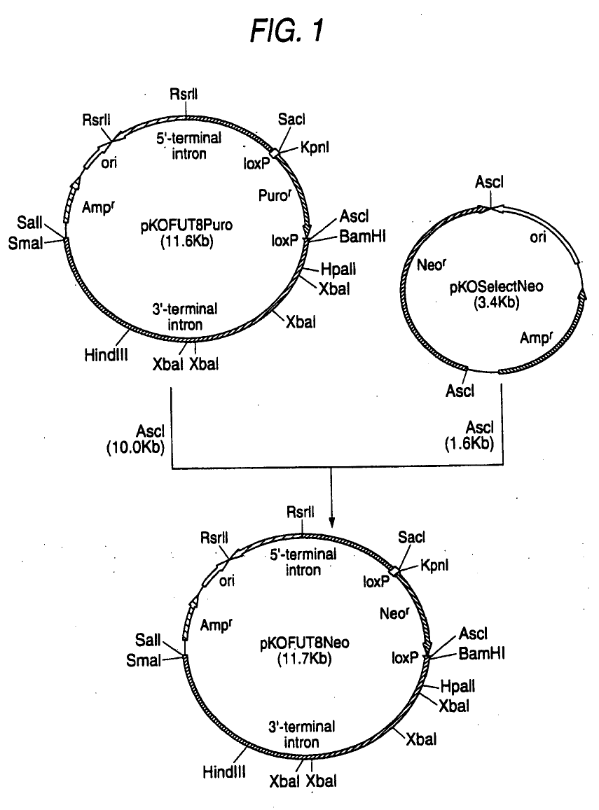

Sterilized water was added to 0.1 μg of the pKOSelectNeo-derived AscI fragment (approximately 1.6 Kb) and 0.1 μg of the pKOFUT8Puro-derived AscI fragment (approximately 10.1 Kb) obtained above to make up to 5 μl, and 5 μl of Ligation High (manufactured by Toyobo Co., Ltd.) was added thereto. The ligation reaction was carried out at 16° C. for 30 minutes. Escherichia coli DH5α was transformed using the resulting reaction mixture, and a plasmid DNA was prepared from each of the obtained ampicillin-resistant clones. The plasmid DNA was subjected to reaction using BigDye Terminator Cycle Sequencing Ready Reaction Kit v2.0™ (manufactured by Applied Biosystems) according to the attached instructions, and the nucleotide sequence was analyzed using DNA Sequencer ABI PRISM 377 (manufactured by Applied Biosystems). The thus obtained plasmid pKOFUT8Neo shown in FIG. 1 was used as a targeting vector for the subsequent preparation of FUT8 gene-hemi-knockout CHO cell line.

2. Preparation of Hemi-Knockout Cell Line in which One Copy of the FUT8 Gene on the Genome has been Disrupted
(1) Obtaining of a Cell Line in which the Targeting Vector pKOFUT8Neo has been Introduced

The Chinese hamster FUT8 genome region targeting vector pKOFUT8Neo constructed in Example 1-1 was introduced into Chinese hamster ovary-derived CHO/DG44 cells deficient in the dihydrofolate reductase gene (dhfr) [Somataic Cell and Molecular Genetics, 12, 555 (1986)] in the following manner.

pKOFUT8Neo was digested with the restriction enzyme SalI (manufactured by New England Biolabs) for linearization, and 4 μg of the linearized pKOFUT8Neo was introduced into 1.6×10⁶ CHO/DG44 cells by electroporation [Cytotechnology, 3, 133 (1990)]. The resulting cells were suspended in IMDM-dFBS (10)-HT(1) [IMDM medium (manufactured by Invitrogen) containing 10% dialysis FBS (manufactured by Invitrogen) and 1-fold concentration HT supplement (manufactured by Invitrogen)] and then seeded on a 10-cm dish for adherent cell culture (manufactured by Falcon). After culturing in a 5% CO₂ incubator at 37° C. for 24 hours, the medium was replaced with 10 ml of IMDM-dFBS(10) (IMDM medium containing 10% dialysis FBS) containing 600 μg/ml G418 (manufactured by Nacalai Tesque, Inc.). Culturing was carried out in a 5% CO₂ incubator at 37° C. for 15 days during which the above medium replacement was repeated every 3 to 4 days to obtain G418-resistant clones.

### (2) Confirmation of Homologous Recombination by Genomic PCR

Confirmation of the homologous recombination in the G418-resistant clones obtained in the above (1) was carried out by PCR using genomic DNA in the following manner.

The G418-resistant clones on a 96-well plate were subjected to trypsinization, and a 2-fold volume of a frozen medium (20% DMSO, 40% fetal calf serum and 40% IMDM) was added to each well to suspend the cells. One half of the cell suspension in each well was seeded on a flat-bottomed 96-well plate for adherent cells (manufactured by Asahi Techno Glass) to prepare a replica plate, while the other half was stored by cryopreservation as a master plate.

The neomycin-resistant clones on the replica plate were cultured using IMDM-dFBS(10) containing 600 μg/ml G418 in a 5% CO₂ incubator at 37° C. for one week, followed by recovery of cells. The genomic DNA of each clone was prepared from the recovered cells according to a known method [Analytical Biochemistry, 201, 331 (1992)] and then dissolved overnight in 30 μl of TE-RNase buffer (pH 8.0) (10 mmol/l Tris-HCL, 1 mmol/l EDTA, 200 μg/ml RNase A).

Primers used in the genomic PCR were designed as follows. Primers respectively having the sequences represented by SEQ ID NOs:39 and 40, which are contained in the sequence of the FUT8 genome region obtained by the method described in Example 12 of WO03/31140 (SEQ ID NO:13), were employed as forward primers. Primers respectively having the sequences represented by SEQ ID NOs:41 and 42 which specifically bind to the loxP sequence of the targeting vector were employed as reverse primers in the following polymerase chain reaction (PCR). A reaction mixture [25 μl; DNA polymerase ExTaq™ (manufactured by Takara Shuzo Co., Ltd.), ExTaq Buffer™ (manufactured by Takara Shuzo Co., Ltd.), 0.2 mmol/l dNTPs, 0.5 μmol/l each of the above primers (a combination of a forward primer and a reverse primer)] containing 10 μl of each genomic DNA solution prepared above was prepared, and PCR was carried out, after heating at 94° C. for 3 minutes, by cycles, one cycle consisting of reaction at 94° C. for one minute, reaction at 60° C. for one minute and reaction at 72° C. for 2 minutes.

After the PCR, the reaction mixture was subjected to 0.8% (w/v) agarose gel electrophoresis, and cell lines with which a specific amplification product (approximately 1.7 Kb) resulting from the homologous recombination was observed were judged to be positive clones.

### (3) Confirmation of Homologous Recombination by Genomic Southern Blotting

Confirmation of the homologous recombination in the positive clones obtained in the above (2) was carried out by Southern blotting using genomic DNA in the following manner.

From the master plates stored by cryopreservation in the above (2), a 96-well plate containing the positive clones found in (2) was selected. After the plate was allowed to stand in a 5% CO₂ incubator at 37° C. for 10 minutes, the cells in the wells corresponding to the positive clones were seeded on a flat-bottomed 24-well plate for adherent cells (manufactured by Greiner). After culturing using IMDM-dFBS(10) containing 600 μg/ml G418 in a 5% CO₂ incubator at 37° C. for one week, the cells were seeded on a flat-bottomed 6-well plate for adherent cells (manufactured by Greiner). The plate was subjected to culturing in a 5% CO₂ incubator at 37° C. and the cells were recovered. The genomic DNA of each clone was prepared from the recovered cells according to a known method [Nucleic Acids Research, 3, 2303 (1976)] and then dissolved overnight in 150 μl of TE-RNase buffer (pH 8.0).

The genomic DNA prepared above (12 μg) was digested with the restriction enzyme BamHI (manufactured by New England Biolabs), and a DNA fragment recovered by ethanol precipitation was dissolved in 20 μl of TE buffer (pH 8.0) (10 mmol/l Tris-HCL, 1 mmol/l EDTA) and then subjected to 0.6% (w/v) agarose gel electrophoresis. After the electrophoresis, the genomic DNA was transferred to a nylon membrane according to a known method [Proc. Natl. Acad. Sci. USA, 76, 3683 (1979)], followed by heat treatment of the nylon membrane at 80° C. for 2 hours for immobilization.

Separately, a probe used in the Southern blotting was prepared in the following manner. Primers respectively having the sequences represented by SEQ ID NOs:43 and 44, which are contained in the sequence of the FUT8 genome region obtained by the method described in Example 12 of WO03/31140 (SEQ ID NO:13), were prepared and used in the following PCR. A reaction mixture [20 μl; DNA polymerase ExTaq™ (manufactured by Takara Shuzo Co., Ltd.), ExTaq Buffer™ (manufactured by Takara Shuzo Co., Ltd.), 0.2 mmol/l dNTPs, 0.5 μmol/l each of the above primers] containing 4.0 ng of pFUT8fgE2-2 described in Example 12 of WO02/31140 as a template was prepared, and PCR was carried out, after heating at 94° C. for one minute, by 25 cycles, one cycle consisting of reaction at 94° C. for 30 seconds, reaction at 55° C. for 30 seconds and reaction at 74° C. for one minute.

After the PCR, the reaction mixture was subjected to 1.75% (w/v) agarose gel electrophoresis, and approximately 230 bp probe DNA fragment was recovered using GENECLEAN Spin Kit™ (manufactured by BIO101). A 5-μl portion of the obtained probe DNA solution was subjected to radiolabeling using [α-³²P] dCTP 1.75 MBq and Megaprime DNA Labelling system, dCTP™ (manufactured by Amersham Pharmacia Biotech).

Hybridization was carried out in the following manner. The above nylon membrane to which the genomic DNA digestion product had been transferred was put into a roller bottle and 15 ml of a hybridization solution [5×SSPE, 50×Denhardt's solution, 0.5% (w/v) SDS, 100 μg/ml salmon sperm DNA] was added thereto. Prehybridization was carried out at 65° C. for 3 hours. Then, the ³²P-labeled probe DNA was heat-denatured and put into the bottle, and hybridization was carried out at 65° C. overnight.

After the hybridization, the nylon membrane was immersed in 50 ml of a primary washing solution [2×SSC−0.1% (w/v) SDS] and washed by heating at 65° C. for 15 minutes. After this washing step was repeated twice, the nylon membrane was immersed in 50 ml of a secondary washing solution [0.2×SSC−0.1% (w/v) SDS] and washed by heating at 65° C. for 15 minutes. Then, the nylon membrane was exposed to an X-ray film at −80° C. for development.

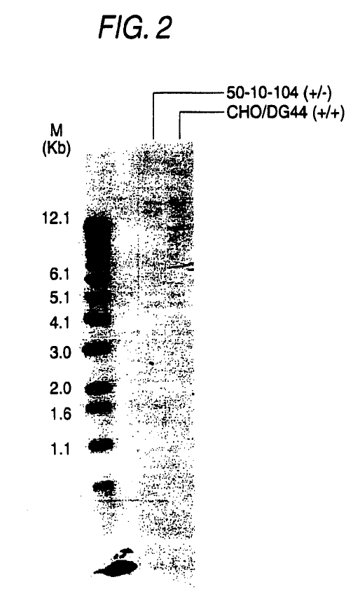

FIG. 2 shows the results of the analysis of the genomic DNAs of the parent cell line CHO/DG44 and the 50-10-104 cell line, which is the positive clone obtained in the above (2), according to the present method. In the CHO/DG44 cell line, only approximately 25.5 Kb fragment derived from the wild-type FUT8 allele was detected. On the other hand, in the positive clone, i.e. 50-10-104 cell line, approximately 20.0 Kb fragment peculiar to the allele which underwent homologous recombination was detected in addition to approximately 25.5 Kb fragment derived from the wild-type FUT8 allele. The quantitative ratio of these two kinds of fragments was 1:1, whereby it was confirmed that the 50-10-104 cell line was a hemi-knockout clone wherein one copy of the FUT8 allele was disrupted.

3. Preparation of CHO/DG44 Cell Line in which the FUT8 Gene on the Genome has been Double-Knocked Out
(1) Preparation of a Cell Line in which Targeting Vector pKOFUT8Puro has been Introduced

In order to disrupt the other FUT8 allele in the FUT8 gene-hemi-knockout clone obtained in the above 2, the Chinese hamster FUT8 gene exon 2 targeting vector pKOFUT8Puro described in Example 13-1 of WO02/31140 was introduced into the clone in the following manner.

pKOFUT8Puro was digested with the restriction enzyme SalI (manufactured by New England Biolabs) for linearization, and 4 μg of the linearized pKOFUT8Puro was introduced into 1.6×10⁶ cells of the FUT8 gene-hemi-knockout clone by electroporation [Cytotechnology, 3, 133 (1990)]. The resulting cells were suspended in IMDM-dFBS(10)-HT(1) and then seeded on a 10-cm dish for adherent cell culture (manufactured by Falcon). After culturing in a 5% CO₂ incubator at 37° C. for 24 hours, the medium was replaced with 10 ml of IMDM-dFBS(10)-HT(1) containing 15 μg/ml puromycin (manufactured by SIGMA). Culturing was carried out in a 5% CO₂ incubator at 37° C. for 15 days during which the above medium replacement was repeated every 7 days to obtain puromycin-resistant clones.

### (2) Confirmation of Homologous Recombination by Genomic Southern Blotting

Confirmation of the homologous recombination in the drug-resistant clones obtained in the above (1) was carried out by Southern blotting using genomic DNA in the following manner.

The puromycin-resistant clones were recovered into a flat-bottomed plate for adherent cells (manufactured by Asahi Techno Glass) according to a known method [Gene Targeting, Oxford University Press (1993)], followed by culturing using IMDM-dFBS(10)-HT(1) containing 15 μg/ml puromycin (manufactured by SIGMA) in a 5% CO₂ incubator at 37° C. for one week.

After the culturing, each clone on the above plate was subjected to trypsinization and the resulting cells were seeded on a flat-bottomed 24-well plate for adherent cells (manufactured by Greiner). After culturing using IMDM-dFBS(10)-HT(1) containing 15 μg/ml puromycin (manufactured by SIGMA) in a 5% CO₂ incubator at 37° C. for one week, the cells were subjected to trypsinization again and then seeded on a flat-bottomed 6-well plate for adherent cells (manufactured by Greiner). The plate was subjected to culturing in a 5% CO₂ incubator at 37° C. and the cells were recovered. The genomic DNA of each clone was prepared from the recovered cells according to a known method [Nucleic Acids Research, 3, 2303 (1976)] and then dissolved overnight in 150 μl of TE-RNase buffer (pH 8.0).

The genomic DNA prepared above (12 μg) was digested with the restriction enzyme BamHI (manufactured by New England Biolabs), and a DNA fragment recovered by ethanol precipitation was dissolved in 20 μl of TE buffer (pH 8.0) and then subjected to 0.6% (w/v) agarose gel electrophoresis. After the electrophoresis, the genomic DNA was transferred to a nylon membrane according to a known method [Proc. Natl. Acad. Sci. USA, 76, 3683 (1979)], followed by heat treatment of the nylon membrane at 80° C. for 2 hours for immobilization.

Separately, a probe used in the Southern blotting was prepared in the following manner. Primers respectively having the sequences represented by SEQ ID NOs:45 and 46, which specifically bind to the sequences closer to the 5′ end than the FUT8 genome region contained in the targeting vector, were prepared and used in the following PCR. A reaction mixture [20 μl; DNA polymerase ExTaq (manufactured by Takara Shuzo Co., Ltd.), ExTaq Buffer™ (manufactured by Takara Shuzo Co., Ltd.), 0.2 mmol/l dNTPs, 0.5 μmol/l each of the above primers] containing 4.0 ng of the plasmid pFUT8fgE2-2 described in Example 12 of WO02/31140 as a template was prepared, and PCR was carried out, after heating at 94° C. for one minute, by 25 cycles, one cycle consisting of reaction at 94° C. for 30 seconds, reaction at 55° C. for 30 seconds and reaction at 74° C. for one minute.

After the PCR, the reaction mixture was subjected to 1.75% (w/v) agarose gel electrophoresis, and approximately 230 bp probe DNA fragment was purified using GENECLEAN Spin Kit™ (manufactured by BIO101). A 5-μl portion of the obtained probe DNA solution was subjected to radiolabeling using [α-³²P] dCTP 1.75 MBq and Megaprime DNA Labelling System™, dCTP (manufactured by Amersham Pharmacia Biotech).

Hybridization was carried out in the following manner. The above nylon membrane to which the genomic DNA digestion product had been transferred was put into a roller bottle and 15 ml of a hybridization solution [5×SSPE, 50×Denhardt's solution, 0.5% (w/v) SDS, 100 μg/ml salmon sperm DNA] was added thereto. Prehybridization was carried out at 65° C. for 3 hours. Then, the ³²P-labeled probe DNA was heat-denatured and put into the bottle, and hybridization was carried out at 65° C. overnight.

After the hybridization, the nylon membrane was immersed in 50 ml of a primary washing solution [2×SSC−0.1% (w/v) SDS] and washed by heating at 65° C. for 15 minutes. After this washing step was repeated twice, the nylon membrane was immersed in 50 ml of a secondary washing solution [0.2×SSC−0.1% (w/v) SDS] and washed by heating at 65° C. for 15 minutes. Then, the nylon membrane was exposed to an X-ray film at −80° C. for development.

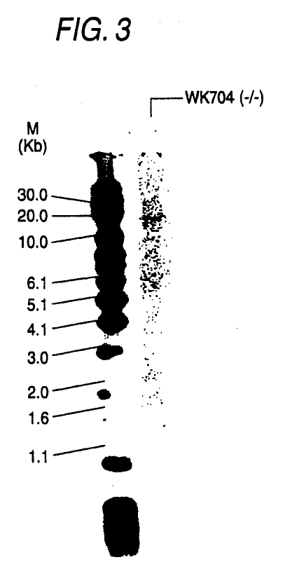

FIG. 3 shows the result of the analysis of the genomic DNA of the WK704 cell line, which is one of the puromycin-resistant clones obtained from the 50-10-104 cell line by the method described in the above (1), according to the present method. In the WK704 cell line, approximately 25.5 Kb fragment derived from the wild-type FUT8 allele was not detected and only approximately 20.0 Kb fragment specific to the allele which underwent homologous recombination (indicated by arrow in the figure) was detected. From this result, it was confirmed that the WK704 cell line was a clone wherein both FUT8 alleles were disrupted.

4. Removal of the Drug Resistance Genes from FUT8 Gene-Double-Knockout Cells

### (1) Introduction of Cre Recombinase Expression Vector

For the purpose of removing the drug resistance genes from the FUT8 gene-double-knockout clone obtained in the above item 3, the Cre recombinase expression vector pBS185™ (manufactured by Life Technologies) was introduced into the clone in the following manner.

pBS185 (4 μg) was introduced into 1.6×10⁶ cells of the FUT8 gene-double-knockout clone by electroporation [Cytotechnology, 3, 133 (1990)]. The resulting cells were suspended in 10 ml of IMDM-dFBS(10)-HT(1) and the suspension was diluted 20000-fold with the same medium. The diluted suspension was seeded on seven 10-cm dishes for adherent cell culture (manufactured by Falcon), followed by culturing in a 5% CO₂ incubator at 37° C. for 10 days to form colonies.

(2) Obtaining of a Cell Line in which the Cre Recombinase Expression Vector has been Introduced

Clones arbitrarily selected from the colonies obtained in the above (1) were recovered into a flat-bottomed plate for adherent cells (manufactured by Asahi Techno Glass) according to a known method [Gene Targeting, Oxford University Press (1993)], followed by culturing using IMDM-dFBS(10)-HT(1) in a 5% CO₂ incubator at 37° C. for one week.

After the culturing, each clone on the above plate was subjected to trypsinization, and a 2-fold volume of a frozen medium (20% DMSO, 40% fetal calf serum and 40% IMDM) was added to each well to suspend the cells. One half of the cell suspension in each well was seeded on a flat-bottomed 96-well plate for adherent cells (manufactured by Asahi Techno Glass) to prepare a replica plate, while the other half was stored by cryopreservation as a master plate.

The cells on the replica plate were cultured using IMDM-dFBS(10)-HT(1) containing 600 μg/ml G418 and 15 μg/ml puromycin in a 5% CO₂ incubator at 37° C. for one week. Positive clones in which the drug resistance genes inserted between loxP sequences has been removed by the expression of Cre recombinase have died in the presence of G418 and puromycin. The positive clones were selected in this manner.

### (3) Confirmation of Removal of the Drug Resistance Genes by Genomic Southern Blotting

Confirmation of the removal of the drug resistance genes in the positive clones selected in the above (2) was carried out by genomic Southern blotting in the following manner.

From the master plates stored by cryopreservation in the above (2), a 96-well plate containing the above positive clones was selected. After the plate was allowed to stand in a 5% CO₂ incubator at 37° C. for 10 minutes, the cells in the wells corresponding to the above clones were seeded on a flat-bottomed 24-well plate for adherent cells (manufactured by Greiner). After culturing using IMDM-dFBS(10)-HT(1) for one week, the cells were subjected to trypsinization and then seeded on a flat-bottomed 6-well plate for adherent cells (manufactured by Greiner). The plate was subjected to culturing in a 5% CO₂ incubator at 37° C. and the proliferated cells were recovered. The genomic DNA of each clone was prepared from the recovered cells according to a known method [Nucleic Acids Research, 3, 2303 (1976)] and then dissolved overnight in 150 μl of TE-RNase buffer (pH 8.0).

The genomic DNA prepared above (12 μg) was digested with the restriction enzyme NheI (manufactured by New England Biolabs), and a DNA fragment recovered by ethanol precipitation was dissolved in 20 μl of TE buffer (pH 8.0) and then subjected to 0.6% (w/v) agarose gel electrophoresis. After the electrophoresis, the genomic DNA was transferred to a nylon membrane according to a known method [Proc. Natl. Acad. Sci. USA, 76, 3683 (1979)], followed by heat treatment of the nylon membrane at 80° C. for 2 hours for immobilization.

Separately, a probe used in the Southern blotting was prepared in the following manner. PCR was carried out using primers respectively having the sequences represented by SEQ ID NOs:45 and 46, which specifically bind to the sequences closer to the 5′ end than the FUT8 genome region contained in the targeting vector. That is, a reaction mixture [20 μl; DNA polymerase ExTaq (manufactured by Takara Shuzo Co., Ltd.), ExTaq Buffer™ (manufactured by Takara Shuzo Co., Ltd.), 0.2 mmol/l dNTPs, 0.5 μmol/l each of the above primers] containing 4.0 ng of the plasmid pFUT8fgE2-2 described in Example 12 of WO02/31140 as a template was prepared, and PCR was carried out, after heating at 94° C. for one minute, by 25 cycles, one cycle consisting of reaction at 94° C. for 30 seconds, reaction at 55° C. for 30 seconds and reaction at 74° C. for one minute.

After the PCR, the reaction mixture was subjected to 1.75% (w/v) agarose gel electrophoresis, and approximately 230 bp probe DNA fragment was purified using GENECLEAN Spin Kit™ (manufactured by BIO101). A 5-μl portion of the obtained probe DNA solution was subjected to radiolabeling using [α-³²P] dCTP 1.75 MBq and Megaprime DNA Labelling System™, dCTP (manufactured by Amersham Pharmacia Biotech).

Hybridization was carried out in the following manner. The above nylon membrane to which the genomic DNA digestion product had been transferred was put into a roller bottle and 15 ml of a hybridization solution [5×SSPE, 50×Denhardt's solution, 0.5% (w/v) SDS, 100 μg/ml salmon sperm DNA] was added thereto. Prehybridization was carried out at 65° C. for 3 hours. Then, the ³²P-labeled probe DNA was heat-denatured and put into the bottle, and hybridization was carried out at 65° C. overnight.

After the hybridization, the nylon membrane was immersed in 50 ml of a primary washing solution [2×SSC−0.1% (w/v) SDS] and washed by heating at 65° C. for 15 minutes. After this washing step was repeated twice, the nylon membrane was immersed in 50 ml of a secondary washing solution [0.2×SSC−0.1% (w/v) SDS] and washed by heating at 65° C. for 15 minutes. Then, the nylon membrane was exposed to an X-ray film at −80° C. for development.

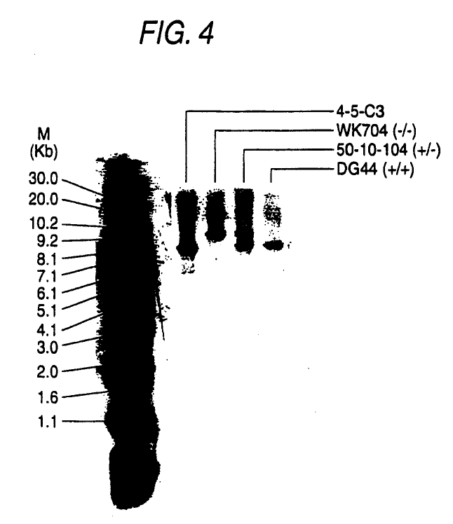

FIG. 4 shows the results of the analysis of the genomic DNAs of the parent cell line CHO/DG44, the 50-10-104 cell line described in the above item 2, the WK704 cell line described in the above item 3, and the 4-5-C3 cell line, which is one of the drug-sensitive clones obtained from the WK704 cell line by the method described in the above (2), according to the present method. In the CHO/DG44 cell line, only approximately 8.0 Kb DNA fragment derived from the wild-type FUT8 allele was detected. In the 50-10-104 cell line and the WK704 cell line, approximately 9.5 Kb DNA fragment derived from the allele which underwent homologous recombination was observed. On the other hand, in the 4-5-C3 cell line, only approximately 8.0 Kb DNA fragment resulting from the removal of the neomycin resistance gene (approximately 1.6 Kb) and the puromycin resistance gene (approximately 1.5 Kb) from the allele which underwent homologous recombination was detected. From the above results, it was confirmed that the drug resistance genes had been removed by Cre recombinase in the 4-5-C3 cell line.

Besides the 4-5-C3 cell line, plural FUT8 gene-double-knockout clones in which the drug-resistance gene had been removed (hereinafter referred to as FUT8 gene-double-knockout cells) were obtained.

### EXAMPLE 2

### Expression of an Anti-CCR4 Human CDR-Grafted Antibody Composition in FUT8 Gene-Double-Knockout Cell

### 1. Stable Expression in FUT8 Gene-Double-Knockout Cell

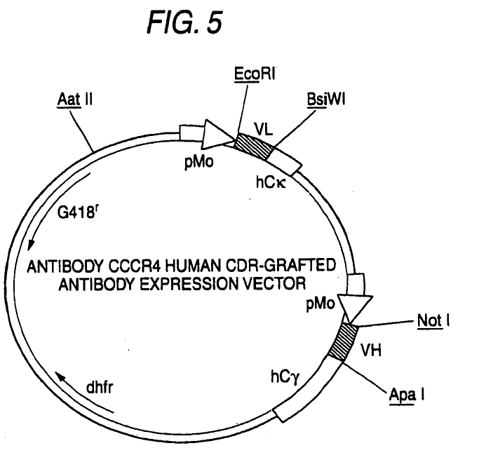

Cells stably producing an anti-CCR4 human CDR-grafted antibody were prepared by the method described in Example 1-2 (2) of WO02/31140 using the FUT8 gene-double-knockout cell described in Example 1-4 above and the parent cell CHO/DG44 as host cells. The anti-CCR4 human CDR-grafted antibody expression vector was prepared by the method described in Example 1 of WO03/18635. FIG. 5 shows the schematic view of the produced expression vector for the anti-CCR4 human CDR-grafted antibody.

As a result, transformants which are capable of growing in IMDM-dFBS(10) containing 500 μg/ml G418 and which produce the anti-CCR4 human CDR-grafted antibody were obtained. The transformants obtained from the parent cell CHO/DG44 and the FUT8 gene-double-knockout cell were designated DG44/CCR4 cell line and Ms705/CCR4 cell line, respectively.

### 2. Measurement of the Human IgG Antibody Concentration in Culture Supernatant (ELISA)

Goat anti-human IgG (manufactured by H & L) antibody (manufactured by American Qualex) was diluted with Phosphate Buffered Saline (hereinafter referred to as PBS) (manufactured by Invitrogen) to a concentration of 1 μg/ml and put into wells of a 96-well plate for ELISA (manufactured by Greiner) in an amount of 50 μl/well, followed by standing at 4° C. overnight for adsorption. After washing with PBS, PBS containing 1% BSA (hereinafter referred to as 1% BSA-PBS) (manufactured by Wako Pure Chemical Industries, Ltd.) was added to the wells in an amount of 100 μL/well, followed by reaction at room temperature for one hour to block the remaining active groups. Then, the 1% BSA-PBS was discarded, and 50 μl each of the culture supernatant of transformant or variously diluted solutions of an antibody purified from the culture supernatant were respectively added to the wells, followed by reaction at room temperature for one hour. After the reaction, the wells were washed with PBS containing 0.05% Tween 20 (hereinafter referred to as Tween-PBS) (manufactured by Wako Pure Chemical Industries, Ltd.). To each well was added 50 μl of peroxidase-labeled goat anti-human IgG (manufactured by H & L) antibody solution (manufactured by American Qualex) diluted 2000-fold with 1% BSA-PBS as a secondary antibody solution, followed by reaction at room temperature for one hour. After the reaction, the wells were washed with Tween-PBS, and 50 μl of ABTS substrate solution [a solution prepared by dissolving 0.55 g of 2,2′-azino-bis(3-ethylbenzothiazoline-6-sulfonic acid) ammonium (manufactured by Wako Pure Chemical Industries, Ltd.) in 1 liter of 0.1 M citrate buffer (pH 4.2) and adding thereto, just before use, 1 μl ml hydrogen peroxide (manufactured by Wako Pure Chemical Industries, Ltd.)] was added to each well to develop color. Then, the absorbance at 415 nm (hereinafter referred to as OD 415) was measured.

### 3. Purification of Anti-CCR4 Human CDR-Grafted Antibody Compositions

Anti-CCR4 human CDR-grafted antibody compositions produced by the transformants DG44/CHO and Ms705/CCR4 obtained in Example 2-1 were purified in the following manner.

Each transformant was suspended in IMDM-dFBS(10) containing 500 μg/ml G418 and 30 ml of the suspension was put into a 182-cm² flask (manufactured by Greiner), followed by culturing in a 5% CO₂ incubator at 37° C. for several days. When the cells became confluent, the culture supernatant was removed and the cells were washed with 25 ml of PBS, followed by addition of 30 ml of EXCELL301 Medium™ (manufactured by JRH Biosciences). After culturing in a 5% CO₂ incubator at 37° C. for 7 days, the cell suspension was recovered and subjected to centrifugation at 3000 rpm at 4° C. for 5 minutes to recover the supernatant. The supernatant was filtered through Millex GV Filter™ (pore size: 0.22 μm, manufactured by Millipore) for sterilization. The anti-CCR4 human CDR-grafted antibody composition was purified from the culture supernatant thus obtained using Mab Select™ (manufactured by Amersham Biosciences) column according to the attached instructions. The purified anti-CCR4 human CDR-grafted antibody compositions obtained from the DG44/CCR4 cell line and the Ms705/CCR4 cell line were designated DG44/CCR4 antibody and Ms705/CCR4 antibody, respectively. Also, the thus obtained Ms705/CCR4 cell line was deposited with International Patent Organism Depositary, National Institute of Advanced Industrial Science and Technology (Central 6, 1, Higashi 1-chome, Tsukuba-shi, Ibaraki, Japan) on Sep. 9, 2003 with accession No. FERM BP-8467.

### EXAMPLE 3

### Biological Activities of Anti-CCR4 Human CDR-Grafted Antibody Produced by FUT8 Gene-Double-Knockout Cell

### 1. Binding Activity of Anti-CCR4 Human CDR-Grafted Antibody to Human CCR4 Antigen (ELISA)

The binding activity of the DG44/CCR4 antibody and the Ms705/CCR4 antibody purified in Example 2-3 to human CCR4 antigen was measured in the following manner.

Compound 1 (SEQ ID NO:38) was selected as the peptide of the extracellular region of human CCR4 which can react with the anti-CCR4 human CDR-grafted antibody. In order to use Compound 1 in the activity measurement by ELISA, its conjugate with BSA (bovine serum albumin) (manufactured by Nacalai Tesque, Inc.) was prepared in the following manner and was used as the antigen. That is, 100 μl of 25 mg/ml SMCC [4-(N-maleimidomethyl)cyclohexane-1-carboxylic acid N-hydroxysuccinimide ester](manufactured by Sigma)-DMSO solution was added dropwise to 900 μl of PBS solution containing 10 mg of BSA under vortex, followed by gentle stirring for 30 minutes. After 1 ml of the reaction mixture was applied to a gel filtration column such as NAP-10 column equilibrated with 25 ml of PBS (manufactured by Invitrogen), the eluate eluted with 1.5 ml of PBS was used as a BSA-SMCC solution (BSA concentration was calculated by A₂₈₀ measurement). To 0.5 mg of Compound 1 were added 250 μl of PBS and then 250 μl of DMF to completely dissolve Compound 1, and then the above BSA-SMCC solution (BSA amount: 1.25 mg) was added under vortex, followed by gentle stirring for 3 hours. The reaction mixture was dialyzed against PBS at 4° C. overnight and sodium azide was added thereto to give a final concentration of 0.05%. The resulting mixture was filtered through a 0.22 μm filter to obtain a BSA-Compound 1 solution.

The BSA-Compound 1 solution thus prepared in the above was diluted with PBS to 1 μg/ml and put into wells of a 96-well plate for ELISA (manufactured by Greiner) in an amount of 50 μl/well, followed by standing at 4° C. overnight for adsorption. After washing with PBS, 1% BSA-PBS was added to the wells in an amount of 100 μl/well, followed by reaction at room temperature for one hour to block the remaining active groups. After the 1% BSA-PBS was discarded and each well was washed with Tween-PBS, 50 μl each of variously diluted solutions of the DG44/CCR4 antibody or the Ms705/CCR4 antibody prepared in Example 2-3 were respectively added to the wells, followed by reaction at room temperature for 2 hours. After the reaction, the wells were washed with Tween-PBS. To each well was added 50 μl of peroxidase-labeled goat anti-human IgG (H & L) antibody solution diluted 2000-fold with 1% BSA-PBS as a secondary antibody solution, followed by reaction at room temperature for one hour. After the reaction, the wells were washed with Tween-PBS, and 50 μl of ABTS substrate solution was added to each well to develop color, followed by measurement of OD415.

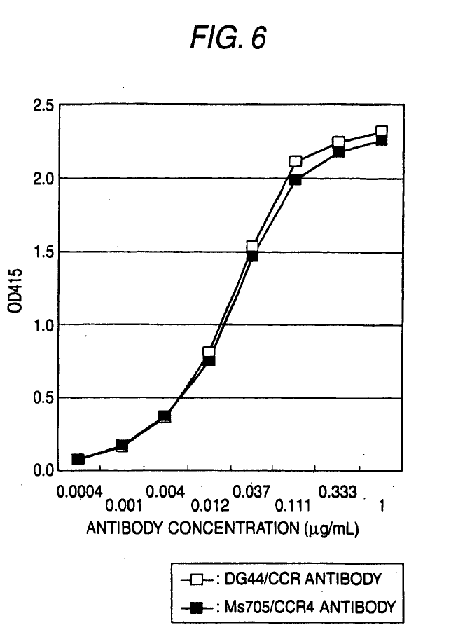

FIG. 6 shows the binding activity of the DG44/CCR4 antibody and the Ms705/CCR4 antibody to the CCR4 extracellular region peptide. Each antibody had an equal binding activity to the CCR4 extracellular region peptide.

### 2. In Vitro Cytotoxic Activity (ADCC Activity) of Anti-CCR4 Human CDR-Grafted Antibody Composition

The in vitro cytotoxic activity of the DG44/CCR4 antibody and the Ms705/CCR4 antibody obtained in Example 2-3 was measured in the following manner.

### (1) Preparation of a Target Cell Suspension

CCR4/EL4 cells which are mouse thymoma cell E4 cell line in which human CCR4 is highly expressed as described in WO01/64754 were washed with RPMI 1640-FCS(5) medium (RPMI 1640 medium (manufactured by GIBCO BRL) containing 5% FCS) by centrifugation and suspension and then adjusted to a density of 2×10⁵ cells/ml by using RPMI 1640-FCS(5) medium and used as the target cell suspension.

### (2) Preparation of an Effector Cell Suspension

Venous blood (50 ml) was collected from a healthy person and gently mixed with 0.5 ml of heparin sodium (manufactured by Shimizu Pharmaceutical Co., Ltd.). The monocyte layer was separated from this mixture using Lymphoprep™ (manufactured by AXIS SHIELD) according to the attached instructions. After being washed three times with RPMI1640-FCS(5) medium through centrifugation, the cells were suspended in the same medium at a density of 5×10⁶ cells/ml to give an effector cell suspension.

### (3) Measurement of ADCC Activity

The target cell suspension prepared in the above (1) (50 μl) was put into each well of a 96-well U-shaped bottom plate (manufactured by Falcon) (1×10⁴ cells/well). Then, 50 μl of the effector cell suspension prepared in (2) was added to each well (2.5×10⁵ cells/well; the ratio of effector cells to target cells becomes 25:1). Subsequently, each of the various anti-CCR4 human CDR-grafted antibodies was added to give a final concentration of 0.1 to 1000 ng/ml and to make a total volume of 150 μl, followed by reaction at 37° C. for 4 hours. After the reaction, the plate was subjected to centrifugation, and the lactate dehydrogenase (LDH) activity of the supernatant was measured by obtaining absorbance data using CytoTox96 Non-Radioactive Cytotoxicity Assay™ (manufactured by Promega) according to the attached instructions. The absorbance data for target cell spontaneous release were obtained by the same procedure as above using only the medium instead of the effector cell suspension and the antibody solution, and those for effector cell spontaneous release were obtained by the same procedure using only the medium instead of the target cell suspension and the antibody solution. The absorbance data for target cell total release were obtained by the same procedure as above using the medium instead of the antibody solution and the effector cell suspension, adding 15 μl of 9% Triton X-100 solution 45 minutes before the completion of the reaction, and measuring the LDH activity of the supernatant. The ADCC activity was calculated according to the following equation.

$$Cytotoxic activity ( \% ) = \frac{\begin{matrix}( \begin{matrix}Absorbance \\ of sample\end{matrix} ) - \\ ( \begin{matrix}Absorbance for \\ effector cell \\ spontaneous release \end{matrix} ) - ( \begin{matrix}Absorbance for \\ target cell \\ spontaneous release\end{matrix} )\end{matrix}}{( \begin{matrix}Absorbance for \\ target cell \\ total release\end{matrix} ) - ( \begin{matrix}Absorbance for \\ target cell \\ spontaneous release\end{matrix} )} \times 100$$

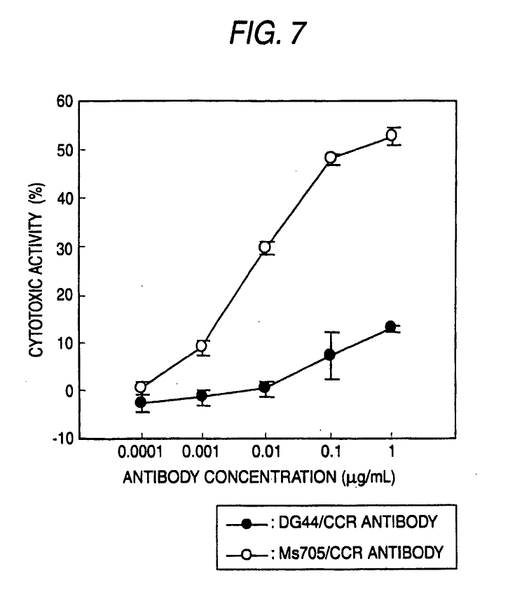

FIG. 7 shows the cytotoxic activities of the DG44/CCR4 antibody and the Ms705/CCR4 antibody against the CCR4/EL4 cells. The Ms705/CCR4 antibody showed higher ADCC activity than the DG44/CCR4 antibody at any antibody concentration.

### EXAMPLE 4

### Analysis of Monosaccharide Composition of Anti-CCR4 Human CDR-Grafted Antibody Composition Produced by FUT8 Gene-Double-Knockout Cell

Analysis of the neutral sugar and amino sugar composition of the DG44/CCR4 antibody and the Ms705/CCR4 antibody purified in Example 2-3 was carried out in the following manner.

After the antibody was dried under reduced pressure using a centrifugal concentrator, a 2.0 to 4.0 M trifluoroacetic acid solution was added thereto and acid hydrolysis was carried out at 100° C. for 2 to 4 hours to release neutral sugars and amino sugars from the protein. The trifluoroacetic acid solution was removed with a centrifugal concentrator, and the sugars were redissolved in deionized water and subjected to analysis using a carbohydrate analysis system (DX-500™ manufactured by Dionex). The analysis was carried out according to the elution program shown in Table 1 using CarboPac PA-1 Column™ and CarboPac PA-1 guard Column™ (manufactured by Dionex), a 10 to 20 mM solution of sodium hydroxide in deionized water as an eluting solution and a 500 mM solution of sodium hydroxide in deionized water as a washing solution.

TABLE 1
Elution program for neutral sugar and
amino sugar composition analysis

| Time (min.) | 0 | 35 | 35.1 | 45 | 45.1 | 58 |
|---|---|---|---|---|---|---|
| Eluting solution (%) | 100 | 100 | 0 | 0 | 100 | 100 |
| Washing solution (%) | 0 | 0 | 100 | 100 | 0 | 0 |

From the peak areas of neutral and amino sugar components in the obtained elution profile, the composition ratio of components (fucose, galactose and mannose) was calculated, regarding the value of N-acetylglucosamine as 4.

Table 2 shows the ratio of sugar chains having a structure in which fucose is not bound to the N-acetylglucosamine in the reducing end among the total complex type N-glycoside-linked sugar chains as calculated from the monosaccharide composition ratio of each antibody. In the DG44/CCR4 antibody, the ratio of sugar chains having a structure in which fucose is not bound was 8%. On the other hand, in the Ms705/CCR4 antibody, the peak of fucose was below the detection limit, whereby the ratio of sugar chains having a structure in which fucose is not bound was estimated to be close to 100%.

The above result indicates that fucose is not bound to the N-acetylglucosamine in the reducing end in complex type N-glycoside-linked sugar chains in the Ms705/CCR4 antibody.

TABLE 2
Ratio of sugar chains to which fucose is not bound in
anti-CCR4 human CDR-grafted antibody compositions

| | Ratio of sugar chains to which |
|---|---|
| Antibody | fucose is not bound |
| DG44/CCR4 antibody | 2% |
| Ms705/CCR4 antibody | 100% |

### EXAMPLE 5

### Analysis of Biological Activity of Anti-CCR4 Human CDR-Grafted Antibody Composition Having Sugar Chains to which Fucose is not Bound

In order to further reveal superiority of the anti-CCR4 human CDR-grafted antibody composition of the present invention, biological activity of an antibody composition having sugar chains to which fucose is not bound was compared with that of an antibody composition in which an antibody molecule having sugar chains to which fucose is not bound was mixed with an antibody molecule having sugar chains to which fucose is bound. Specifically, changes in the cytotoxic activity were examined in antibody compositions in which the Ms705/CCR4 antibody which is an anti-CCR4 human CDR-grafted antibody having sugar chains to which fucose is not bound was mixed with an anti-CCR4 human CDR-grafted antibody having sugar chains to which fucose is bound.

### (1) Preparation of Target Cell Suspension

The preparation was carried out according to the method described in Example 3-2(1).

### (2) Preparation of Effector Cell Suspension

A monocyte layer was separated according to the method described in Example 3-2(2) and the monocytes were suspended by using RPMI 1640-FCS(5) medium to a density of 4×10⁶ cells/ml to give the effector cell suspension.

### (3) Measurement of ADCC Activity

The target cell suspension prepared in the above (1) was dispensed at 50 μl into each well of a 96-well U bottom plate (manufactured by Falcon) (1×10⁴ cells/well). Next, the effector cell suspension prepared in the above (2) was added at 50 μl (2×10⁵ cells/well, the ratio of effector cells to target cells becomes 20:1). Subsequently, the Ms705/CCR4 antibody and the DG44/CCR4 antibody were added independently or as a mixture of both of them, adjusted to a total volume of 150 μl and then allowed to react at 37° C. for 4 hours. After the reaction, the plate was centrifuged, and lactate dehydrogenase (LDH) activity in the supernatant was measured using LDH-Cytotoxic Test Wako™ (manufactured by Wako Pure Chemical Industries) in accordance with the instructions attached thereto. The ADCC activity was calculated in accordance with the method described in Example 3-2.

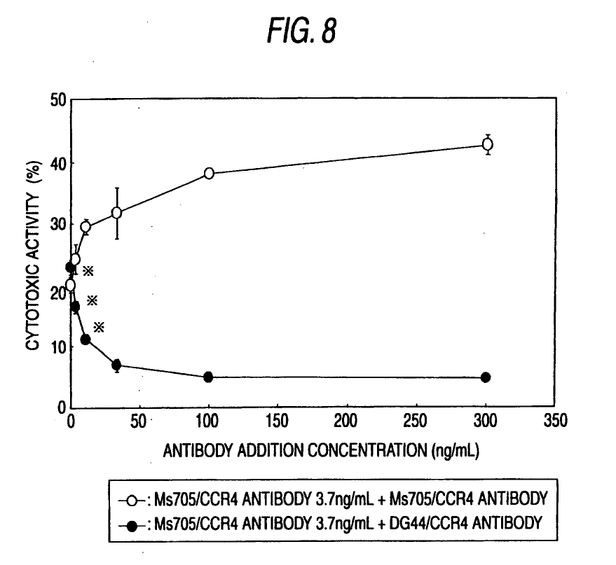

An anti-CCR4 human CDR-grafted antibody composition in which the ratio of an antibody having sugar chains to which fucose is not bound was changed was prepared by adding DG44/CCR4 antibody to a predetermined amount of the Ms705/CCR4 antibody, and its ADCC activity was measured. Specifically, an anti-CCR4 human CDR-grafted antibody composition in which 0 to 300 ng/ml of the DG44/CCR4 antibody was added to 3.7 ng/ml of Ms705/CCR4 antibody was prepared. The ADCC activity of the thus prepared antibody composition is shown in FIG. 8.

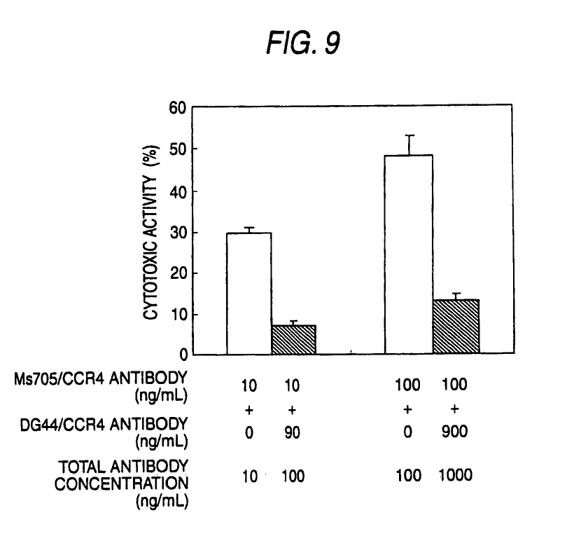

When the Ms705/CCR4 antibody was further added to 3.7 ng/ml of the Ms705/CCR4 antibody, increase of the ADCC activity was observed with increase of the total antibody concentration, but when the DG44/CCR4 antibody was further added to 3.7 ng/ml of the Ms705/CCR4 antibody, the ADCC activity of the thus prepared antibody composition was reduced on the contrary regardless of the increased total antibody concentration. This result showed that an antibody molecule having a sugar chain to which fucose is bound inhibits ADCC activity of an antibody molecule having a sugar chain to which fucose is not bound. Also, in the case of antibody compositions in which an antibody molecule having sugar chains to which fucose is bound was mixed with an antibody molecule having sugar chains to which fucose is not bound, an antibody composition in which the ratio of the antibody molecule having sugar chains to which fucose is not bound was 20% or more showed markedly higher ADCC activity than an antibody composition in which said ratio was less than 20%. ADCC activities of an antibody sample of 10 ng/ml of the Ms705/CCR4 antibody and an antibody sample prepared by mixing 10 ng/ml of the Ms705/CCR4 antibody with a 9-fold amount, namely 90 ng/ml, of the DG44/CCR4 antibody are shown as a graph in FIG. 9. The ADCC activity of the Ms705/CCR4 antibody was sharply reduced by the addition of DG44/CCR4 antibody. Even when antibody concentration of the antibody composition was increased to 100 times or more while keeping the existing ratio of the Ms705/CCR4 antibody and the DG44/CCR4 antibody at 1/9, the ADCC activity was still fell short of that of the 10 ng/ml Ms705/CCR4 antibody sample. Based on the above, it was found that an antibody molecule having sugar chains to which fucose is bound inhibits the ADCC activity of an antibody molecule having sugar chains to which fucose is not bound, and that the conventional antibody compositions cannot exert its ADCC activity similar to that of the antibody composition of the present invention.

Accordingly, patients who were unable to be treated by the conventional antibody compositions can be treated by the antibody composition of the present invention. Since the antibody composition of the present invention has high activity of injuring CCR4-expressing cells, it is clinically useful as a pharmaceutical composition for the treatment of CCR4-related diseases such as cancer and inflammatory diseases.

While the invention has been described in detail and with reference to specific embodiments thereof, it will be apparent to one skill in the art that various changes and modifications can be made therein without departing from the spirit and scope thereof. All references cited herein are incorporated in their entirety.

This application is based on Japanese application No. 2003-350162 filed on Oct. 8, 2003 and U.S. provisional patent application No. 60/572,784 filed on May 21, 2004, the entire contents of which are incorporated hereinto by reference.

1-53. (canceled)

54. A method for treating diseases related to a human CC chemokine receptor 4 (CCR4), which comprises administering a composition comprising an antibody to a subject in need of such treatment, the antibody being a recombinant antibody molecule which specifically binds to an extracellular region of the CCR4 and has complex type N-glycoside-linked sugar chains in the Fc region, wherein the complex type N-glycoside-linked sugar chains have a structure in which fucose is not bound to 6-position of N-acetylglucosamine in the reducing end through an α-bond in the sugar chains, wherein 100% of the recombinant antibody molecules in the composition do not possess N-glycoside-linked sugar chains in which fucose is bound to 6-position of N-acetylglucosamine in the reducing end through an α-bond, and the disease related to a human CCR4 being selected from the group consisting of blood cancers and Th2 cells-associated inflammatory diseases.

55-56. (canceled)

57. The method according to claim 54, wherein the extracellular region is an extracellular region selected from the group consisting of the sequences at positions 1 to 39, positions 98 to 112, positions 176 to 206 and positions 271 to 284 of the amino acid sequence represented by SEQ ID NO:36.

58. The method according to claim 54, wherein the extracellular region is an epitope existing at positions 2 to 29 of the amino acid sequence represented by SEQ ID NO:36.

59. The method according to claim 54, wherein the extracellular region is an epitope existing at positions 13 to 29 of the amino acid sequence represented by SEQ ID NO:36.

60. The method according to claim 54, wherein the extracellular region is an epitope existing at positions 13 to 25 of the amino acid sequence represented by SEQ ID NO:36.

61. The method according to claim 60, wherein the antibody has lower binding activity to a peptide comprising the sequence at positions 13 to 25 of the amino acid sequence represented by SEQ ID NO:36 wherein at least one of tyrosine residues at positions 16, 19, 20 and 22 is sulfated, in comparison with its binding activity to a peptide comprising the sequence at positions 13 to 25 of the amino acid sequence represented by SEQ ID NO:36.

62. The method according to claim 54, wherein the antibody has no reactivity to a human blood platelet.

63. The method according to claim 54, wherein the antibody has no activity of inhibiting a binding of a CCR4 ligand to the CCR4, the CCR4 ligand being TARC (thymus and activation-regulated chemokine) or MDC (macrophage-derived chemokine).

64. The method according to claim 54, wherein the antibody has cytotoxic activity against a CCR4-expressing cell.

65. The method according to claim 64, wherein the antibody has higher cytotoxic activity against a CCR4-expressing cell than a monoclonal antibody produced by a non-human animal-derived hybridoma.

66. The method according to claim 64, wherein the CCR4-expressing cell is a helper T cell.

67. The method according to claim 64, wherein the cytotoxic activity is antibody-dependent cell-mediated cytotoxic (ADCC) activity.

68. The method according to claim 54, wherein the antibody comprises complementarity determining region (CDR) 1, CDR 2 and CDR 3 of an antibody molecule heavy chain (H chain) variable region (V region) consisting of the amino acid sequences represented by SEQ ID NOs:14, 15 and 16, respectively; and CDR 1, CDR 2 and CDR 3 of an antibody molecule light chain (L chain) V region consisting of the amino acid sequences represented by SEQ ID NOs:17, 18 and 19, respectively.

69. The method according to claim 54, wherein the antibody is a human chimeric antibody, a human CDR-grafted antibody or a human antibody.

70. The method according to claim 69, wherein the antibody comprises CDRs of heavy chain (H chain) variable region (V region) and light chain (L chain) V region of a monoclonal antibody which specifically binds to the CCR4.

71. The method according to claim 70, wherein the heavy chain (H chain) variable region (V region) of the human chimeric antibody comprises the amino acid sequence represented by SEQ ID NO:21.

72. The method according to claim 70, wherein the light chain (L chain) variable region (V region) of the human chimeric antibody comprises the amino acid sequence represented by SEQ ID NO:23.

73. The method according to claim 70, wherein the heavy chain (H chain) variable region (V region) of the antibody molecule comprises the amino acid sequence represented by SEQ ID NO:21 and the light chain (L chain) V region of the antibody molecule comprises the amino acid sequence represented by SEQ ID NO:23.

74. The method according to claim 69, wherein the human CDR-grafted antibody comprises CDRs of heavy chain (H chain) variable region (V region) and light chain (L chain) V region of a monoclonal antibody which specifically binds to the CCR4, and framework regions (FRs) of H chain V region and L chain V region of a human antibody.

75. The method according to claim 69, wherein the human CDR-grafted antibody comprises CDRs of heavy chain (H chain) variable region (V region) and light chain (L chain) V region of a monoclonal antibody which specifically binds to the CCR4, FRs of H chain V region and L chain V region of a human antibody, and H chain constant region (C region) and L chain C region of a human antibody.

76. The method according to claim 70, wherein the heavy chain (H chain) variable region (V region) of the human CDR-grafted antibody comprises the amino acid sequence represented by SEQ ID NO:24 or an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24.

77. The method according to claim 70, wherein the heavy chain (H chain) variable region (V region) of the human CDR-grafted antibody comprises the amino acid sequence represented by SEQ ID NO:25 or an amino acid sequence in which at least one amino acid residue selected from the group consisting of Thr at position 28 and Ala at position 97 is substituted with another amino acid residue in the amino acid sequence represented by SEQ ID NO:25.

78. The method according to claim 70, wherein the light chain (L chain) variable region (V region) of the human CDR-grafted antibody comprises the amino acid sequence represented by SEQ ID NO:26 or an amino acid sequence in which at least one amino acid residue selected from the group consisting of Ile at position 2, Val at position 3, GIn at position 50 and Val at position 88 is substituted with another amino acid residue in the amino acid sequence represented by SEQ ID NO:26.

79. The method according to claim 70, wherein the heavy chain (H chain) variable region (V region) of the human CDR-grafted antibody comprises the amino acid sequence represented by SEQ ID NO:24 or an amino acid sequence in which at least one amino acid residue selected from the group consisting of Ala at position 40, Gly at position 42, Lys at position 43, Gly at position 44, Lys at position 76 and Ala at position 97 is substituted with another amino acid residue in the amino acid sequence represented by SEQ ID NO:24; and the light chain (L chain) V region of the antibody molecule comprises the amino acid sequence represented by SEQ ID NO:26 or an amino acid sequence in which at least one amino acid residue selected from the group consisting of Ile at position 2, Val at position 3, GIn at position 50 and Val at position 88 is substituted with another amino acid residue in the amino acid sequence represented by SEQ ID NO:26.

80. The method according to claim 70, wherein the heavy chain (H chain) variable region (V region) of the human CDR-grafted antibody comprises the amino acid sequence represented by SEQ ID NO:25 or an amino acid sequence in which at least one amino acid residue selected from the group consisting of Thr at position 28 and Ala at position 97 is substituted with another amino acid residue in the amino acid sequence represented by SEQ ID NO:25; and the light chain (L chain) V region of the antibody molecule comprises the amino acid sequence represented by SEQ ID NO:26 or an amino acid sequence in which at least one amino acid residue selected from the group consisting of Ile at position 2, Val at position 3, GIn at position 50 and Val at position 88 is substituted with another amino acid residue in the amino acid sequence represented by SEQ ID NO:26.

81. The method according to claim 70, wherein the heavy chain (H chain) variable region (V region) of the human CDR-grafted antibody comprises an amino acid sequence selected from the group consisting of the amino acid sequences represented by SEQ ID NOs:24, 25, 27, 28, 29, 30, 31 and 32.

82. The method according to claim 70, wherein the light (L chain) variable region (V region) of the human CDR-grafted antibody comprises an amino acid sequence selected from the group consisting of the amino acid sequences represented by SEQ ID NOs:26, 33, 34 and 35.

83. The method according to claim 70, wherein the heavy chain (H chain) variable region (V region) of the human CDR-grafted antibody comprises an amino acid sequence selected from the group consisting of the amino acid sequences represented by SEQ ID NOs:24, 25, 27, 28, 29, 30, 31 and 32; and the light chain (L chain) V region of the antibody molecule comprises an amino acid sequence selected from the group consisting of the amino acid sequences represented by SEQ ID NOs:33, 34 and 35.

84. The method according to claim 70, wherein the heavy chain (H chain) variable region (V region) of the human CDR-grafted antibody comprises the amino acid sequence represented by SEQ ID NO:27 or 28, and the light chain (L chain) V region of the antibody molecule comprises the amino acid sequence represented by SEQ ID NO:35.


SEQUENCE LISTING (46 sequences)

SEQ ID NO: 1 - DNA, 1504 bp, Cricetulus griseus
  CDS (1)..(1119)
atggctcacgctcccgctagctgcccgagctccaggaactctggggacggcgataagggcaagcccaggaaggtggcgctcatcacgggcatcaccggccaggatggctcatacttggcagaattcctgctggagaaaggatacgaggttcatggaattgtacggcgatccagttcatttaatacaggtcgaattgaacatttatataagaatccacaggctcatattgaaggaaacatgaagttgcactatggtgacctcaccgacagcacctgcctagtaaaaatcatcaatgaagtcaaacctacagagatctacaatcttggtgcccagagccatgtcaagatttcctttgacttagcagagtacactgcagatgttgatggagttggcaccttgcggcttctggatgcaattaagacttgtggccttataaattctgtgaagttctaccaggcctcaactagtgaactgtatggaaaagtgcaagaaataccccagaaagagaccacccctttctatccaaggtcgccctatggagcagccaaactttatgcctattggattgtagtgaactttcgagaggcttataatctctttgcggtgaacggcattctcttcaatcatgagagtcctagaagaggagctaattttgttactcgaaaaattagccggtcagtagctaagatttaccttggacaactggaatgtttcagtttgggaaatctggacgccaaacgagactggggccatgccaaggactatgtcgaggctatgtggctgatgttacaaaatgatgaaccagaggactttgtcatagctactggggaagttcatagtgtccgtgaatttgttgagaaatcattcatgcacattggaaagaccattgtgtgggaaggaaagaatgaaaatgaagtgggcagatgtaaagagaccggcaaaattcatgtgactgtggatctgaaatactaccgaccaactgaagtggacttcctgcagggagactgctccaaggcgcagcagaaactgaactggaagccccgcgttgcctttgacgagctggtgagggagatggtgcaagccgatgtggagctcatgagaaccaaccccaacgcctgagcacctctacaaaaaaattcgcgagacatggactatggtgcagagccagccaaccagagtccagccactcctgagaccatcgaccataaaccctcgactgcctgtgtcgtccccacagctaagagctgggccacaggtttgtgggcaccaggacggggacactccagagctaaggccacttcgcttttgtcaaaggctcctctcaatgattttgggaaatcaagaagtttaaaatcacatactcattttacttgaaattatgtcactagacaacttaaatttttgagtcttgagattgtttttctcttttcttattaaatgatctttctatgacccagcaaaaaaaaaaaaaaaaagggatataaaaaaaaaaaaaaaaaaaaaa

SEQ ID NO: 2 - protein, 372 aa, Cricetulus griseus
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 3 - DNA, 1316 bp, Cricetulus griseus
gccccgccccctccacctggaccgagagtagctggagaattgtgcaccggaagtagctcttggactggtggaaccctgcgcaggtgcagcaacaatgggtgagccccagggatccaggaggatcctagtgacagggggctctggactggtgggcagagctatccagaaggtggtcgcagatggcgctggcttacccggagaggaatgggtgtttgtctcctccaaagatgcagatctgacggatgcagcacaaacccaagccctgttccagaaggtacagcccacccatgtcatccatcttgctgcaatggtaggaggccttttccggaatatcaaatacaacttggatttctggaggaagaatgtgcacatcaatgacaacgtcctgcactcagctttcgaggtgggcactcgcaaggtggtctcctgcctgtccacctgtatcttccctgacaagaccacctatcctattgatgaaacaatgatccacaatggtccaccccacagcagcaattttgggtactcgtatgccaagaggatgattgacgtgcagaacagggcctacttccagcagcatggctgcaccttcactgctgtcatccctaccaatgtctttggacctcatgacaacttcaacattgaagatggccatgtgctgcctggcctcatccataaggtgcatctggccaagagtaatggttcagccttgactgtttggggtacagggaaaccacggaggcagttcatctactcactggacctagcccggctcttcatctgggtcctgcgggagtacaatgaagttgagcccatcatcctctcagtgggcgaggaagatgaagtctccattaaggaggcagctgaggctgtagtggaggccatggacttctgtggggaagtcacttttgattcaacaaagtcagatgggcagtataagaagacagccagcaatggcaagcttcgggcctacttgcctgatttccgtttcacacccttcaagcaggctgtgaaggagacctgtgcctggttcaccgacaactatgagcaggcccggaagtgaagcatgggacaagcgggtgctcagctggcaatgcccagtcagtaggctgcagtctcatcatttgcttgtcaagaactgaggacagtatccagcaacctgagccacatgctggtctctctgccagggggcttcatgcagccatccagtagggcccatgtttgtccatcctcgggggaaggccagaccaacaccttgtttgtctgcttctgccccaacctcagtgcatccatgctggtcctgctgtcccttgtctaga

SEQ ID NO: 4 - protein, 321 aa, Cricetulus griseus
MGEPQGSRRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGTRKVVSCLSTCIFPDKTTYPIDETMIHNGPPHSSNFGYSYAKRMIDVQNRAYFQQHGCTFTAVIPTNVFGPHDNFNIEDGHVLPGLIHKVHLAKSNGSALTVWGTGKPRRQFIYSLDLARLFIWVLREYNEVEPIILSVGEEDEVSIKEAAEAVVEAMDFCGEVTFDSTKSDGQYKKTASNGKLRAYLPDFRFTPFKQAVKETCAWFTDNYEQARK

SEQ ID NO: 5 - DNA, 2008 bp, Cricetulus griseus
aacagaaacttattttcctgtgtggctaactagaaccagagtacaatgtttccaattctttgagctccgagaagacagaagggagttgaaactctgaaaatgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccgaataccagaaggccctattgatcaggggacagctacaggaagagtccgtgttttagaagaacagcttgttaaggccaaagaacagattgaaaattacaagaaacaagctaggaatgatctgggaaaggatcatgaaatcttaaggaggaggattgaaaatggagctaaagagctctggttttttctacaaagtgaattgaagaaattaaagaaattagaaggaaacgaactccaaagacatgcagatgaaattcttttggatttaggacatcatgaaaggtctatcatgacagatctatactacctcagtcaaacagatggagcaggtgagtggcgggaaaaagaagccaaagatctgacagagctggtccagcggagaataacatatctgcagaatcccaaggactgcagcaaagccagaaagctggtatgtaatatcaacaaaggctgtggctatggatgtcaactccatcatgtggtttactgcttcatgattgcttatggcacccagcgaacactcatcttggaatctcagaattggcgctatgctactggaggatgggagactgtgtttagacctgtaagtgagacatgcacagacaggtctggcctctccactggacactggtcaggtgaagtgaaggacaaaaatgttcaagtggtcgagctccccattgtagacagcctccatcctcgtcctccttacttacccttggctgtaccagaagaccttgcagatcgactcctgagagtccatggtgatcctgcagtgtggtgggtatcccagtttgtcaaatacttgatccgtccacaaccttggctggaaagggaaatagaagaaaccaccaagaagcttggcttcaaacatccagttattggagtccatgtcagacgcactgacaaagtgggaacagaagcagccttccatcccattgaggaatacatggtacacgttgaagaacattttcagcttctcgaacgcagaatgaaagtggataaaaaaagagtgtatctggccactgatgacccttctttgttaaaggaggcaaagacaaagtactccaattatgaatttattagtgataactctatttcttggtcagctggactacacaaccgatacacagaaaattcacttcggggcgtgatcctggatatacactttctctcccaggctgacttccttgtgtgtactttttcatcccaggtctgtagggttgcttatgaaatcatgcaaacactgcatcctgatgcctctgcaaacttccattctttagatgacatctactattttggaggccaaaatgcccacaaccagattgcagtttatcctcaccaacctcgaactaaagaggaaatccccatggaacctggagatatcattggtgtggctggaaaccattggaatggttactctaaaggtgtcaacagaaaactaggaaaaacaggcctgtacccttcctacaaagtccgagagaagatagaaacagtcaaataccctacatatcctgaagctgaaaaatagagatggagtgtaagagattaacaacagaatttagttcagaccatctcagccaagcagaagacccagactaacatatggttcattgacagacatgctccgcaccaagagcaagtgggaaccctcagatgctgcactggtggaacgcctctttgtgaagggctgctgtgccctcaagcccatg

SEQ ID NO: 6 - DNA, 1728 bp, Mus musculus
atgcgggcatggactggttcctggcgttggattatgctcattctttttgcctgggggaccttgttattttatataggtggtcatttggttcgagataatgaccaccctgatcactccagcagagaactctccaagattcttgcaaagcttgaacgcttaaaacagcaaaatgaagacttgaggcgaatggctgagtctctccgaataccagaaggccccattgaccaggggacagctacaggaagagtccgtgttttagaagaacagcttgttaaggccaaagaacagattgaaaattacaagaaacaagctagaaatggtctggggaaggatcatgaaatcttaagaaggaggattgaaaatggagctaaagagctctggttttttctacaaagcgaactgaagaaattaaagcatttagaaggaaatgaactccaaagacatgcagatgaaattcttttggatttaggacaccatgaaaggtctatcatgacagatctatactacctcagtcaaacagatggagcaggggattggcgtgaaaaagaggccaaagatctgacagagctggtccagcggagaataacatatctccagaatcctaaggactgcagcaaagccaggaagctggtgtgtaacatcaataaaggctgtggctatggttgtcaactccatcacgtggtctactgtttcatgattgcttatggcacccagcgaacactcatcttggaatctcagaattggcgctatgctactggtggatgggagactgtgtttagacctgtaagtgagacatgtacagacagatctggcctctccactggacactggtcaggtgaagtaaatgacaaaaacattcaagtggtcgagctccccattgtagacagcctccatcctcggcctccttacttaccactggctgttccagaagaccttgcagaccgactcctaagagtccatggtgaccctgcagtgtggtgggtgtcccagtttgtcaaatacttgattcgtccacaaccttggctggaaaaggaaatagaagaagccaccaagaagcttggcttcaaacatccagttattggagtccatgtcagacgcacagacaaagtgggaacagaagcagccttccaccccatcgaggagtacatggtacacgttgaagaacattttcagcttctcgcacgcagaatgcaagtggataaaaaaagagtatatctggctactgatgatcctactttgttaaaggaggcaaagacaaagtactccaattatgaatttattagtgataactctatttcttggtcagctggactacacaatcggtacacagaaaattcacttcggggtgtgatcctggatatacactttctctcacaggctgactttctagtgtgtactttttcatcccaggtctgtcgggttgcttatgaaatcatgcaaaccctgcatcctgatgcctctgcgaacttccattctttggatgacatctactattttggaggccaaaatgcccacaatcagattgctgtttatcctcacaaacctcgaactgaagaggaaattccaatggaacctggagatatcattggtgtggctggaaaccattgggatggttattctaaaggtatcaacagaaaacttggaaaaacaggcttatatccctcctacaaagtccgagagaagatagaaacagtcaagtatcccacatatcctgaagctgaaaaatag

SEQ ID NO: 7 - protein, 575 aa, Cricetulus griseus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNDLGKDHEILRRRIENGAKELWFFLQSELKKLKKLEGNELQRHADEILLDLGHHERSIMTDLYYLSQTDGAGEWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVKDKNVQVVELPIVDSLHPRPPYLPLAVPEDLADRLLRVHGDPAVWWVSQFVKYLIRPQPWLEREIEETTKKLGFKHPVIGVHVRRTDKVGTEAAFHPIEEYMVHVEEHFQLLERRMKVDKKRVYLATDDPSLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHQPRTKEEIPMEPGDIIGVAGNHWNGYSKGVNRKLGKTGLYPSYKVREKIETVKYPTYPEAEK

SEQ ID NO: 8 - protein, 575 aa, Mus musculus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 9 - DNA, 383 bp, Cricetulus griseus
gttaactggggctcttttaaaccctgaatttttctaaatccccacctccaagagtttggtttaaactgatttttttaatgaataccttttgaagaatagagcattgtctcatcatgcaaagcttctcagggattcagctagcatgttgaagaaacataagggtgttaaattgtttgtcacaagtgctgaataaatattgacgtagtcttcagctattctatactggaagtagatgatattctcattggaaattctgttaggaagtaacccttcttgtcttcttacctgcatagaatcccaggatataaaacttgtgcttgtcgcccttgccattgtctctcactggtggcctttattgcatctcatatctgccttctctttcc

SEQ ID NO: 10 - DNA, 564 bp, Cricetulus griseus
taagaattcctgtgcccagctgtatgtgaggctctctgcaggtgtagggatgtttctgctttctttctgcacatgcttcacagctgaagtcctttgggtgtgagattgacattcagatagactaaagtgactggacttgttgggaaacatactgtatgcattattgccgttgcctccaggtgaaattaacacctcattcaccaatccctgttcatccaaactttctacccacatcactttaaatagaaattagacccaatatgactccttttttcctaagctgtttatagagattgtgctggagcagtgagcttttgtgtttgtttgtttgttttgtaattttccccatgaaaatttctctaaactcaaacctaagagggaaaaaaaaaaaacagacttatatgtgccacacttgtaaaaaaaaatcatgaaagatgtatatgatatttttaaacagtttgaatattaagatcacaatttctattttaaaaacaatcttgttttacatatcaatcacccaattcccttgccttcccatcctcccattccccccactgatccccc

SEQ ID NO: 11 - DNA, 120 bp, Cricetulus griseus
atgaatgttcattctttgggtatatgcccaagagtagaattgctaaatattgaggtagactgattcccattttcttgaggagtcgccatattgatttccaaagtgactgtacaagttaac

SEQ ID NO: 12 - DNA, 274 bp, Cricetulus griseus
aggcactaggtaaatatttttgaagaaagaatgagtatctcctatttcagaaaaacttttattgacttaaatttaggatatcagaattagaaaacagtaaaaatttataggagagtttttaatgaatgttattttaaggttccatacaaatagtaattaaaacttacacaaactatttgtagtaatgattcagtctggtataccctgatgagcattatacacttttaaattctttttgtaaatttttttattagttcaaattaggaacaagctt

SEQ ID NO: 13 - DNA, 9196 bp, Cricetulus griseus
tctagaccaggctggtctcgaactcacagagaaccacctgcctctgccacctgagtgctgggattaaaggtgtgcaccaccaccgcccggcgtaaaatcatatttttgaatattgtgataatttacattataattgtaagtaaaaattttcagcctattttgttatacatttttgcgtaaattattcttttttgaaagttttgttgtccataatagtctagggaaacataaagttataatttttgtctatgtatttgcatatatatctatttaatctcctaatgtccaggaaataaatagggtatgtaatagcttcaacatgtggtatgatagaatttttcagtgctatataagttgttacagcaaagtgttattaattcatatgtccatatttcaattttttatgaattattaaattgaatccttaagctgccagaactagaattttattttaatcaggaagccccaaatctgttcattctttctatatatgtggaaaggtaggcctcactaactgattcttcacctgttttagaacatggtccaagaatggagttatgtaaggggaattacaagtgtgagaaaactcctagaaaacaagatgagtcttgtgaccttagtttctttaaaaacacaaaattcttggaatgtgttttcatgttcctcccaggtggataggagtgagtttatttcagattatttattacaactggctgttgttacttgtttctatgtctttatagaaaaacatattttttttgccacatgcagcttgtccttatgattttatacttgtgtgactcttaactctcagagtataaattgtctgatgctatgaataaagttggctattgtatgagacttcagcccacttcaattattggcttcattctctcagatcccaccacctccagagtggtaaacaacttgaaccattaaacagactttagtctttatttgaatgatagatggggatatcagatttataggcacagggttttgagaaagggagaaggtaaacagtagagtttaacaacaacaaaaagtatactttgtaaacgtaaaactatttattaaagtagtagacaagacattaaatattccttgggattagtgctttttgaattttgctttcaaataatagtcagtgagtatacccctcccccattctatattttagcagaaatcagaataaatggtgtttctggtacattcttttgtagagaatttattttctttgggtttttgtgcatttaaagtcaataaaaattaaggttcagtaatagaaaaaaaactctgatttttggaatcccctttcttcagcttttctatttaatctcttaatgataatttaatttgtggccatgtggtcaaagtatatagccttgtatatgtaaatgttttaaccaacctgcctttacagtaactatataattttattctataatatatgacttttcttccatagctttagagttgcccagtcactttaagttacattttcatatatgttctttgtgggaggagataattttatttctaagagaatcctaagcatactgattgagaaatggcaaacaaaacacataattaaagctgataaagaacgaacatttggagtttaaaatacatagccaccctaagggtttaactgttgttagccttcttttggaatttttattagttcatatagaaaaatggattttatcgtgacatttccatatatgtatataatatatttacatcatatccacctgtaattattagtgtttttaaatatatttgaaaaaataatggtctggtttgatccatttgaaccttttgatgtttggtgtggttgccaattggttgatggttatgataacctttgcttctctaaggttcaagtcagtttgagaatatgtcctctaaaaatgacaggttgcaagttaagtagtgagatgacagcgagatggagtgatgagaatttgtagaaatgaattcacttatactgagaacttgttttgcttttagataatgaacatattagcctgaagtacatagccgaattgattaattattcaaagatataatcttttaatccctataaaagaggtattacacaacaattcaagaaagatagaattagacttccagtattggagtgaaccatttgttatcaggtagaaccctaacgtgtgtggttgacttaaagtgtttactttttacctgatactgggtagctaattgtctttcagcctcctggccaaagataccatgaaagtcaacttacgttgtattctatatctcaaacaactcagggtgtttcttactctttccacagcatgtagagcccaggaagcacaggacaagaaagctgcctccttgtatcaccaggaagatctttttgtaagagtcatcacagtataccagagagactaattttgtctgaagcatcatgtgttgaaacaacagaaacttattttcctgtgtggctaactagaaccagagtacaatgtttccaattctttgagctccgagaagacagaagggagttgaaactctgaaaatgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccggtaggtttgaaatactcaaggatttgatgaaatactgtgcttgacctttaggtatagggtctcagtctgctgttgaaaaatataatttctacaaaccgtctttgtaaaattttaagtattgtagcagactttttaaaagtcagtgatacatctatatagtcaatataggtttacatagttgcaatcttattttgcatatgaatcagtatatagaagcagtggcatttatatgcttatgttgcatttacaattatgtttagacgaacacaaactttatgtgatttggattagtgctcattaaatttttttattctatggactacaacagagacataaattttgaaaggcttagttactcttaaattcttatgatgaaaagcaaaaattcattgttaaatagaacagtgcatccggaatgtgggtaattattgccatatttctagtctactaaaaattgtggcataactgttcaaagtcatcagttgtttggaaagccaaagtctgatttaaatggaaaacataaacaatgatatctatttctagatacctttaacttgcagttactgagtttacaagttgtctgacaactttggattctcttacttcatatctaagaatgatcatgtgtacagtgcttactgtcactttaaaaaactgcagggctagacatgcagatatgaagactttgacattagatgtggtaattggcactaccagcaagtggtattaagatacagctgaatatattactttttgaggaacataattcatgaatggaaagtggagcattagagaggatgccttctggctctcccacaccactgtttgcatccattgcatttcacactgcttttagaactcagatgtttcatatggtatattgtgtaactcaccatcagttttatctttaaatgtctatggatgataatgttgtatgttaacacttttacaaaaacaaatgaagccatatcctcggtgtgagttgtgatggtggtaattgtcacaataggattattcagcaaggaactaagtcagggacaagaagtgggcgatactttgttggattaaatcattttactggaagttcatcagggagggttatgaaagttgtggtctttgaactgaaattatatgtgattcattattcttgatttaggccttgctaatagtaactatcatttattgggaatttgtcatatgtgccaatttgtcatgggccagacagcgtgttttactgaatttctagatatctttatgagattctagtactgttttcagccattttacagatgaagaatcttaaaaaatgttaaataatttagtttgcccaagattatacgttaacaaatggtagaaccttctttgaattctggcagtatggctacacagtccgaactcttatcttcctaagctgaaaacagaaaaagcaatgacccagaaaattttatttaaaagtctcaggagagacttcccatcctgagaagatctcttttcccttttataatttaggctcctgaataatcactgaattttctccatgttccatctatagtactgttatttctgttttccttttttcttaccacaaagtatcttgtttttgctgtatgaaagaaaatgtgttattgtaatgtgaaattctctgtccctgcagggtcccacatccgcctcaatcccaaataaacacacagaggctgtattaattatgaaactgttggtcagttggctagggcttcttattggctagctctgtcttaattattaaaccataactactattgtaagtatttccatgtggtcttatcttaccaaggaaagggtccagggacctcttactcctctggcgtgttggcagtgaagaggagagagcgatttcctatttgtctctgcttattttctgattctgctcagctatgtcacttcctgcctggccaatcagccaatcagtgttttattcattagccaataaaagaaacatttacacagaaggacttcccccatcatgttatttgtatgagttcttcagaaaatcatagtatcttttaatactaatttttataaaaaattaattgtattgaaaattatgtgtatatgtgtctgtgtgtcgatttgtgctcataagtagcatggagtgcagaagagggaatcagatctttttttaagggacaaagagtttattcagattacattttaaggtgataatgtatgattgcaaggttatcaacatggcagaaatgtgaagaagctggtcacattacatccagagtcaagagtagagagcaatgaattgatgcatgcattcctgtgctcagctcacttttcctggagctgagctgattgtaagccatctgatgtctttgctgggaactaactcaaaggcaagttcaaaacctgttcttaagtataagccatctctccagtccctcatatggtctcttaagacactttctttatattcttgtacatagaaattgaattcctaacaactgcattcaaattacaaaatagtttttaaaagctgatataataaatgtaaatacaatctagaacatttttataaataagcatattaactcagtaaaaataaatgcatggttattttccttcattagggaagtatgtctccccaggctgttctctagattctactagtaatgctgtttgtacaccatccacaggggttttattttaaagctaagacatgaatgatggacatgcttgttagcatttagacttttttccttactataattgagctagtatttttgtgctcagtttgatatctgttaattcagataaatgtaatagtaggtaatttctttgtgataaaggcatataaattgaagttggaaaacaaaagcctgaaatgacagtttttaagattcagaacaataattttcaaaagcagttacccaactttccaaatacaatctgcagttttcttgatatgtgataaatttagacaaagaaatagcacattttaaaatagctatttactcttgatttttttttcaaatttaggctagttcactagttgtgtgtaaggttatggctgcaaacatctttgactcttggttagggaatccaggatgatttacgtgtttggccaaaatcttgttccattctgggtttcttctctatctaggtagctagcacaagttaaaggtgtggtagtattggaaggctctcaggtatatatttctatattctgtatttttttcctctgtcatatatttgctttctgttttattgatttctactgttagtttgatacttactttcttacactttctttgggatttattttgctgttctaagatttcttagcaagttcatatcactgattttaacagttgcttcttttgtaatatagactgaatgccccttatttgaaatgcttgggatcagaaactcagatttgaacttttcttttttaatatttccatcaagtttaccagctgaatgtcctgatccaagaatatgaaatctgaaatgctttgaaatctgaaacttttagagtgataaagcttccctttaaattaatttgtgttctatattttttgacaatgtcaacctttcattgttatccaatgagtgaacatattttcaatttttttgtttgatctgttatattttgatctgaccatatttataaaattttatttaatttgaatgttgtgctgttacttatctttattattatttttgcttattttctagccaaatgaaattatattctgtattattttagtttgaattttactttgtggcttagtaactgccttttgttggtgaatgcttaagaaaaacgtgtggtctactgatattggttctaatcttatatagcatgttgtttgttaggtagttgattatgctggtcagattgtcttgagtttatgcaaatgtaaaatatttagatgcttgttttgttgtctaagaacaaagtatgcttgctgtctcctatcggttctggtttttccattcatctcttcaagctgttttgtgtgttgaatactaactccgtactatcttgttttctgtgaattaaccccttttcaaaggtttcttttctttttttttttaagggacaacaagtttattcagattacattttaagctgataatgtatgattgcaaggttatcaacatggcagaaatgtgaagaagctaggcacattacatccacatggagtcaagagcagagagcagtgaattaatgcatgcattcctgtggtcagctcacttttcctattcttagatagtctaggatcataaacctggggaatagtgctaccacaatgggcatatccacttacttcagttcatgcaatcaaccaaggcacatccacaggaaaaactgatttagacaacctctcattgagactcttcccagatgattagactgtgtcaagttgacaattaaaactatcacacctgaagccatcactagtaaatataatgaaaatgttgattatcaccataattcatctgtatccctttgttattgtagattttgtgaagttcctattcaagtccctgttccttccttaaaaacctgttttttagttaaataggttttttagtgttcctgtctgtaaatacttttttaaagttagatattattttcaagtatgttctcccagtctttggcttgtattttcatcccttcaatacatatatttttgtaatttattttttttatttaaattagaaacaaagctgcttttacatgtcagtctcagttccctctccctcccctcctcccctgctccccacctaagccccaattccaactcctttcttctccccaggaagggtgaggccctccatgggggaaatcttcaatgtctgtcatatcatttggagcagggcctagaccctccccagtgtgtctaggctgagagagtatccctctatgtggagagggctcccaaagttcatttgtgtactaggggtaaatactgatccactatcagtggccccatagattgtccggacctccaaactgacttcctccttcagggagtctggaacagttctatgctggtttcccagatatcagtctggggtccatgagcaaccccttgttcaggtcagttgtttctgtaggtttccccagcccggtcttgacccctttgctcatcacttctccctctctgcaactggattccagagttcagctcagtgtttagctgtgggtgtctgcatctgcttccatcagctactggatgagggctctaggatggcatataaggtagtcatcagtctcattatcagagaagggcttttaaggtagcctcttgattattgcttagattgttagttggggtcaaccttgtaggtctctggacagtgacagaattctctttaaacctataatggctccctctgtggtggtatcccttttcttgctctcatccgttcctcccctgactagatcttcctgctccctcatgtcctcctctcccctccccttctccccttctctttcttctaactccctctcccctccacccacgatccccattagcttatgagatcttgtccttattttagcaaaacctttttggctataaaattaattaatttaatatgcttatatcaggtttattttggctagtatttgtatgtgtttggttagtgtttttaaccttaattgacatgtatccttatatttagacacagatttaaatatttgaagttttttttttttttttttttaaagatttatttattttttatgtcttctgcctgcatgccagaagagggcaccagatctcattcaaggtggttgtgagccaccatgtggttgctgggaattgaactcaggacctctggaagaacagtcagtgctcttaaccgctgagccatctctccagcccctgaagtgtttcttttaaagaggatagcagtgcatcatttttccctttgaccaatgactcctaccttactgaattgttttagccatttatatgtaatgctgttaccaggtttacattttcttttatcttgctaaatttcttccctgtttgtctcatctcttatttttgtctgttggattatataggcttttatttttctgtttttacagtaagttatatcaaattaaaattattttatggaatgggtgtgttgactacatgtatgtctgtgcaccatgtgctgacctggtcttggccagaagaaggtgtcatattctctgaaactggtattgtggatgttacgaactgccatagggtgctaggaatcaaaccccagctcctctggaaaagcagccactgctctgagccactgagtcctctcttcaagcaggtgatgccaacttttaatggttaccagtggataagagtgcttgtatctctagcacccatgaaaatttatgcattgctatatgggcttgtcacttcagcattgtgtgacagagacaggaggatcccaagagctc

SEQ ID NO: 14 - protein, 5 aa, Mus musculus
NYGMS

SEQ ID NO: 15 - protein, 17 aa, Mus musculus
TISSASTYSYYPDSVKG

SEQ ID NO: 16 - protein, 10 aa, Mus musculus
HSDGNFAFGY

SEQ ID NO: 17 - protein, 16 aa, Mus musculus
RSSRNIVHINGDTYLE

SEQ ID NO: 18 - protein, 7 aa, Mus musculus
KVSNRFS

SEQ ID NO: 19 - protein, 9 aa, Mus musculus
FQGSLLFWT

SEQ ID NO: 20 - DNA, 414 bp, Mus musculus
  CDS (1)..(414)
atgaacctcgggctcagtttgattttccttgccctcattttaaaaggtgtccagtgtgaggtgcagctggtggagtctgggggagacttaatgaagcctggagggtccctgaaaatctcctgtgcagcctctggattcattttcagtaattatggcatgtcttgggttcgccagactccagacatgaggctggaatgggtcgcaaccattagtagtgctagtacttattcctattatccagacagtgtgaagggacgattcaccatatccagggacaacgccgagaactccctatatctgcaaatgaatagtctgaggtctgaggacacaggcatatattactgtggaagacatagcgatggaaacttcgcgtttggttattggggccgagggactctggtcactgtctctgca

SEQ ID NO: 21 - protein, 119 aa, Mus musculus
EVQLVESGGDLMKPGGSLKISCAASGFIFSNYGMSWVRQTPDMRLEWVATISSASTYSYYPDSVKGRFTISRDNAENSLYLQMNSLRSEDTGIYYCGRHSDGNFAFGYWGRGTLVTVSA

SEQ ID NO: 22 - DNA, 396 bp, Mus musculus
  CDS (1)..(396)
atgaagttgcctgttaggctgttggtgctgatgttctggattcctgcttccagcagtgatgttttgatgacccaaactccactctccctgcctgtcagtcttggagatcaagcctccatctcttgcagatctagtcggaacattgttcatattaatggtgacacatatttagaatggtacctgcagagaccgggccagtctccaaagctcctaatctacaaagtttccaaccgattttctggggtcccagacaggttcagtggcagtggatcagggacagatttcacactcaagatcagcagagtggaggctgaggatctgggagtttattactgctttcaaggttcacttcttccgtggacgttcggtggaggcaccaggctggaaatcagacgg

SEQ ID NO: 23 - protein, 113 aa, Mus musculus
DVLMTQTPLSLPVSLGDQASISCRSSRNIVHINGDTYLEWYLQRPGQSPKLLIYKVSNRFSGVPDRFSGSGSGTDFTLKISRVEAEDLGVYYCFQGSLLPWTFGGGTRLEIRR

SEQ ID NO: 24 - protein, 119 aa, Artificial Sequence
  Synthetic peptide
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCARHSDGNFAFGYWGQGTLVTVSS

SEQ ID NO: 25 - protein, 119 aa, Artificial Sequence
  Synthetic peptide
EVQLVESGGGLVQPGGSLRLSCAASGFTFSNYGMSWVRQAPGKGLEWVSTISSASTYSYYPDSVKGRFTISRDNSKNTLYLQMNSLRAEDTAVYYCARHSDGNFAFGYWGQGTLVTVSS

SEQ ID NO: 26 - protein, 112 aa, Artificial Sequence
  Synthetic peptide
DIVMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 27 - protein, 119 aa, Artificial Sequence
  Synthetic peptide
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 28 - protein, 119 aa, Artificial Sequence
  Synthetic peptide
EVQLVESGGDLVQPGRSLRLSCAASGFIFSNYGMSWVRQAPDKRLEWVATISSASTYSYYPDSVKGRFTISRDNAKNSLYLQMNSLRVEDTALYYCARHSDGNFAFGYWGQGTLVTVSS

SEQ ID NO: 29 - protein, 119 aa, Artificial Sequence
  Synthetic peptide
EVQLVESGGDLVQPGRSLRLSCAASGFIFSNYGMSWVRQAPDKRLEWVATISSASTYSYYPDSVKGRFTISRDNAKNSLYLQMNSLRVEDTALYYCGRHSDGNFAFGYWGQGTLVTVSS

SEQ ID NO: 30 - protein, 119 aa, Artificial Sequence
  Synthetic peptide
EVQLVESGGGLVQPGGSLRLSCAASGFIFSNYGMSWVRQAPGKGLEWVSTISSASTYSYYPDSVKGRFTISRDNSKNTLYLQMNSLRAEDTAVYYCARHSDGNFAFGYWGQGTLVTVSS

SEQ ID NO: 31 - protein, 119 aa, Artificial Sequence
  Synthetic peptide
EVQLVESGGGLVQPGGSLRLSCAASGFTFSNYGMSWVRQAPGKGLEWVSTISSASTYSYYPDSVKGRFTISRDNSKNTLYLQMNSLRAEDTAVYYCGRHSDGNFAFGYWGQGTLVTVSS

SEQ ID NO: 32 - protein, 119 aa, Artificial Sequence
  Synthetic peptide
EVQLVESGGGLVQPGGSLRLSCAASGFIFSNYGMSWVRQAPGKGLEWVSTISSASTYSYYPDSVKGRFTISRDNSKNTLYLQMNSLRAEDTAVYYCGRHSDGNFAFGYWGQGTLVTVSS

SEQ ID NO: 33 - protein, 112 aa, Artificial Sequence
  Synthetic peptide
DVVMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 34 - protein, 112 aa, Artificial Sequence
  Synthetic peptide
DILMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 35 - protein, 112 aa, Artificial Sequence
  Synthetic peptide
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 36 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 37 - protein, 28 aa, Homo sapiens
NPTDIADTTLDESIYSNYYLYESIPKPC

SEQ ID NO: 38 - protein, 18 aa, Homo sapiens
DESIYSNYYLYESIPKPC

SEQ ID NO: 39 - DNA, 28 bp, Artificial Sequence
  Description of Artificial Sequence Synthetic DNA
gagacttcagcccacttcaattattggc

SEQ ID NO: 40 - DNA, 25 bp, Artificial Sequence
  Description of Artificial Sequence Synthetic DNA
cttgtgtgactcttaactctcagag

SEQ ID NO: 41 - DNA, 25 bp, Artificial Sequence
  Description of Artificial Sequence Synthetic DNA
gaggccacttgtgtagcgccaagtg

SEQ ID NO: 42 - DNA, 23 bp, Artificial Sequence
  Description of Artificial Sequence Synthetic DNA
ccctcgagataacttcgtatagc

SEQ ID NO: 43 - DNA, 18 bp, Artificial Sequence
  Description of Artificial Sequence Synthetic DNA
ggtaggcctcactaactg

SEQ ID NO: 44 - DNA, 25 bp, Artificial Sequence
  Description of Artificial Sequence Synthetic DNA
catagaaacaagtaacaacagccag

SEQ ID NO: 45 - DNA, 21 bp, Artificial Sequence
  Description of Artificial Sequence Synthetic DNA
gtgagtccatggctgtcactg

SEQ ID NO: 46 - DNA, 20 bp, Artificial Sequence
  Description of Artificial Sequence Synthetic DNA
cctgacttggctattctcag